(19) United States
(12) Patent Application Publication
(10) Pub. No.: US 2026/0028637 A1
(43) Pub. Date: Jan. 29, 2026

(54) PROMOTERS FOR REGULATION OF GENE EXPRESSION IN PLANTS

(71) Applicant: Syngenta Crop Protection AG, Basel (CH)

(72) Inventors: Kasimalai Azhakanandam, RTP, NC (US); Ailing Zhou, RTP, NC (US); Jared Conville, RTP, NC (US); Joseph Dallas Clarke, V, RTP, NC (US)

(73) Assignee: Syngenta Crop Protection AG, Basel (CH)

(21) Appl. No.: 18/946,276

(22) Filed: Nov. 13, 2024

Related U.S. Application Data
(62) Division of application No. 17/640,963, filed on Mar. 7, 2022, now Pat. No. 12,173,297.
(60) Provisional application No. 62/896,735, filed on Sep. 6, 2019.

(51) Int. Cl. C12N 15/82

(52) U.S. Cl. CPC C12N 15/8223; C12N 15/8205; C12N 15/8286

(57) ABSTRACT

The present invention is directed to promoters that have particular utility in driving root-specific expression of heterologous genes that impart increased agronomic, horticultural and/or pesticidal characteristics to a given transgenic plant. The present invention is also drawn to DNA molecules comprising the promoters of the invention and transformed plant tissues containing DNA molecules comprising a promoter of the invention operably linked to a heterologous gene or genes, and seeds thereof.

### RELATED APPLICATION INFORMATION

This application is a divisional of U.S. application Ser. No. 17/640,963, filed Mar. 7, 2022, which is a 371 of International Application No. PCT/US2020/47899, filed Aug. 26, 2020, which claims priority to U.S. Provisional Application No. 62/896,735, filed Sep. 6, 2019, the contents of each of which are incorporated herein by reference herein.

### SEQUENCE LISTING

A Sequence Listing in xml format, submitted under 37 C.F.R. § 1.821, entitled “81937-US-DIV sequences_corrected.xml”, 216 kilobytes in size, generated on Sep. 12, 2025 and filed via EFS-Web is provided in lieu of a paper copy. This Sequence Listing is hereby incorporated by reference into the specification for its disclosures.

### FIELD OF THE INVENTION

The present invention relates generally to the field of plant molecular biology and the regulation of gene expression in plants. Also disclosed are transgenic eukaryotes, including transgenic plant cells, plants, and seeds, whose genome includes molecular constructs for controlling heterologous gene expression.

### BACKGROUND

Transgenic crops consist of increasingly complex genetic modifications including multiple transgenes that confer different traits, also called “gene stacks” or “trait stacks.” For example, many transgenic corn products currently on the market contain within the same plant multiple genes encoding insecticidal proteins for controlling a broad spectrum of insect pests, multiple genes encoding proteins that confer on the plant tolerance to a wide spectrum of chemical herbicides and multiple genes encoding proteins that are used as selectable markers during the plant transformation process. Many of the transgenic proteins used to control insect pests, for example the crystal endotoxins from Bacillus thuringiensis (called Cry proteins) are active against lepidopteran or coleopteran insect pests. Examples of lepidopteran-active Cry proteins include Cry1A, Cry1B, Cry1C, Cry1D, Cry1E, Cry1F and Cry9. Examples of coleopteran-active Cry proteins include, Cry3A, Cry3B, Cry3C, Cry8, the binary Cry23-Cry37 and the binary Cry34-Cry35. Most individual Cry proteins are biologically active against a narrow spectrum of insect species within a given insect order. Even with this narrow spectrum of activity, certain Cry proteins may have low to moderate activity against certain non-pest species in the same order of insects as the target pest insects. For example, Hellmich et al. (2001) Proc. Natl. Aca. Sci. 98:11925-11930, found that certain purified Cry proteins that are active against a lepidopteran pest, e.g. European corn borer (Ostrinia nubilalis), also have some activity against the first instar of the non-pest lepidopteran insect, monarch butterfly (Danaus plexippus). However, later larval instars D. plexippus were far less susceptible.

Currently, expression of most transgenes encoding insecticidal proteins in commercial transgenic crops is driven by constitutive promoters, i.e. promoters that are functional throughout the plant in all or a majority of tissue types, including pollen, throughout the entire growth cycle of the plant. Since plant pollen may be a food source for some non-pest insect species or it is hypothesized that plant pollen may be carried by wind to deposit on non-pest insect host plants, there is some concern within regulatory agencies that regulate transgenic crop commercialization that high levels of expression of certain insecticidal proteins, e.g. certain Cry proteins, in pollen may have adverse effects on localized populations of non-pest insects. In addition, it has been observed that expression of certain insecticidal proteins in pollen has adverse effects on the transgenic plant's male fertility. For example, high levels of a Vip3 insecticidal protein expressed in corn pollen may cause a decrease in male fertility or complete sterility in certain inbred genetic backgrounds (U.S. Pat. No. 10,214,784; herein incorporated by reference). Therefore, it would be beneficial to modulate the expression of insecticidal proteins in transgenic plants, for example, to have high levels of expression in vegetative tissues, e.g. leaf tissue, where a majority of pest insects initially feed, but have reduced expression in pollen, a plant tissue that some non-pest insects may feed upon.

It is, therefore, desirable to provide plants, particularly corn plants that exclude expression of a transgene in the tissues of the reproductive structures of the plant such as pollen and/or the tassel. This could be achieved within the scope of the present invention by providing a regulatory nucleotide sequence, at least part of which has a transcription initiation function directing expression of an operably linked polynucleotide encoding a protein of interest to basically all plant tissues, but essentially excluding expression in the tissues of the male reproductive structures of the plant, particularly in pollen and/or tassel tissues so that little or no expression product is present in those tissues. The regulatory nucleotide sequence can then be used to develop expression systems that enable effective accumulation of the protein of interest such as, for example, an insecticidal protein, in tissues that target pests normally feed on, and eliminate or reduce accumulation of the insecticidal protein in non-target tissues or organs and/or in those tissues that may be compromised by the protein of interest.

### SUMMARY

The present invention provides compositions and methods for selectively directing transgene expression in transgenic plant tissues. In particular, novel promoter polynucleotides capable of initiating and/or modulating transcription of a DNA polynucleotide to which they are operably linked are provided. The promoters of the invention are characterized by their ability to selectively modulate expression of any operably linked DNA polynucleotide, such as a polynucleotide encoding an insecticidal protein, preferentially in multiple plant tissues such as leaves, stems, roots, and the like, but not in male reproductive tissues such as pollen.

In some aspects, a promoter polynucleotide of the invention comprises any of SEQ ID NOS: 1-11 or their complements, or a fragment, region, cis element, or polynucleotides related to any of SEQ ID NOS: 1-11, that function as promoters in plants. The promoters of the invention direct functional transcription in any plant tissue other than a male reproductive tissue, e.g. pollen, in which transcription is eliminated or significantly reduced compared to other non-male reproductive tissues of the plant.

The invention is further drawn to chimeric constructs, expression cassettes and vectors comprising a promoter of the invention operably linked to a heterologous DNA polynucleotide which encodes a protein which is desired to be expressed in plant tissues other than pollen. For example, a recombinant DNA molecule of the invention may comprise, in a 5′ to 3′ direction, a promoter of the invention operably linked to a DNA sequence which encodes an insecticidal protein that is active against insect pests that feed on plant tissues and a terminator sequence operably linked to the 3′ end of the gene of interest. In some aspects, the terminator sequence of the invention comprises any of SEQ ID NOS:12-20 or their complements, or a fragment, region, or nucleotide sequences related to any of SEQ ID NOS:12-20. In other aspects, a vector of the invention is a binary vector useful for plant transformation. In other aspects, the binary vector comprises any of SEQ ID NOS:21-28 or sequences related to any of SEQ ID NOS:21-28.

In other aspects, the regulatory sequences of the invention are operably linked to a nucleotide sequence that encodes an insecticidal protein. Therefore, upon insertion of the expression cassette comprising a promoter of the invention operably linked to the insecticidal protein coding sequence into a genome of a plant, the resulting transgenic plant will be protected from attack by pest insects which feed on non-pollen tissues, such as leaves and stems, but will not impact beneficial insects that feed on pollen.

The invention is still further drawn to transgenic plants, such as transgenic maize plants, comprising a promoter, a chimeric construct, an expression cassette or a vector of the invention.

The invention also provides methods for specifically expressing a heterologous coding sequence in transgenic plant tissues excluding certain male reproductive tissues, for example pollen, by incorporating into the plant's genome a recombinant DNA molecule containing a promoter of the invention operably linked to a DNA polynucleotide that encodes a protein of interest, such as an insecticidal protein.

The invention further provides a method of mitigating male sterility in a transgenic plant, for example an inbred corn plant, by incorporating into the plant's genome a recombinant DNA molecule containing a promoter of the invention operably linked to a DNA polynucleotide that encodes a protein that causes male sterility when expressed in a male reproductive tissue such as pollen. In some aspects, the protein is a Vip3 insecticidal protein and the transgenic plant is an inbred corn plant.

The foregoing and other aspects of the invention will be apparent from the following detailed description, accompanying drawings and sequence listings.

### BRIEF DESCRIPTION OF THE SEQUENCES IN THE SEQUENCE LISTING

SEQ ID NO:1 is a nucleotide sequence of a PMP370-3 promoter.
SEQ ID NO:2 is a nucleotide sequence of a PMP393-1 promoter.
SEQ ID NO:3 is a nucleotide sequence of a PMP393-2 promoter.
SEQ ID NO:4 is a nucleotide sequence of a PMP393-3 promoter.
SEQ ID NO:5 is a nucleotide sequence of a PMP393-4 promoter.
SEQ ID NO:6 is a nucleotide sequence of a PMP855-1 promoter.
SEQ ID NO:7 is a nucleotide sequence of a PMP747-1 promoter.
SEQ ID NO:8 is a nucleotide sequence of a PMP004-1 promoter.
SEQ ID NO:9 is a nucleotide sequence of a PMP335-1 promoter.
SEQ ID NO:10 is a nucleotide sequence of a PMP722-1 promoter.
SEQ ID NO:11 is a nucleotide sequence of a PMP948-2 promoter.
SEQ ID NO:12 is a nucleotide sequence of a t370-2 terminator.
SEQ ID NO:13 is a nucleotide sequence of a t393-1 terminator.
SEQ ID NO:14 is a nucleotide sequence of a t393-2 terminator.
SEQ ID NO:15 is a nucleotide sequence of a t855-1 terminator.
SEQ ID NO:16 is a nucleotide sequence of a t747-1 terminator.
SEQ ID NO:17 is a nucleotide sequence of a t004-1 terminator.
SEQ ID NO:18 is a nucleotide sequence of a t335-1 terminator.
SEQ ID NO:19 is a nucleotide sequence of a t722-1 terminator.
SEQ ID NO:20 is a nucleotide sequence of a t948-2 terminator.
SEQ ID NO:21 is a nucleotide sequence of pSYN18499 binary vector.
SEQ ID NO:22 is a nucleotide sequence of pSYN18500 binary vector.
SEQ ID NO:23 is a nucleotide sequence of pSYN18501 binary vector.
SEQ ID NO:24 is a nucleotide sequence of pSYN18498 binary vector.
SEQ ID NO:25 is a nucleotide sequence of pSYN18617 binary vector.
SEQ ID NO:26 is a nucleotide sequence of pSYN18618 binary vector.
SEQ ID NO:27 is a nucleotide sequence of pSYN18619 binary vector.
SEQ ID NO:28 is a nucleotide sequence of pSYN18705 binary vector.
SEQ ID NO:29 is a nucleotide sequence of a PMP393-4:Cry1Ai expression cassette.

### DETAILED DESCRIPTION

This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

All publications, patent applications, patents and other references cited herein are incorporated by reference in their entireties for the teachings relevant to the sentence and/or paragraph in which the reference is presented.

Nucleotide sequences provided herein are presented in the 5′ to 3′ direction, from left to right and are presented using the standard code for representing nucleotide bases as set forth in 37 CFR §§ 1.821-1.825 and the World Intellectual Property Organization (WIPO) Standard ST.25, for example: adenine (A), cytosine (C), thymine (T), and guanine (G).

Amino acids are likewise indicated using the WIPO Standard ST.25, for example: alanine (Ala; A), arginine (Arg; R), asparagine (Asn; N), aspartic acid (Asp; D), cysteine (Cys; C), glutamine (Gln; Q), glutamic acid (Glu; E), glycine (Gly; G), histidine (His; H), isoleucine (Ile; 1), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

For clarity, certain terms used in the specification are defined and presented as follows:

As used herein and in the appended claims, the singular forms “a,” “an,” and “the” include plural reference unless the context clearly dictates otherwise. Thus, for example, reference to “a plant” is a reference to one or more plants and includes equivalents thereof known to those skilled in the art, and so forth.

As used herein, the term “and/or” refers to and encompasses any and all possible combinations of one or more of the associated listed items, as well as the lack of combinations when interpreted in the alternative, “or.”

The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent, preferably 10 percent up or down (higher or lower). With regard to a temperature the term “about” means ±1° C., preferably ±0.5° C. Where the term “about” is used in the context of this invention (e.g., in combinations with temperature or molecular weight values) the exact value (i.e., without “about”) is preferred.

As used herein, the term “amplified” means the construction of multiple copies of a nucleic acid molecule or multiple copies complementary to the nucleic acid molecule using at least one of the nucleic acid molecules as a template. Amplification systems include the polymerase chain reaction (PCR) system, ligase chain reaction (LCR) system, nucleic acid sequence based amplification (NASBA, Cangene, Mississauga, Ontario), Q-Beta Replicase systems, transcription-based amplification system (TAS), and strand displacement amplification (SDA). See, e.g., Diagnostic Molecular Microbiology: Principles and Applications, PERSING et al., Ed., American Society for Microbiology, Washington, D.C. (1993). The product of amplification is termed an “amplicon.”

“Activity” of an insecticidal protein is meant that the insecticidal protein functions as an orally active insect control agent, has a toxic effect, and/or is able to disrupt or deter insect feeding, which may or may not cause death of the insect. When an insecticidal protein is delivered to the insect, the result is typically death of the insect, or the insect does not feed upon the source that makes the insecticidal protein available to the insect. “Pesticidal” is defined as a toxic biological activity capable of controlling a pest, such as an insect, nematode, fungus, bacteria, or virus, preferably by killing or destroying them. “Insecticidal” is defined as a toxic biological activity capable of controlling insects, preferably by killing them. A “pesticidal agent” is an agent that has pesticidal activity. An “insecticidal agent” is an agent that has insecticidal activity.

As used herein, the terms “backcross” and “backcrossing” refer to the process whereby a progeny plant is crossed back to one of its parents for one or more generations (e.g., 2 or more, 3 or more, 4 or more, 5 or more, 6 or more, or 7 or more times, etc.). In a backcrossing scheme, the “donor” parent refers to the parental plant with the desired gene or DNA construct or locus to be introgressed. The “recipient” parent (used one or more times) or “recurrent” parent (used two or more times) refers to the parental plant into which the gene or DNA construct or locus is being introgressed. For example, see Ragot et al. Marker-assisted Backcrossing: A Practical Example, in TECHNIQUES ET UTILISATIONS DES MARQUEURS MOLECULAIRES LES COLLOQUES, Vol. 72, pp. 45-56 (1995); and Openshaw et al., Marker-assisted Selection in Backcross Breeding, in PROCEEDINGS OF THE SYMPOSIUM “ANALYSIS OF MOLECULAR MARKER DATA,” pp. 41-43 (1994). The initial cross gives rise to the F1 generation. The term “BC1” refers to the second use of the recurrent parent, “BC2” refers to the third use of the recurrent parent, and so on. In embodiments, at least one or more generations of progeny are identified and/or selected for the presence of the desired gene or locus (e.g., in a nucleic acid sample from the progeny plant or plant part). In embodiments, two or more generations (or even all generations) of progeny are identified and/or selected for the presence of the desired gene or DNA construct or locus.

The term “chimeric construct” or “chimeric gene” or “chimeric polynucleotide” or “chimeric nucleic acid” or “chimeric protein” (or similar terms) as used herein refers to a construct or nucleic acid molecule or protein comprising two or more polynucleotides or amino acid motifs or domains, respectively, of different origin assembled into a single nucleic acid molecule or protein. The term “chimeric construct”, “chimeric gene”, “chimeric polynucleotide” or “chimeric nucleic acid” refers to any construct or molecule that contains, without limitation, (1) polynucleotides (e.g., DNA), including regulatory and coding polynucleotides that are not found together in nature (i.e., at least one of the polynucleotides in the construct is heterologous with respect to at least one of its other polynucleotides), or (2) polynucleotides encoding parts of proteins not naturally adjoined, or (3) parts of promoters that are not naturally adjoined. Further, a chimeric construct, chimeric gene, chimeric polynucleotide or chimeric nucleic acid may comprise regulatory polynucleotides and coding polynucleotides that are derived from different sources, or comprise regulatory polynucleotides and coding polynucleotides derived from the same source, but arranged in a manner different from that found in nature. In some embodiments of the invention, the chimeric construct, chimeric gene, chimeric polynucleotide or chimeric nucleic acid comprises an expression cassette comprising a polynucleotide of the invention under the control of regulatory polynucleotides, particularly under the control of regulatory polynucleotides functional in plants or bacteria.

A “coding sequence” is a nucleic acid sequence that is transcribed into RNA such as mRNA, rRNA, tRNA, snRNA, sense RNA or antisense RNA. In some aspects, the RNA is then translated in an organism, such as a corn plant, to produce a protein, e.g. an insecticidal protein of the invention. In other aspects, the RNA is not translated to produce a protein but functions as an RNA molecule to modulate expression of a recombinant insecticidal protein of the invention.

As used herein, a “codon optimized” sequence means a nucleotide sequence wherein the codons are chosen to reflect the particular codon bias that a host cell or organism may have. This is typically done in such a way so as to preserve the amino acid sequence of the polypeptide encoded by the nucleotide sequence to be optimized. In certain embodiments, a DNA sequence of a recombinant DNA construct of the invention includes codons optimized for a cell (e.g., an animal, plant, or fungal cell) in which the construct is to be expressed. For example, a construct to be expressed in a plant cell can have all or parts of its sequence (e.g., the first gene suppression element or the gene expression element) codon optimized for expression in a plant. See, for example, U.S. Pat. No. 6,121,014, incorporated herein by reference.

The terms “comprises” or “comprising,” when used in this specification, specify the presence of stated features, integers, steps, operations, elements, or components, but do not preclude the presence or addition of one or more other features, integers, steps, operations, elements, components, or groups thereof.

As used herein, the transitional phrase “consisting essentially of” (and grammatical variants) means that the scope of a claim is to be interpreted to encompass the specified materials or steps recited in the claim” and those that do not materially alter the basic and novel characteristic(s)” of the claimed invention. Thus, the term “consisting essentially of” when used in a claim of this invention is not intended to be interpreted to be equivalent to “comprising.”

To “control” insects means to inhibit, through a toxic effect, the ability of insect pests to survive, grow, feed, or reproduce, or to limit insect-related damage or loss in crop plants or to protect the yield potential of a crop when grown in the presence of insect pests. To “control” insects may or may not mean killing the insects, although it preferably means killing the insects.

As used herein, the term “corn” is synonymous with the term “maize” or “Zea mays.”

As used herein, the terms “cross” or “crossed” refer to the fusion of gametes via pollination to produce progeny (e.g., cells, seeds or plants). The term encompasses both sexual crosses (the pollination of one plant by another) and selfing (self-pollination, e.g., when the pollen and ovule are from the same plant). The term “crossing” refers to the act of fusing gametes via pollination to produce progeny.

“Exon” refers to a section of DNA which carries the coding sequence for a protein or part of it. Exons are separated by intervening, non-coding sequences (introns). For purposes of the present invention, the definition of the term “exon” includes modifications to the nucleotide sequence of an exon derived from a target gene, provided the modified exon does not significantly reduce the activity of its associated 5′ regulatory sequence.

“Expression cassette” as used herein means a nucleic acid sequence capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The expression cassette comprising the nucleotide sequence of interest may have at least one of its components heterologous with respect to at least one of its other components. The expression cassette may also be one that is naturally occurring but has been obtained in a recombinant form useful for heterologous expression. Such usage of an expression cassette makes it so it is not naturally occurring in the cell into which it has been introduced. Typically, however, the expression cassette is heterologous with respect to the host, i.e., the particular nucleic acid sequence of the expression cassette does not occur naturally in the host cell and must have been introduced into the host cell or an ancestor of the host cell by a transformation process. The expression of the nucleotide sequence in the expression cassette may be under the control of a constitutive promoter or of an inducible promoter that initiates transcription only when the host cell is exposed to some particular external stimulus. In the case of a multicellular organism, such as a plant, the promoter can also be specific to a particular tissue, or organ, or stage of development.

An expression cassette also can optionally include a transcriptional and/or translational termination region (i.e., termination region) that is functional in plants. A variety of transcriptional terminators are available for use in expression cassettes and are responsible for the termination of transcription beyond the heterologous nucleotide sequence of interest and correct mRNA polyadenylation. The termination region may be native to the transcriptional initiation region, may be native to the operably linked nucleotide sequence of interest, may be native to the plant host, or may be derived from another source (i.e., foreign or heterologous to the promoter, the nucleotide sequence of interest, the plant host, or any combination thereof). Appropriate transcriptional terminators include, but are not limited to, the CAMV 35S terminator, the tml terminator, the nopaline synthase terminator and/or the pea rbcs E9 terminator. These can be used in both monocotyledons and dicotyledons. In addition, a coding sequence's native transcription terminator can be used. Any available terminator known to function in plants can be used in the context of the invention.

The term “expression” when used with reference to a polynucleotide, such as a gene, open reading frame (ORF) or portion thereof, or a transgene in plants, refers to the process of converting genetic information encoded in a gene into RNA (e.g., mRNA, rRNA, tRNA, or snRNA) through “transcription” of the gene (i.e., via the enzymatic action of an RNA polymerase), and into protein where applicable (e.g. if a gene encodes a protein), through “translation” of mRNA. Gene expression can be regulated at many stages in the process. For example, in the case of antisense or dsRNA constructs, respectively, expression may refer to the transcription of the antisense RNA only or the dsRNA only. In some embodiments of the invention, “expression” refers to the transcription and stable accumulation of sense (mRNA) or functional RNA. In some embodiments of the invention, “expression” refers to the production of protein.

As used herein, the term “functionally transcribed” means that the expression of a regulatory polynucleotide of the invention produces a protein of interest in a quantity that confers upon the tissue in which the protein is produced a phenotype for its intended purpose. For example, without limitation, a polynucleotide encoding an insecticidal protein, that is “functionally transcribed” in leaf tissue of a transgenic plant, for example a transgenic maize plant, produces a quantity of the insecticidal protein that is toxic to an insect pest that feeds on the leaf tissue in which the insecticidal protein is produced. Alternatively, the term “functionally transcribed” means that the quantity of the protein produced in any tissue of a transgenic plant is at least 50 ng/mg total soluble protein (TSP), or at least 100 ng/mg TSP, or at least 500 ng/mg TSP, or at least 800 ng/mg TSP, or at least 1000 ng/mg TSP, or at least 2000 ng/mg TSP or at least 3000 ng/mg TSP. In some embodiments of the invention, a polynucleotide operably linked to a promoter of the invention may be “functionally transcribed” in non-pollen tissues and transcribed at very low levels in pollen tissue of a transgenic plant and therefore produce a very low quantity of protein in the pollen tissue. Such very low quantity of protein may range from about 1 ng/mg TSP to about 15 ng/mg TSP. Such low expression levels does not mean that the polynucleotide is “functionally transcribed” in pollen, particularly compared to other non-pollen tissues in the same transgenic plant. In other embodiments, the level of protein produced in the non-pollen tissue of a transgenic plant, for example a transgenic maize plant, is at least 100-fold higher than in pollen tissue in the same transgenic plant, or at least 200-fold higher, or at least 300-fold higher, or at least 400-fold higher, or at least 500-fold higher, or at least 600-fold higher, or at least 700-fold higher, or at least 800-fold higher, or at least 900-fold higher or at least 1000-fold higher than in pollen tissue of the same transgenic plant.

A “gene” is a defined region that is located within a genome and comprises a coding nucleic acid sequence and typically also comprises other, primarily regulatory, nucleic acids responsible for the control of the expression, that is to say the transcription and translation, of the coding portion. A gene may also comprise other 5′ and 3′ untranslated sequences and termination sequences. Further elements that may be present are, for example, introns. The regulatory nucleic acid sequence of the gene may not normally be operatively linked to the associated nucleic acid sequence as found in nature and thus would be a chimeric gene.

“Gene of interest” refers to any nucleic acid molecule which, when transferred to a plant, confers upon the plant a desired trait such as antibiotic resistance, virus resistance, insect resistance, disease resistance, or resistance to other pests, herbicide tolerance, abiotic stress tolerance, male sterility, modified fatty acid metabolism, modified carbohydrate metabolism, improved nutritional value, improved performance in an industrial process or altered reproductive capability. The “gene of interest” may also be one that is transferred to plants for the production of commercially valuable enzymes or metabolites in the plant.

A “heterologous” nucleic acid sequence or nucleic acid molecule is a nucleic acid sequence or nucleic acid molecule not naturally associated with a host cell into which it is introduced, including non-naturally occurring multiple copies of a naturally occurring nucleic acid sequence. A heterologous nucleic acid sequence or nucleic acid molecule may comprise a chimeric sequence such as a chimeric expression cassette, where the promoter and the coding region are derived from multiple source organisms. The promoter sequence may be a constitutive promoter sequence, a tissue-specific promoter sequence, a chemically-inducible promoter sequence, a wound-inducible promoter sequence, a stress-inducible promoter sequence, or a developmental stage-specific promoter sequence.

A “homologous” nucleic acid sequence is a nucleic acid sequence naturally associated with a host cell into which it is introduced.

The term “identity” or “identical” or “substantially identical,” in the context of two nucleic acid or amino acid sequences, refers to two or more sequences or subsequences that have at least 60%, preferably at least 80%, more preferably 90%, even more preferably 95%, and most preferably at least 99% nucleotide or amino acid residue identity, when compared and aligned for maximum correspondence, as measured using one of the following sequence comparison algorithms or by visual inspection. Preferably, the substantial identity exists over a region of the sequences that is at least about 50 residues or bases in length, more preferably over a region of at least about 100 residues or bases, and most preferably the sequences are substantially identical over at least about 150 residues or bases. In an especially preferred embodiment, the sequences are substantially identical over the entire length of the coding regions. Furthermore, substantially identical nucleic acid or amino acid sequences perform substantially the same function.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2: 482 (1981), by the homology alignment algorithm of Needleman & Wunsch, J. Mol. Biol. 48: 443 (1970), by the search for similarity method of Pearson & Lipman, Proc. Nat'l. Acad Sci. USA 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Ausubel et al., infra).

One example of an algorithm that is suitable for determining percent sequence identity and sequence similarity is the BLAST algorithm, which is described in Altschul et al., J. Mol. Biol. 215: 403-410 (1990). Software for performing BLAST analyses is publicly available through the National Center for Biotechnology Information (National Center for Biotechnology Information, U.S. National Library of Medicine, 8600 Rockville Pike, Bethesda, MD 20894 USA). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., 1990). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad Sci. USA 89: 10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90: 5873-5787 (1993)). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

Another indication that two nucleic acid sequences are substantially identical is that the two molecules hybridize to each other under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. An extensive guide to the hybridization of nucleic acids is found in Tijssen (1993) Laboratory Techniques in Biochemistry and Molecular Biology-Hybridization with Nucleic Acid Probes part I chapter 2 “Overview of principles of hybridization and the strategy of nucleic acid probe assays” Elsevier, New York. Generally, highly stringent hybridization and wash conditions are selected to be about 5° C. lower than the thermal melting point (Tₘ) for the specific sequence at a defined ionic strength and pH. Typically, under “stringent conditions” a probe will hybridize to its target subsequence, but not to other sequences.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids which have more than 100 complementary residues on a filter in a Southern or northern blot is 50% formamide with 1 mg of heparin at 42° C., with the hybridization being carried out overnight. An example of highly stringent wash conditions is 0.15M NaCl at 72° C. for about 15 minutes. An example of stringent wash conditions is a 0.2× SSC wash at 65° C. for 15 minutes (see, Sambrook, infra, for a description of SSC buffer). Often, a high stringency wash is preceded by a low stringency wash to remove background probe signal. An example medium stringency wash for a duplex of, e.g., more than 100 nucleotides, is 1× SSC at 45° C. for 15 minutes. An example low stringency wash for a duplex of, e.g., more than 100 nucleotides, is 4-6× SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

The following are examples of sets of hybridization/wash conditions that may be used to clone homologous nucleotide sequences that are substantially identical to reference nucleotide sequences of the present invention: a reference nucleotide sequence preferably hybridizes to the reference nucleotide sequence in 7% sodium dodecyl sulfate (SDS), 0.5 M NaPO₄, 1 mM EDTA at 50° C. with washing in 2× SSC, 0.1% SDS at 50° C., more desirably in 7% sodium dodecyl sulfate (SDS), 0.5 M NaPO₄, 1 mM EDTA at 50° C. with washing in 1× SSC, 0.1% SDS at 50° C., more desirably still in 7% sodium dodecyl sulfate (SDS), 0.5 M NaPO₄, 1 mM EDTA at 50° C. with washing in 0.5× SSC, 0.1% SDS at 50° C., preferably in 7% sodium dodecyl sulfate (SDS), 0.5 M NaPO₄, 1 mM EDTA at 50° C. with washing in 0.1× SSC, 0.1% SDS at 50° C., more preferably in 7% sodium dodecyl sulfate (SDS), 0.5 M NaPO₄, 1 mM EDTA at 50° C. with washing in 0.1× SSC, 0.1% SDS at 65° C.

A further indication that two nucleic acid sequences or proteins are substantially identical is that the protein encoded by the first nucleic acid is immunologically cross reactive with, or specifically binds to, the protein encoded by the second nucleic acid. Thus, a protein is typically substantially identical to a second protein, for example, where the two proteins differ only by conservative substitutions.

“Intron” refers to an intervening section of DNA which occurs almost exclusively within a eukaryotic gene, but which is not translated to amino acid sequences in the gene product. The introns are removed from the pre-mature mRNA through a process called splicing, which leaves the exons untouched, to form an mRNA. For purposes of the present invention, the definition of the term “intron” includes modifications to the nucleotide sequence of an intron derived from a target gene, provided the modified intron does not significantly reduce the activity of its associated 5′ regulatory sequence.

The term “isolated” nucleic acid molecule, polynucleotide or protein is a nucleic acid molecule, polynucleotide or protein that no longer exists in its natural environment. An isolated nucleic acid molecule, polynucleotide or protein of the invention may exist in a purified form or may exist in a recombinant host such as in a transgenic bacteria or a transgenic plant. Therefore, a claim to an “isolated” nucleic acid molecule, as enumerated herein, encompasses a nucleic acid molecule that is comprised within a transgenic plant genome.

A “nucleic acid molecule” or “nucleic acid sequence” is a segment of single-or double-stranded DNA or RNA that can be isolated from any source. In the context of the invention, the nucleic acid molecule is typically a segment of DNA. In some embodiments, the nucleic acid molecules of the invention are isolated nucleic acid molecules.

“Operably linked” refers to the association of polynucleotides on a single nucleic acid fragment so that the function of one affects the function of the other. For example, a promoter is operably linked with a coding polynucleotide or functional RNA when it is capable of affecting the expression of that coding polynucleotide or functional RNA (i.e., that the coding polynucleotide or functional RNA is under the transcriptional control of the promoter). Coding polynucleotides in sense or antisense orientation can be operably linked to regulatory polynucleotides.

The terms “protein,” “peptide” and “polypeptide” are used interchangeably herein.

A “plant” is any plant at any stage of development, particularly a seed plant.

A “plant cell” is a structural and physiological unit of a plant, comprising a protoplast and a cell wall. The plant cell may be in the form of an isolated single cell or a cultured cell, or as a part of a higher organized unit such as, for example, plant tissue, a plant organ, or a whole plant.

“Plant cell culture” means cultures of plant units such as, for example, protoplasts, cell culture cells, cells in plant tissues, pollen, pollen tubes, ovules, embryo sacs, zygotes and embryos at various stages of development.

“Plant material” refers to leaves, stems, roots, flowers or flower parts, fruits, pollen, egg cells, zygotes, seeds, cuttings, cell or tissue cultures, or any other part or product of a plant.

A “plant organ” is a distinct and visibly structured and differentiated part of a plant such as a root, stem, leaf, flower bud, or embryo.

“Plant tissue” as used herein means a group of plant cells organized into a structural and functional unit. Any tissue of a plant in planta or in culture is included. This term includes, but is not limited to, whole plants, plant organs, plant seeds, tissue culture and any groups of plant cells organized into structural and/or functional units. The use of this term in conjunction with, or in the absence of, any specific type of plant tissue as listed above or otherwise embraced by this definition is not intended to be exclusive of any other type of plant tissue. For example, the “tapetum” is a tissue within the sporangium, especially the anther, of corn plants that provides nutrition for growing spores.

A “polynucleotide” refers to a polymer composed of many nucleotide monomers covalently bonded in a chain. Such “polynucleotides” includes DNA, RNA, modified oligo nucleotides (e.g., oligonucleotides comprising bases that are not typical to biological RNA or DNA, such as 2′-O-methylated oligonucleotides), and the like. In some embodiments, a nucleic acid or polynucleotide can be single-stranded, double-stranded, multi-stranded, or combinations thereof. Unless otherwise indicated, a particular nucleic acid or polynucleotide of the present invention optionally comprises or encodes complementary polynucleotides, in addition to any polynucleotide explicitly indicated.

“Polynucleotide of interest” refers to any polynucleotide which, when transferred to an organism, e.g., a plant, confers upon the organism a desired characteristic such as insect resistance, disease resistance, herbicide tolerance, antibiotic resistance, improved nutritional value, improved performance in an industrial process, production of commercially valuable enzymes or metabolites or altered reproductive capability.

A “promoter” is an untranslated DNA sequence upstream of the coding region that contains the binding site for RNA polymerase and initiates transcription of the DNA. The promoter region may also include other elements that act as regulators of gene expression.

“Preferred expression” is the expression of gene products that are preferably expressed at a higher level in one or a few plant tissues (spatial limitation) and/or to one or a few plant developmental stages (temporal limitation) while in other tissues/developmental stages there is a relatively low level of expression.

As used herein, the term “recombinant” refers to a form of nucleic acid (e.g., DNA or RNA) or protein or an organism that would not normally be found in nature and as such was created by human intervention. As used herein, a “recombinant nucleic acid molecule” is a nucleic acid molecule comprising a combination of polynucleotides that would not naturally occur together and is the result of human intervention, e.g., a nucleic acid molecule that is comprised of a combination of at least two polynucleotides heterologous to each other, or a nucleic acid molecule that is artificially synthesized, for example, a polynucleotide synthesize using an assembled nucleotide sequence, and comprises a polynucleotide that deviates from the polynucleotide that would normally exist in nature, or a nucleic acid molecule that comprises a transgene artificially incorporated into a host cell's genomic DNA and the associated flanking DNA of the host cell's genome. Another example of a recombinant nucleic acid molecule is a DNA molecule resulting from the insertion of a transgene into a plant's genomic DNA, which may ultimately result in the expression of a recombinant RNA or protein molecule in that organism. As used herein, a “recombinant plant” is a plant that would not normally exist in nature, is the result of human intervention, and contains a transgene or heterologous nucleic acid molecule incorporated into its genome. As a result of such genomic alteration, the recombinant plant is distinctly different from the related wild-type plant.

A “regulatory sequence” or “regulatory element,” and the like, is understood herein to refer to a nucleotide sequence that controls the expression of an operably associated coding sequence by providing the recognition for RNA polymerase and other factors required for proper transcription and is typically located upstream (5′) to its coding sequence. “Regulatory sequences” include 5′ regulatory sequences located proximal and more distal elements upstream of the associated coding region, which influence the transcription, RNA processing or stability, or translation of the associated coding sequence. “Regulatory sequences” may further include 3′ sequences, including 3′ non-translated and/or 3′ non-transcribed sequences, located downstream of the associated coding region, and can include a transcription termination site, i.e. terminators. “Regulatory sequences” may include enhancers, promoters, untranslated leader sequences, introns, and polyadenylation signal sequences. They include natural and synthetic sequences as well as sequences that may be a combination of synthetic and natural sequences. An “enhancer” is a DNA sequence that can stimulate promoter activity and may be an innate element of the promoter or a heterologous element inserted to enhance the level or tissue specificity of a promoter. It is capable of operating in both orientations (normal or flipped), and is capable of functioning even when moved either upstream or downstream from the promoter. Examples of enhancers include, among others, a figwort mosaic virus enhancer (eFMV) and a cauliflower mosaic virus enhancer (e35S). The meaning of the term “regulatory sequences” includes “transcription initiation” or “promoter” sequences and “promoter regulatory sequences.” These terms may herein be used interchangeably.

“Regulatory elements” refer to sequences involved in controlling the expression of a nucleotide sequence. Regulatory elements comprise a promoter operably linked to the nucleotide sequence of interest and termination signals. They also typically encompass sequences required for proper translation of the nucleotide sequence.

“Specific expression” is the expression of gene products that is limited to one or a few plant tissues (spatial limitation) and/or to one or a few plant developmental stages (temporal limitation).

“Tissue-specific promoter” refers to regulated promoters that are not expressed in all plant cells but only in one or more cell types in specific organs (such as leaves, roots or seeds), specific tissues (such as embryo or cotyledon), or specific cell types (such as leaf parenchyma or seed storage cells). These also include promoters that are temporally regulated, such as in early or late embryogenesis, during fruit ripening in developing seeds or fruit, in fully differentiated leaf, or at the onset of senescence.

A “transcriptional cassette” will comprise in the 5′-3′ direction of transcription, a transcriptional and translational initiation region, a DNA sequence of interest, and a transcriptional and translational termination region functional in plants. The termination region may be native with the transcriptional initiation region, may be native with the DNA sequence of interest, or may be derived from another source.

The “transcription initiation site” is the position surrounding the first nucleotide that is part of the transcribed sequence, which is also defined as position +1. With respect to this site all other sequences of the gene and its controlling regions are numbered. Downstream sequences (i.e. further protein encoding sequences in the 3′ direction) are denominated positive, while upstream sequences (mostly of the controlling regions in the 5′ direction) are denominated negative.

“Transient transformation” in the context of a polynucleotide means that a polynucleotide is introduced into the cell and does not integrate into the genome of the cell. Transient transformation may be detected by, for example, an enzyme-linked immunosorbent assay (ELISA) or Western blot, which can detect the presence of a peptide or polypeptide encoded by one or more nucleic acid molecules introduced into an organism. Stable transformation of a cell can be detected by, for example, a Southern blot hybridization assay of genomic DNA of the cell with nucleic acid sequences which specifically hybridize with a nucleotide sequence of a nucleic acid molecule introduced into an organism (e.g., a plant). Stable transformation of a cell can be detected by, for example, a Northern blot hybridization assay of RNA of the cell with nucleic acid sequences which specifically hybridize with a nucleotide sequence of a nucleic acid molecule introduced into a plant or other organism. Stable transformation of a cell can also be detected by, e.g., a polymerase chain reaction (PCR) or other amplification reaction as are well known in the art, employing specific primer sequences that hybridize with target sequence(s) of a nucleic acid molecule, resulting in amplification of the target sequence(s), which can be detected according to standard methods. Transformation can also be detected by direct sequencing and/or hybridization protocols well known in the art.

“Transformation” is a process for introducing heterologous nucleic acid into a host cell or organism. In particular embodiments, “transformation” means the stable integration of a DNA molecule into the genome (nuclear or plastid) of an organism of interest.

“Transformed/transgenic/recombinant” refer to a host organism such as a bacterium or a plant into which a heterologous nucleic acid molecule has been introduced. The nucleic acid molecule can be stably integrated into the genome of the host or the nucleic acid molecule can also be present as an extrachromosomal molecule. Such an extrachromosomal molecule can be auto-replicating. Transformed cells, tissues, or plants are understood to encompass not only the end product of a transformation process, but also transgenic progeny thereof. A “non-transformed”, “non-transgenic”, or “non-recombinant” host refers to a wild-type organism, e.g., a bacterium or plant, which does not contain the heterologous nucleic acid molecule.

“Vector” is defined to include, inter alia, any plasmid, cosmid, phage or Agrobacterium binary vector in double or single stranded linear or circular form which may or may not be self-transmissible or mobilizable, and which can transform prokaryotic or eukaryotic host either by integration into the cellular genome or exist extrachromosomally (e.g. autonomous replicating plasmid with an origin of replication). Specifically included are shuttle vectors by which is meant a DNA vehicle capable, naturally or by design, of replication in two different host organisms, which may be selected from actinomycetes and related species, bacteria and eukaryotic (e.g. higher plant, mammalian, yeast or fungal cells).

The invention relates generally to regulatory polynucleotides at least part of which have a transcription initiation function directing expression of an operably linked protein encoding polynucleotide to essentially all plant tissues, but essentially excluding expression in male reproductive tissues such as pollen and/or tassel tissue. The regulatory polynucleotides can be used in chimeric constructs, expression cassettes, recombinant vectors and the like to drive expression of a protein of interest, for example an insecticidal protein, in plant tissues that target pests normally feed on, and eliminate or reduce accumulation of the insecticidal protein in tissues not typically feed upon by pest insects or in those tissue that may be compromised by the insecticidal protein, such pollen or tassel.

Promoter sequences are obtained by cloning the genomic sequences that are homologous to the cDNA sequences expressed in tissues other than pollen, i.e. “non-pollen” cDNA sequences. Genomic sequences may be obtained by hybridization methods or by using PCR methods to extend the sequence in either the 5′ or 3′ direction from the known sequence (sometimes referred to as “genome walking”). For example, to obtain genomic sequences 5′ to the known sequence of the cDNA, primers are made to the sequence near the 5′ end of the cDNA. A genomic library is constructed with the 5′ end of each genomic DNA sequence ligated to a short oligonucleotide adapter. PCR with a primer hybridizing to the adapter sequence and a 5′ primer of a non-pollen cDNA sequence allows amplification of a genomic sequence residing 5′ to the homologous sequence of the non-pollen sequence. DNA sequences obtained from genome walking are sequenced and if additional 5′ regions are desired, the process is repeated with primers now at the 5′ end of the longest obtained clone. Genomic sequences homologous to non-pollen cDNA sequences are also obtained by hybridization under high stringency conditions. High stringency conditions select for hybridization of a probe made from a non-pollen cDNA sequence to hybridize to its homologous sequence in the genomic DNA. The genomic DNA is comprised in a genomic DNA library of 5-20 kb maize genomic DNA sequences in a lambda phage vector. Genomic clones that hybridize with the non-pollen cDNA are isolated and sequenced.

The promoters of the invention are located in the 5′-region of a pollen-minus cDNA sequence immediately upstream of the coding sequence. The size of the regulatory region is preferably in a range of between about 2 kb to 8 kb and comprises a 5′-non-transcribed sequence, particularly a 5′-non-transcribed sequence and a 5′-UTR and all or part of a nucleotide sequence representing a first intron. Promoters exemplified herein are set forth in SEQ ID NOS:1-11. The regulatory sequences of the invention may further comprise part of 3′-sequence that begins just past a coding sequence of a pollen-minus cDNA translation stop codon including transcribed but not translated sequence (UTR) and non-transcribed sequence that functions as the transcriptional terminator and a poly-adenylation signal. In particular, the 3′-sequence is in a range of between about 1.0 kb and about 2.5 kb. Terminators exemplified herein are set forth in SEQ ID NOS:12-20.

The genomic clones may include intron sequences, not found in the mRNA or the cDNA clones. The genomic sequences may additionally comprise 5′ untranslated sequences, 3′ untranslated sequences, and 5′ and 3′ regulatory sequences. Promoter sequences are found within the genomic sequence 5′ to the cDNA sequence. Genomic sequences are cloned which are homologous to the pollen-minus cDNA sequences. Sequences that are 5′ to the sequence homologous to the cDNA sequence are herein referred to as the 5′ flanking region which comprises the promoter region.

In some embodiments, the invention provides a chimeric DNA construct that comprises an insecticidal protein-coding sequence operably linked to a regulatory sequence, at least part of which has a transcription initiation function directing expression of the encoded insecticidal protein to essentially all tissues of the plant with the exception of male reproductive tissues, such as pollen and/or tassel, such that little or no expression product is present is the pollen and/or tassel tissues to any significant extent. Nucleic acid sequences of the invention can be provided as DNA or as RNA, as specified; disclosure of one necessarily defines the other, as is known to one of ordinary skill in the art. Furthermore, disclosure of a given nucleic acid sequence necessarily defines the exact complement of that sequence, as is known to one of ordinary skill in the art.

In some embodiments, the invention provides an expression cassette comprising a promoter comprising a nucleotide sequence a) having at least 95% identity to at least 99% identity to any of SEQ ID NOS:1-11; or b) of any of SEQ ID NOS:1-11, operably linked to a heterologous polynucleotide of interest, which is operably linked to a 3′-untranslated region including a polyadenylation signal, wherein the heterologous polynucleotide is functionally transcribed in a tissue of a transgenic plant that is not pollen. In other embodiments, the heterologous polynucleotide encodes an insecticidal protein or a double stranded RNA (dsRNA). In other embodiments, the insecticidal protein is a Cry protein or a Vip3 protein.

In still other embodiments, the insecticidal Cry protein is selected from the group consisting of Cry1Aa, Cry1Ab, Cry1Ac, Cry1Ad, Cry1Ae, Cry1Af, Cry1Ag, Cry1Ah, Cry1Ai, Cry1Aj, Cry1Ba, Cry1Bb, Cry1Bc, Cry1Bd, Cry1Be, Cry1Bf, Cry1Bg, Cry1Bh, Cry1Bi, Cry1Ca, Cry1Cb, Cry1Da, Cry1Db, Cry1Dc, Cry1Dd, Cry1Ea, Cry1Eb, Cry1Fa, Cry1Fb, Cry1Ga, Cry1Gb, Cry1Gc, Cry1Ha, Cry1Hb, Cry1Hc, Cry1Ia, Cry1Ib, Cry1Ic, Cry1Id, Cry1Ie, Cry1If, Cry1Ig, Cry1Ja, Cry1Jb, Cry1Jc, Cry1Jd, Cry1Ka, Cry1La, Cry1Ma, Cry1Na, Cry1Nb, Cry2Aa, Cry2Ab, Cry2Ac, Cry2Ad, Cry2Ae, Cry2Af, Cry2Ag, Cry2Ah, Cry2Ai, Cry2Aj, Cry2Ak, Cry2Al, Cry2Ba, Cry3Aa, Cry3Ba, Cry3Bb, Cry3Ca, Cry4Aa, Cry4Ba, Cry4Ca, Cry4Cb, Cry4Cc, Cry5Aa, Cry5Ab, Cry5Ac, Cry5Ad, Cry5Ba, Cry5Ca, Cry5Da, Cry5Ea, Cry6Aa, Cry6Ba, Cry7Aa, Cry7Ab, Cry7Ac, Cry7Ba, Cry7Bb, Cry7Ca, Cry7Cb, Cry7Da, Cry7Ea, Cry7Fa, Cry7Fb, Cry7Ga, Cry7Gb, Cry7Gc, Cry7Gd, Cry7Ha, Cry7Ia, Cry7Ja, Cry7Ka, Cry7Kb, Cry7La, Cry8Aa, Cry8Ab, Cry8Ac, Cry8Ad, Cry8Ba, Cry8Bb, Cry8Bc, Cry8Ca, Cry8Da, Cry8 Db, Cry8Ea, Cry8Fa, Cry8Ga, Cry8Ha, Cry8Ia, Cry8Ib, Cry8Ja, Cry8Ka, Cry8Kb, Cry8La, Cry8Ma, Cry8Na, Cry8Pa, Cry8Qa, Cry8Ra, Cry8Sa, Cry8Ta, Cry9Aa, Cry9Ba, Cry9Bb, Cry9Ca, Cry9Da, Cry9 Db, Cry9Dc, Cry9Ea, Cry9Eb, Cry9Ec, Cry9Ed, Cry9Ee, Cry9Fa, Cry9Ga, Cry10Aa, Cry11Aa, Cry11Ba, Cry11Bb, Cry12Aa, Cry13Aa, Cry14Aa, Cry14Ab, Cry15Aa, Cry16Aa, Cry17Aa, Cry18Aa, Cry18Ba, Cry18Ca, Cry19Aa, Cry19Ba, Cry19Ca, Cry20Aa, Cry20Ba, Cry21Aa, Cry21Ba, Cry21Ca, Cry21Da, Cry21Ea, Cry21Fa, Cry21Ga, Cry21Ha, Cry22Aa, Cry22Ab, Cry22Ba, Cry22Bb, Cry23 Aa, Cry24Aa, Cry24Ba, Cry24Ca, Cry25Aa, Cry26Aa, Cry27Aa, Cry28Aa, Cry29Aa, Cry29Ba, Cry30Aa, Cry30Ba, Cry30Ca, Cry30Da, Cry30 Db, Cry30Ea, Cry30Fa, Cry30Ga,Cry31Aa, Cry31Ab, Cry31Ac, Cry31Ad, Cry32Aa, Cry32Ab, Cry32Ba, Cry32Ca, Cry32Cb, Cry32Da, Cry32Ea, Cry32Eb, Cry32Fa, Cry32Ga, Cry32Ha, Cry32Hb, Cry32Ia, Cry32Ja, Cry32Ka, Cry32La, Cry32Ma, Cry32Mb, Cry32Na, Cry32Oa, Cry32Pa, Cry32Qa, Cry32Ra, Cry32Sa, Cry32Ta, Cry32Ua, Cry33Aa, Cry34Aa, Cry34Ab, Cry34Ac, Cry34Ba, Cry35Aa, Cry35Ab, Cry35Ac, Cry35Ba, Cry36Aa, Cry37Aa, Cry38Aa, Cry39Aa, Cry40Aa, Cry40Ba, Cry40Ca, Cry40Da, Cry41Aa, Cry41Ab, Cry41Ba, Cry42Aa, Cry43Aa, Cry43Ba, Cry43Ca, Cry43Cb, Cry43Cc, Cry44Aa, Cry45Aa, Cry46Aa Cry46Ab, Cry47Aa, Cry48Aa, Cry48Ab, Cry49Aa, Cry49Ab, Cry50Aa, Cry50Ba, Cry51Aa, Cry52Aa, Cry52Ba, Cry53Aa, Cry53Ab, Cry54Aa, Cry54Ab, Cry54Ba, Cry55Aa, Cry56Aa, Cry57Aa, Cry57Ab, Cry58Aa, Cry59Aa, Cry59Ba, Cry60Aa, Cry60Ba, Cry61Aa, Cry62Aa, Cry63Aa, Cry64Aa, Cry65Aa, Cry66Aa, Cry67Aa, Cry68Aa, Cry69Aa, Cry69Ab, Cry70Aa, Cry70Ba, Cry70Bb, Cry71Aa, Cry72Aa and Cry73Aa. In still other embodiments, the Cry protein is a Cry1 protein. In other embodiments, the Cry1 protein is a Cry1A protein. In still other embodiments, the Cry1A protein is a Cry1Ab or a Cry1Ai protein.

In other embodiments, the Vip3 insecticidal protein encoded by a heterologous operably linked to a promoter of the invention is selected from the group consisting of Vip3Aa1, Vip3Aa2, Vip3Aa3, Vip3Aa4, Vip3Aa5, Vip3Aa6, Vip3Aa7, Vip3Aa8, Vip3 Aa9, Vip3Aa10, Vip3Aa11, Vip3Aa12, Vip3Aa13, Vip3Aa14, Vip3Aa15, Vip3 Aa16, Vip3Aa17, Vip3Aa18, Vip3Aa19, Vip3Aa20, Vip3Aa21, Vip3Aa22, Vip3 Aa2, Vip3 Aa24, Vip3Aa25, Vip3 Aa26, Vip3 Aa27, Vip3Aa28, Vip3Aa29, Vip3 Aa30, Vip3 Aa31, Vip3 Aa32, Vip3 Aa33, Vip3 Aa34, Vip3 Aa35, Vip3Aa36, Vip3 Aa37, Vip3Aa38, Vip3Aa39, Vip3Aa40, Vip3 Aa41, Vip3 Aa42, Vip3Aa43, Vip3Aa44, Vip3Ab1, Vip3Ab2, Vip3Ac1, Vip3Ad1, Vip3Ad2, Vip3Ae1, Vip3Af1, Vip3 Af2, Vip3Af3, Vip3 Ag1, Vip3Ag2, Vip3Ag3 HM117633, Vip3Ag4, Vip3 Ag5, Vip3Ah1, Vip3Ba1, Vip3Ba2, Vip3Bb1, Vip3Bb2 and Vip3Bb3. In still other embodiments, the Vip3 protein is a Vip3A protein. In other embodiments, the Vip3A protein is a Vip3 Aa protein. In other embodiments, the Vip3 Aa protein is a Vip3Aa19 or a Vip3 Aa20 protein.

In some embodiments, the transgenic plant in which the heterologous polynucleotide of the invention is functionally transcribed is a monocot plant. In other embodiments, the monocot plant is maize plant. In other embodiments, the maize plant is an inbred maize plant or a hybrid maize plant. In some embodiments, the tissue of a maize plant in which the heterologous polynucleotide is functionally expressed is leaf, silk or husk.

In some embodiments, the 3′-untranslated region of an expression cassette of the invention comprises a terminator sequence selected from any of SEQ ID NOS:12-20. In still other embodiments, the expression cassette comprises SEQ ID NO:29.

In some embodiments, the invention provides a recombinant vector comprising an expression cassette of the invention. In other embodiments, the recombinant vector is a binary vector capable of functioning in multiple organisms. In other embodiments, the organism is a bacteria or a plant. In other embodiments, the binary vector comprises any of SEQ ID NOS:21-28.

In some embodiments, the invention provides a plant cell transformed with a recombinant vector or an expression cassette of the invention. In other embodiments, the recombinant vector or expression cassette is transiently expressed in the plant cell. In other embodiments, the recombinant vector or expression cassette of the invention is stably integrated into the genome of the plant cell. In other embodiments, the plant cell is a monocot plant cell. In still other embodiments, the monocot plant cell is a maize plant cell. In other embodiments, the expression cassette comprises a sequence of an expression cassette in any of SEQ ID NOS:21-28. In still other embodiments, the expression cassette comprise SEQ ID NO:29.

In some embodiments, the invention provides a transgenic plant comprising an expression cassette or a vector of the invention. In other embodiments, the expression cassette or vector or a portion thereof is stably integrated into the genome of the transgenic plant. In other embodiments, the transgenic plant is a monocot. In still other embodiments, the monocot transgenic plant is a transgenic maize plant. In other embodiments, the transgenic maize plant is an inbred maize plant or a hybrid maize plant. In other embodiments, the transgenic maize plant expresses a protein of interest at functional levels in any tissue that is not pollen. In still other embodiments, the tissue where the protein of interest is expressed at functional levels is leaf, silk or husk.

In other embodiments, the protein of interest that is expressed in a transgenic plant at functional levels in any tissue that is not pollen is a Cry protein or a Vip3 protein. In other embodiments, the Cry protein or the Vip3 protein is selected from a list of Cry and Vip3 proteins described above. In other embodiments, the Cry protein is a Cry1 protein. In other embodiments, the Cry1 protein is a Cry1A protein. In still other embodiments, the Cry1A protein is a Cry1Ab protein or a Cry1Ai protein. In still other embodiments, the Vip3 protein is a Vip3A protein. In other embodiments, the Vip3A protein is a Vip3Aa protein. In still other embodiments, the VIp3Aa protein is a Vip3Aa19 or a Vip3Aa20 protein.

In some embodiments, the invention provides a transgenic seed of any transgenic plant of the invention. In other embodiments, the transgenic seed comprises an expression cassette or a vector, or a portion thereof, of the invention. In other embodiments, the transgenic seed is a maize seed. In other embodiments, the maize seed functions as a maize propagation means. In other embodiments, the transgenic maize seed functions as harvested grain.

In some embodiments, the invention provides an isolated nucleic acid molecule comprising a nucleotide sequence selected from the group consisting of: a) a sequence set forth in any of SEQ ID NOS:1-20; b) a nucleotide sequence that hybridizes under high stringency conditions to a nucleotide sequence of a); and c) a nucleotide sequence comprising a fragment of a nucleotide sequence of a), wherein the fragment maintains the function of the nucleotide sequence of a); wherein the nucleic acid molecule initiates functional transcription or ends functional transcription of an operably linked polynucleotide of interest in a tissue of a transgenic plant that is not pollen.

In some embodiments, the polynucleotide of interest that is operably linked to the isolated nucleic acid molecule of the invention encodes an insecticidal protein or a double stranded RNA (dsRNA). In other embodiments, the insecticidal protein is a Cry protein or a Vip3 protein. In other embodiments, the Cry protein or the Vip3 protein is selected from a list of Cry and Vip3 proteins described above. In other embodiments, the Cry protein is a Cry1 protein. In other embodiments, the Cry1 protein is a Cry1A protein. In other embodiments, the Cry1A protein is a Cry1Ab or a Cry1Ai protein. In still other embodiments, the Vip3 protein is a Vip3A protein. In other embodiment, the Vip3A protein is a Viip3Aa protein. In other embodiments, the Vip3Aa protein is a Vip3 Aa19 or Vip3 Aa20 protein.

In some embodiments, an isolated nucleic acid molecule of the invention comprises a promoter nucleotide sequence that is set forth in SEQ ID NO:1. In other embodiments, an isolated nucleic acid molecule of the invention comprises a promoter nucleotide sequence that is set forth in SEQ ID NO:1 and a terminator nucleotide sequence that is set forth in SEQ ID NO:12.

In some embodiments, an isolated nucleic acid molecule of the invention comprises a promoter nucleotide sequence that is set forth in SEQ ID NO:2. In other embodiments, an isolated nucleic acid molecule of the invention comprises a promoter nucleotide sequence that is set forth in SEQ ID NO:2 and a terminator nucleotide sequence that is set forth in SEQ ID NO:13 or SEQ ID NO:14.

In some embodiments, an isolated nucleic acid molecule of the invention comprises a promoter nucleotide sequence that is set forth in SEQ ID NO:3. In other embodiments, an isolated nucleic acid molecule of the invention comprises a promoter nucleotide sequence that is set forth in SEQ ID NO:3 and a terminator nucleotide sequence that is set forth in SEQ ID NO:13 or SEQ ID NO:14.

In some embodiments, an isolated nucleic acid molecule of the invention comprises a promoter nucleotide sequence that is set forth in SEQ ID NO:4. In other embodiments, an isolated nucleic acid molecule of the invention comprises a promoter nucleotide sequence that is set forth in SEQ ID NO:4 and a terminator nucleotide sequence that is set forth in SEQ ID NO:13 or SEQ ID NO:14.

In some embodiments, an isolated nucleic acid molecule of the invention comprises a promoter nucleotide sequence that is set forth in SEQ ID NO:5 In other embodiments, an isolated nucleic acid molecule of the invention comprises a promoter nucleotide sequence that is set forth in SEQ ID NO:5 and a terminator nucleotide sequence that is set forth in SEQ ID NO:13 or SEQ ID NO:14.

In some embodiments, an isolated nucleic acid molecule of the invention comprises a promoter nucleotide sequence that is set forth in SEQ ID NO:6. In other embodiments, an isolated nucleic acid molecule of the invention comprises a promoter nucleotide sequence that is set forth in SEQ ID NO:6 and a terminator nucleotide sequence that is set forth in SEQ ID NO:15.

In some embodiments, an isolated nucleic acid molecule of the invention comprises a promoter nucleotide sequence that is set forth in SEQ ID NO:7. In other embodiments, an isolated nucleic acid molecule of the invention comprises a promoter nucleotide sequence that is set forth in SEQ ID NO:7 and a terminator nucleotide sequence that is set forth in SEQ ID NO:16.

In some embodiments, an isolated nucleic acid molecule of the invention comprises a promoter nucleotide sequence that is set forth in SEQ ID NO:8. In other embodiments, an isolated nucleic acid molecule of the invention comprises a promoter nucleotide sequence that is set forth in SEQ ID NO:8 and a terminator nucleotide sequence that is set forth in SEQ ID NO:17.

In some embodiments, an isolated nucleic acid molecule of the invention comprises a promoter nucleotide sequence that is set forth in SEQ ID NO:9. In other embodiments, an isolated nucleic acid molecule of the invention comprises a promoter nucleotide sequence that is set forth in SEQ ID NO:9 and a terminator nucleotide sequence that is set forth in SEQ ID NO:18.

In some embodiments, an isolated nucleic acid molecule of the invention comprises a promoter nucleotide sequence that is set forth in SEQ ID NO:10. In other embodiments, an isolated nucleic acid molecule of the invention comprises a promoter nucleotide sequence that is set forth in SEQ ID NO:10 and a terminator nucleotide sequence that is set forth in SEQ ID NO:19.

In some embodiments, an isolated nucleic acid molecule of the invention comprises a promoter nucleotide sequence that is set forth in SEQ ID NO:11. In other embodiments, an isolated nucleic acid molecule of the invention comprises a promoter nucleotide sequence that is set forth in SEQ ID NO:11 and a terminator nucleotide sequence that is set forth in SEQ ID NO:20.

In some embodiments, the isolated nucleic acid molecule of the invention initiates functional transcription or ends functional transcription of an operably linked polynucleotide of interest in any non-pollen tissue of a transgenic maize plant.

In some embodiments, the invention provides a method for expressing a protein or polynucleotide of interest in a transgenic plant or a transgenic plant cell comprising introducing into a plant or a plant cell an expression cassette or vector of the invention comprising a promoter operably linked to a heterologous polynucleotide that encodes the protein or polynucleotide of interest, wherein the promoter comprises any of SEQ ID NOS:1-11, and wherein the protein or polynucleotide of interest is functionally expressed in a tissue of the transgenic plant that is not pollen. In other embodiments, the expression cassette or vector also comprise a terminator sequence operably linked to the heterologous polynucleotide that comprises any of SEQ ID NOS:12-20.

In other embodiments of the method for expressing a protein or polynucleotide of interest in a transgenic plant or a transgenic plant cell, the protein of interest is an insecticidal protein or the polynucleotide of interest is a dsRNA that is insecticidal. In other embodiments, the insecticidal protein is a Cry protein or a Vip3 protein. In other embodiments, the Cry protein or the Vip3 protein is selected from a list of Cry and Vip3 proteins described above. In other embodiments, the Cry protein is a Cry1 protein. In still other embodiments, the Cry1 protein is a Cry1A protein. In other embodiments, the Cry1A protein is a Cry1Ab or a Cry1Ai protein. In still other embodiments, the Vip3 protein is a Vip3A protein. In other embodiment, the Vip3A protein is a Viip3Aa protein. In other embodiments, the Vip3Aa protein is a Vip3Aa19 or a Vip3Aa20 protein.

In other embodiments of the method for expressing a protein or polynucleotide of interest in a transgenic plant or a transgenic plant cell, the transgenic plant is a transgenic maize plant. In other embodiments, the transgenic maize plant is an inbred maize plant or a hybrid maize plant. In still other embodiments, the tissue where the protein of interest is expressed at functional levels in the transgenic maize plant is leaf, silk or husk.

In some embodiments of the invention, to further delineate the sequences required for expression in any tissue that is not pollen as well as those regulatory sequences that influence the overall level of expression, deletions in a pollen-minus promoter region may be made. Deletions are made in the 5′ flanking region of each pollen-minus genomic clone. In most promoters 500-1000 base pairs (bp) of 5′ flanking sequence are sufficient for promoter activity, including tissue-specific activity. Deletions of the 5′ flanking region can result in promoter regions of approximately 50 bp, 100 bp, 250 bp, 500 bp, 750 bp and 1000 bp or more. These promoter deletion sequences serve a two-fold purpose. The deletions allow the further mapping of regulatory sequences within the 5′ flanking sequence of each pollen-minus genomic clone. Additionally, the deletions provide a toolbox of promoter and regulatory sequences that vary in their expression levels and expression patterns thus providing additional flexibility in choosing promoter sequences for appropriate gene regulation.

It is also clear to one skilled in the art that mutations, insertions, deletions and/or substitutions of one or more nucleotides can be introduced into the nucleotide sequences of SEQ ID NOS:1-11 using methods known in the art. In addition, shuffling the sequences of the invention can provide new and varied nucleotide sequences. For example, SEQ ID NOS:3-5 are variants of SEQ ID NO:1 that have one or more substitutions, deletions or additions compared to SEQ ID NO:2.

To test for a function of variant DNA sequences according to the invention, such as deletion fragments of SEQ ID NOS:1-11, the sequence of interest is operably linked to a selectable or visible marker gene and expression of the marker gene is tested in transient expression assays with isolated tissues, such as leaf tissue, or cells or by stable transformation into plants. It is known to the skilled artisan that DNA sequences capable of driving expression of an associated coding sequence are built in a modular way. Accordingly, expression levels from shorter DNA fragments may be different than the one from the longest fragment and may be different from each other. For example, deletion of a down-regulating upstream element will lead to an increase in the expression levels of the associated coding sequence while deletion of an up-regulating element will decrease the expression levels of the associated coding sequence. It is also known to the skilled artisan that deletion of development-specific or a tissue-specific elements will lead to a temporally or spatially altered expression profile of the associated coding sequence.

In another embodiment of the invention, DNA and genomic DNA sequences homologous to SEQ ID NOS:1-11 may be isolated from other maize germplasm using either hybridization or PCR techniques well known in the art. The isolated sequences may be identical to SEQ ID NOS:1-11 or they may be substantially identical to SEQ ID NOS:1-11. It is not necessary for the sequences obtained from other maize germplasm to contain identical nucleotide sequences to be functionally identical to the sequences disclosed herein. Some nucleotide deletions, additions, and replacements may have no impact or only a minor impact on gene expression. A preferable isolated nucleic acid molecule, according to the present invention, comprises a nucleotide sequence that has at least 90%, or at least 91%, 92%, 93%, 94%, 95%, 96%, 97%, 98% or at least 99% identity to any one of the nucleotide sequences set forth in SEQ ID NOS:1-11. A more preferable isolated nucleic acid molecule comprises a nucleotide sequence that has at least 90% identity to any one of the nucleotide sequences set forth in SEQ ID NOS:1-11. An even more preferable isolated nucleic acid molecule comprises a nucleotide sequence that has at least 95% identity to any one of the nucleotide sequences set forth in SEQ ID NOS:1-11. An even more preferable isolated nucleic acid molecule comprises a nucleotide sequence that has at least 99% identity to any one of the nucleotide sequences set forth in SEQ ID NOS:1-11. The most preferable isolated nucleic acid molecule comprises any one of the nucleotide sequences set forth is SEQ ID NOS:1-11.

In other embodiments, cDNA and genomic DNA sequences may be cloned from other plants that represent homologues of the pollen-minus maize genes and promoters. These homologues allow one to obtain additional pollen-minus promoters useful for the regulation of multiple genes in plants tissues other than pollen. Hybridization using the maize cDNA and genomic sequences or portions thereof is used to screen for homologous or substantially identical sequences in other plant genomes. These sequences may comprise only a subset of the nucleotides of SEQ ID NOS:1-11. A preferable length of homology is 20 base pairs (bp) in length, more preferably, 50 bp in length, and most preferably at least 100 bp in length. In one embodiment of the present invention, a hybridization probe is prepared from any one SEQ ID NOS:1-11 or portions. Hybridization of such sequences may be carried out under high stringency conditions. Alternatively, low or moderate stringency conditions can be used to allow some mismatching in sequences so that lower degrees of similarity are detected (heterologous probing). Generally, a probe is less than about 1000 nucleotides in length, preferably less than 500 nucleotides in length.

In other embodiments of the invention, cDNA and genomic sequences are isolated by preparing primers comprising sequences within any one of SEQ ID NOS:1-11. The primers may be used in a PCR reaction with cDNA or genomic DNA from a plant to obtain homologous sequences or sequences with substantial identity to any one of SEQ ID NOS:1-11.

### Construction of Expression Cassettes

Expression cassettes are constructed comprising the 5′ flanking sequences of the pollen-minus genomic clones. In embodiments of the invention, the promoter region utilized in each expression cassette comprises the 5′ flanking region up to and including the start of translation. The start of translation is denoted by the first ATG of the open reading frame (ORF) found in the cDNA and the homologous genomic sequence. Thus, the promoter region may include 5′ untranslated leader sequence as well as the transcriptional start site, core promoter and additional regulatory elements. In other embodiments of the invention, expression cassettes are constructed comprising the 5′ flanking sequence of the pollen-minus genomic clones up to and including the transcriptional initiation site. The transcriptional initiation site may be defined by the first nucleotide of the longest cDNA clone obtained. Additionally, the transcriptional initiation site may be further defined by use of techniques well known in the art including RACE PCR, RNase protection mapping and primer extension analysis.

The expression cassettes may further comprise a transcriptional terminator, downstream (3′) to the promoter. A variety of transcriptional terminators are available for use in expression cassettes. The transcriptional terminator is responsible for the termination of transcription beyond the transgene and correct mRNA polyadenylation of the mRNA transcript. Appropriate transcriptional terminators are those that are known to function in plants and include the CaMV 35S terminator, the tml terminator, the nopaline synthase terminator and the pea rbcS E9 terminator. These can be used in both monocotyledons and dicotyledons. In addition, a gene's native transcription terminator may be used. For example, the 3′ flanking sequence comprising genomic sequence 3′ to the region homologous to a pollen-minus cDNA clone may be used. Such terminators exemplified herein include any of SEQ ID NOS:12-20.

In some embodiments of the invention a heterologous coding sequence, for example, an insecticidal coding sequence, a visible marker coding sequence, or a selectable marker coding sequence, is cloned between a promoter of the invention and transcriptional terminator whereby the heterologous coding sequence is operatively linked to the promoter and the transcriptional terminator is operatively linked to the heterologous coding sequence. Examples of visible markers useful for the invention include, but are not limited to, □-glucuronidase (GUS), Chloramphenicol Acetyl Transferase (CAT), Luciferase (LUC) and proteins with fluorescent properties, such as Green Fluorescent Protein (GFP) from Aequora victoria. In principle, many more proteins are suitable for this purpose, provided the protein does not interfere with essential plant functions. Further examples of heterologous coding sequences useful for the invention include, but are not limited to, antibiotic resistance, virus resistance, insect resistance, disease resistance, or resistance to other pests, herbicide tolerance, improved nutritional value, improved performance in an industrial process or altered reproductive capability. In other embodiments of the invention, a gene encoding for resistance to insects that feed on the tissues of plants that are not pollen is cloned between a promoter of the invention and a terminator known in the art or of the present invention. In another embodiment of the present invention a sequence encoding a functional RNA such as antisense RNA, a sense RNA for sense-suppression, or a double stranded RNA may also be cloned between the promoter and transcriptional terminator.

Numerous sequences have been found to enhance gene expression from within the transcriptional unit and these sequences can be used in conjunction with the promoters of this invention to increase their expression in transgenic plants. Various intron sequences have been shown to enhance expression, particularly in monocotyledonous cells. For example, a number of non-translated leader sequences derived from viruses are also known to enhance expression, and these are particularly effective in dicotyledonous cells. Specifically, leader sequences from Tobacco Mosaic Virus (TMV, the “W-sequence”), Maize Chlorotic Mottle Virus (MCMV), and Alfalfa Mosaic Virus (AMV) have been shown to be effective in enhancing expression (e.g. Gallie et al. Nucl. Acids Res. 15: 8693-8711 (1987); Skuzeski et al. Plant Molec. Biol. 15: 65-79 (1990)). A pollen-minus promoter of the invention may include, or be modified to include, one or more enhancer elements. In some embodiments, the promoter may include a plurality of enhancer elements. Promoters containing enhancer elements provide for higher levels of transcription in non-pollen tissues as compared to promoters that do not include them. Suitable enhancer elements for use in plants include the PCISV enhancer element (U.S. Pat. No. 5,850,019), the CaMV 35S enhancer element (U.S. Pat. Nos. 5,106,739 and 5,164,316) and the FMV enhancer element (Maiti et al. (1997) Transgenic Res. 6:143-156); the translation activator of the tobacco mosaic virus (TMV) described in Application WO87/07644, or of the tobacco etch virus (TEV) described by Carrington & Freed 1990, J. Virol. 64: 1590-1597, for example, or introns such as the adh1 intron of maize or intron 1 of rice actin. See also PCT WO96/23898, WO2012/021794, WO2012/021797, WO2011/084370, and WO2011/028914.

### Plant Transformation

Procedures for transforming plants are known in the art and are described throughout the literature. Non-limiting examples of methods for transformation of plants include transformation via bacterial-mediated nucleic acid delivery (e.g., via Agrobacterium), viral-mediated nucleic acid delivery, silicon carbide or nucleic acid whisker-mediated nucleic acid delivery, liposome mediated nucleic acid delivery, microinjection, microparticle bombardment, calcium-phosphate-mediated transformation, cyclodextrin-mediated transformation, electroporation, nanoparticle-mediated transformation, sonication, infiltration, PEG-mediated nucleic acid uptake, as well as any other electrical, chemical, physical (mechanical) or biological mechanism that results in the introduction of nucleic acid into the plant cell, including any combination thereof. General guides to various plant transformation methods known in the art include Miki et al. (“Procedures for Introducing Foreign DNA into Plants” in Methods in Plant Molecular Biology and Biotechnology, Glick, B. R. and Thompson, J. E., Eds. (CRC Press, Inc., Boca Raton, 1993), pages 67-88) and Rakowoczy-Trojanowska (Cell. Mol. Biol. Lett. 7:849-858 (2002)).

For Agrobacterium-mediated transformation, binary vectors or vectors carrying at least one T-DNA border sequence are suitable, whereas for direct gene transfer (e.g., particle bombardment and the like) any vector is suitable and linear DNA containing only the construction of interest can be used. In the case of direct gene transfer, transformation with a single DNA species or co-transformation can be used (Schocher et al., Biotechnology 4:1093-1096 (1986)). For both direct gene transfer and Agrobacterium-mediated transfer, transformation is usually (but not necessarily) undertaken with a selectable marker that may be a positive selection (Phosphomannose Isomerase; PMI), provide resistance to an antibiotic (kanamycin, hygromycin or methotrexate) or a herbicide (glyphosate or glufosinate). However, the choice of selectable marker is not critical to the invention.

Agrobacterium-mediated transformation is a commonly used method for transforming plants because of its high efficiency of transformation and because of its broad utility with many different species. Agrobacterium-mediated transformation typically involves transfer of the binary vector carrying the foreign DNA of interest to an appropriate Agrobacterium strain that may depend on the complement of vir genes carried by the host Agrobacterium strain either on a co-resident Ti plasmid or chromosomally (Uknes et al. (1993) Plant Cell 5:159-169). The transfer of the recombinant binary vector to Agrobacterium can be accomplished by a triparental mating procedure using Escherichia coli carrying the recombinant binary vector, a helper E. coli strain that carries a plasmid that is able to mobilize the recombinant binary vector to the target Agrobacterium strain. Alternatively, the recombinant binary vector can be transferred to Agrobacterium by nucleic acid transformation (Höfgen & Willmitzer (1988) Nucleic Acids Res. 16:9877).

Dicots as well as monocots may be transformed using Agrobacterium. Methods for Agrobacterium-mediated transformation of rice include well known methods for rice transformation, such as those described in any of the following: European patent application EP 1198985 A1, Aldemita and Hodges (Planta 199: 612-617, 1996); Chan et al. (Plant Mol Biol 22 (3): 491-506, 1993), Hiei et al. (Plant J 6 (2): 271-282, 1994), which disclosures are incorporated by reference herein as if fully set forth. In the case of corn transformation, the preferred method is as described in either Ishida et al. (Nat. Biotechnol 14(6): 745-50, 1996) or Frame et al. (Plant Physiol 129(1): 13-22, 2002), which disclosures are incorporated by reference herein as if fully set forth. Said methods are further described by way of example in B. Jenes et al., Techniques for Gene Transfer, in: Transgenic Plants, Vol. 1, Engineering and Utilization, eds. S. D. Kung and R. Wu, Academic Press (1993) 128-143 and in Potrykus Annu. Rev. Plant Physiol. Plant Molec. Biol. 42 (1991) 205-225). The nucleic acids or the construct to be expressed is preferably cloned into a vector, which is suitable for transforming Agrobacterium tumefaciens, for example pBin19 (Bevan et al., Nucl. Acids Res. 12 (1984) 8711). Agrobacteria transformed by such a vector can then be used in known manner for the transformation of plants, such as plants used as a model, like Arabidopsis or crop plants such as, by way of example, tobacco plants, for example by immersing bruised leaves or chopped leaves in an agrobacterial solution and then culturing them in suitable media. The transformation of plants by means of Agrobacterium tumefaciens is described, for example, by Hagen and Willmitzer in Nucl. Acid Res. (1988) 16, 9877 or is known inter alia from F. F. White, Vectors for Gene Transfer in Higher Plants; in Transgenic Plants, Vol. 1, Engineering and Utilization, eds. S. D. Kung and R. Wu, Academic Press, 1993, pp. 15-38.

Transformation of a plant by recombinant Agrobacterium usually involves co-cultivation of the Agrobacterium with explants from the plant and follows methods well known in the art. Transformed tissue is regenerated on selection medium carrying an antibiotic or herbicide resistance marker between the binary plasmid T-DNA borders.

As discussed previously, another method for transforming plants, plant parts and plant cells involves propelling inert or biologically active particles at plant tissues and cells. See, e.g., U.S. Pat. Nos. 4,945,050; 5,036,006 and 5,100,792. Generally, this method involves propelling inert or biologically active particles at the plant cells under conditions effective to penetrate the outer surface of the cell and afford incorporation within the interior thereof. When inert particles are utilized, the vector can be introduced into the cell by coating the particles with the vector containing the nucleic acid of interest. Alternatively, a cell or cells can be surrounded by the vector so that the vector is carried into the cell by the wake of the particle. Biologically active particles (e.g., a dried yeast cell, a dried bacterium or a bacteriophage, each containing one or more nucleic acids sought to be introduced) also can be propelled into plant tissue.

In other embodiments, a polynucleotide of the invention can be directly transformed into the plastid genome. A major advantage of plastid transformation is that plastids are generally capable of expressing bacterial genes without substantial modification, and plastids are capable of expressing multiple open reading frames under control of a single promoter. Plastid transformation technology is extensively described in U.S. Pat. Nos. 5,451,513, 5,545,817, and 5,545,818, in PCT application no. WO 95/16783, and in McBride et al. (1994) Proc. Nati. Acad. Sci. USA 91, 7301-7305. The basic technique for chloroplast transformation involves introducing regions of cloned plastid DNA flanking a selectable marker together with the gene of interest into a suitable target tissue, e.g., using biolistics or protoplast transformation (e.g., calcium chloride or PEG mediated transformation). The 1 to 1.5 kb flanking regions, termed targeting sequences, facilitate homologous recombination with the plastid genome and thus allow the replacement or modification of specific regions of the plastome. Initially, point mutations in the chloroplast 16S rRNA and rps12 genes conferring resistance to spectinomycin or streptomycin can be utilized as selectable markers for transformation (Svab, Z., Hajdukiewicz, P., and Maliga, P. (1990) Proc. Natl. Acad. Sci. USA 87, 8526-8530; Staub, J. M., and Maliga, P. (1992) Plant Cell 4, 39-45). The presence of cloning sites between these markers allows creation of a plastid targeting vector for introduction of foreign genes (Staub, J. M., and Maliga, P. (1993) EMBO J. 12, 601-606). Substantial increases in transformation frequency can be obtained by replacement of the recessive rRNA or r-protein antibiotic resistance genes with a dominant selectable marker, the bacterial aadA gene encoding the spectinomycin-cletoxifying enzyme aminoglycoside-3′-adenyltransf erase (Svab, Z., and Maliga, P. (1993) Proc. Natl. Acad. Sci. USA 90, 913-917). Previously, this marker had been used successfully for high-frequency transformation of the plastid genome of the green alga Chlamydomonas reinhardtii (Goldschmidt-Clermont, M. (1991) Nucl. Acids Res. 19:4083-4089). Other selectable markers useful for plastid transformation are known in the art and encompassed within the scope of the invention. Typically, approximately 15-20 cell division cycles following transformation are required to reach a homoplastidic state. Plastid expression, in which genes are inserted by homologous recombination into all of the several thousand copies of the circular plastid genome present in each plant cell, takes advantage of the enormous copy number advantage over nuclear-expressed genes to permit expression levels that can readily exceed 10% of the total soluble plant protein. In one embodiment, a polynucleotide of the invention can be inserted into a plastid-targeting vector and transformed into the plastid genome of a desired plant host. Thus, plants homoplastic for plastid genomes containing a nucleotide sequence of the invention can be obtained, which are capable of high expression of the polynucleotide.

Methods of selecting for transformed, transgenic plants, plant cells or plant tissue culture are routine in the art and can be employed in the methods of the invention provided herein. For example, a recombinant vector of the invention also can include an expression cassette comprising a nucleotide sequence for a selectable marker, which can be used to select a transformed plant, plant part or plant cell. As used herein, “selectable marker” means a nucleotide sequence that when expressed imparts a distinct phenotype to the plant, plant part or plant cell expressing the marker and thus allows such transformed plants, plant parts or plant cells to be distinguished from those that do not have the marker. Such a nucleotide sequence may encode either a selectable or screenable marker, depending on whether the marker confers a trait that can be selected for by chemical means, such as by using a selective agent (e.g., an antibiotic, herbicide, or the like), or on whether the marker is simply a trait that one can identify through observation or testing, such as by screening (e.g., the R-locus trait). Of course, many examples of suitable selectable markers are known in the art and can be used in the expression cassettes described herein.

Examples of selectable markers include, but are not limited to, a nucleotide sequence encoding neo or nptII, which confers resistance to kanamycin, G418, and the like (Potrykus et al. (1985) Mol. Gen. Genet. 199:183-188); a nucleotide sequence encoding bar, which confers resistance to phosphinothricin; a nucleotide sequence encoding an altered 5-enolpyruvylshikimate-3-phosphate (EPSP) synthase, which confers resistance to glyphosate (Hinchee et al. (1988) Biotech. 6:915-922); a nucleotide sequence encoding a nitrilase such as bxn from Klebsiella ozaende that confers resistance to bromoxynil (Stalker et al. (1988) Science 242:419-423); a nucleotide sequence encoding an altered acetolactate synthase (ALS) that confers resistance to imidazolinone, sulfonylurea or other ALS-inhibiting chemicals (EP Patent Application No. 154204); a nucleotide sequence encoding a methotrexate-resistant dihydrofolate reductase (DHFR) (Thillet et al. (1988) J. Biol. Chem. 263:12500-12508); a nucleotide sequence encoding a dalapon dehalogenase that confers resistance to dalapon; a nucleotide sequence encoding a mannose-6-phosphate isomerase (also referred to as phosphomannose isomerase (PMI)) that confers an ability to metabolize mannose (U.S. Pat. Nos. 5,767,378 and 5,994,629); a nucleotide sequence encoding an altered anthranilate synthase that confers resistance to 5-methyl tryptophan; or a nucleotide sequence encoding hph that confers resistance to hygromycin. One of skill in the art is capable of choosing a suitable selectable marker for use in an expression cassette of this invention.

Additional selectable markers include, but are not limited to, a nucleotide sequence encoding β-glucuronidase or uidA (GUS) that encodes an enzyme for which various chromogenic substrates are known; an R-locus nucleotide sequence that encodes a product that regulates the production of anthocyanin pigments (red color) in plant tissues (Dellaporta et al., “Molecular cloning of the maize R-nj allele by transposon-tagging with Ac” 263-282 In: Chromosome Structure and Function: Impact of New Concepts, 18th Stadler Genetics Symposium (Gustafson & Appels eds., Plenum Press 1988)); a nucleotide sequence encoding β-lactamase, an enzyme for which various chromogenic substrates are known (e.g., PADAC, a chromogenic cephalosporin) (Sutcliffe (1978) Proc. Natl. Acad. Sci. USA 75:3737-3741); a nucleotide sequence encoding xylE that encodes a catechol dioxygenase (Zukowsky et al. (1983) Proc. Natl. Acad. Sci. USA 80:1101-1105); a nucleotide sequence encoding tyrosinase, an enzyme capable of oxidizing tyrosine to DOPA and dopaquinone, which in turn condenses to form melanin (Katz et al. (1983) J. Gen. Microbiol. 129:2703-2714); a nucleotide sequence encoding β-galactosidase, an enzyme for which there are chromogenic substrates; a nucleotide sequence encoding luciferase (lux) that allows for bioluminescence detection (Ow et al. (1986) Science 234:856-859); a nucleotide sequence encoding aequorin which may be employed in calcium-sensitive bioluminescence detection (Prasher et al. (1985) Biochem. Biophys. Res. Comm. 126:1259-1268); or a nucleotide sequence encoding green fluorescent protein (Niedz et al. (1995) Plant Cell Reports 14:403-406). One of skill in the art is capable of choosing a suitable selectable marker for use in an expression cassette of this invention.

Further, as is known in the art, intact transgenic plants can be regenerated from transformed plant cells, plant tissue culture or cultured protoplasts using any of a variety of known techniques. Plant regeneration from plant cells, plant tissue culture or cultured protoplasts is described, for example, in Evans et al. (Handbook of Plant Cell Cultures, Vol. 1, MacMilan Publishing Co. New York (1983)); and Vasil I. R. (ed.) (Cell Culture and Somatic Cell Genetics of Plants, Acad. Press, Orlando, Vol. I (1984), and Vol. II (1986)).

Additionally, the genetic properties engineered into the transgenic plants, plant parts, plant cells or seeds of the invention described above can be passed on by sexual reproduction or vegetative growth and therefore can be maintained and propagated in progeny plants. Generally, maintenance and propagation make use of known agricultural methods developed to fit specific purposes such as harvesting, sowing or tilling.

A polynucleotide therefore can be introduced into a plant, plant part or plant cell in any number of ways that are known in the art, as described above. Therefore, no particular method for introducing one or more polynucleotides into a plant is relied upon, rather any method that allows the one or more polynucleotides to be transiently expressed in a plant cell or stably integrated into the genome of the plant can be used. Where more than one polynucleotide is to be introduced, the respective polynucleotides can be assembled as part of a single nucleic acid molecule, or as separate nucleic acid molecules, and can be located on the same or different nucleic acid molecules. Accordingly, the polynucleotides can be introduced into the cell of interest in a single transformation event, in separate transformation events, or, for example, in plants, as part of a breeding protocol.

Additional embodiments of the invention include harvested products produced from the transgenic plants or parts thereof of the invention, as well as a processed product produced from the harvested products. A harvested product can be a whole plant or any plant part, as described herein. Thus, in some embodiments, non-limiting examples of a harvested product include a seed, a fruit, a flower or part thereof (e.g., an anther, a stigma, and the like), a leaf, a stem, and the like. In other embodiments, a processed product includes, but is not limited to, a flour, meal, oil, starch, cereal, and the like produced from a harvested seed or other plant part of the invention, wherein said seed or other plant part comprises a nucleic acid molecule/polynucleotide/nucleotide sequence of this invention.

In other embodiments, the invention provides an extract from a transgenic seed or a transgenic plant of the invention, wherein the extract comprises a nucleic acid molecule, a polynucleotide, a nucleotide sequence or an insecticidal protein of the invention. Extracts from plants or plant parts can be made according to procedures well known in the art (See, de la Torre et al., Food, Agric. Environ. 2(1):84-89 (2004); Guidet, Nucleic Acids Res. 22(9): 1772-1773 (1994); Lipton et al., Food Agric. Immun. 12:153-164 (2000)).

### Assessing Promoter Activity

Several methods are available to assess promoter activity. Expression cassettes are constructed with a visible marker, as described above. Transient transformation methods may be used to assess promoter activity. Using transformation methods such as microprojectile bombardment, Agrobacterium transformation or protoplast transformation, expression cassettes are delivered to plant cells or tissues. Reporter gene activity, such as-glucuronidase activity, luciferase activity or GFP fluorescence is monitored after transformation over time, for example 2 hours, 5 hours, 8 hours, 16 hours, 24 hours, 36 hours, 48 hours and 72 hours after DNA delivery using methods known in the art. Reporter gene activity may be monitored by enzymatic activity, by staining cells or tissue with substrate for the enzyme encoded by the reporter gene or by direct visualization under an appropriate wavelength of light. An insecticidal protein, for example Cry1Ab or Vip3 of the invention, may act as a visible marker whereby the transformed plant cells are tested for insecticidal activity. Full-length promoter sequences, deletions and mutations of the promoter sequence may be assayed and their expression levels compared. Additionally, RNA levels may be measured using methods well known in the art such as Northern blotting, competitive reverse transcriptase PCR and RNAse protection assays. These assays measure the level of expression of a promoter by measuring the ‘steady state’ concentration of a standard transcribed reporter mRNA. This measurement is indirect since the concentration of the reporter mRNA is dependent not only on its synthesis rate, but also on the rate with which the mRNA is degraded. Therefore the steady state level is the product of synthesis rates and degradation rates. The rate of degradation can however be considered to proceed at a fixed rate when the transcribed sequences are identical, and thus this value can serve as a measure of synthesis rates.

Further confirmation of promoter activity is obtained by stable transformation of the promoter in an expression cassette comprising a visible marker or gene of interest into a plant as described above. Using the various methods described above such as enzymatic activity assays, RNA analysis and protein assays as described supra, promoter activity is monitored over development, and additionally by monitoring expression in different tissues in the primary transformants and through subsequent generations of transgenic plants.

### EXAMPLES

The invention will be further described by reference to the following detailed examples. These examples are provided for purposes of illustration only, and are not intended to be limiting unless otherwise specified. Standard recombinant DNA and molecular cloning techniques used here are well known in the art and are described by Ausubel (ed.), Current Protocols in Molecular Biology, John Wiley and Sons, Inc. (1994); J. Sambrook, et al., Molecular Cloning: A Laboratory Manual, 3d Ed., Cold Spring Harbor, NY: Cold Spring Harbor Laboratory Press (2001); and by T. J. Silhavy, M. L. Berman, and L. W. Enquist, Experiments with Gene Fusions, Cold Spring Harbor Laboratory, Cold Spring Harbor, NY (1984).

### Example 1. Identification of Maize Regulatory Elements

Regulatory elements were identified from maize genes on a Syngenta proprietary (Zm80K) Affymetrix chip that are highly expressed in multiple tissue types but not in pollen, i.e. pollen expression was very low or was not detectable. Regulatory elements from eight different maize genes were used to make constructs to drive expression of insect control genes in transgenic corn tissues and plants. A description of the regulatory elements used are shown in Table 1.

TABLE 1
Description of regulatory elements used to make expression constructs.

| Regulatory Element | SEQ ID NO: | Description |
|---|---|---|
| PMP370-3 | 1 | Promoter from the Zm032370 gene (Syngenta chip |
| | | ID), identified as profilin-like. Includes the 5′ UTR. |
| | | the first exon and intron, and a portion of the second |
| | | exon. |
| PMP393-1 | 2 | Promoter from the Zm061393 gene (Syngenta chip |
| | | ID), identified as sucrose synthase. Includes the 5′ |
| | | UTR, the first and second exons, and the first and |
| | | second introns. Also includes a T to A mutation to |
| | | remove the start codon. |
| PMP393-2 | 3 | prZm061393 with a TMV enhancer added at the 3′ |
| | | end. |
| PMP393-3 | 4 | prZm061393-02 with a T to A mutation at bp 8 to |
| | | remove an open reading frame (ORF) |
| PMP393-4 | 5 | prZm041393-01 with a T to A mutation at bp 29 to |
| | | remove an unintended cross component ORF on the |
| | | antisense strand. |
| PMP855-1 | 6 | Promoter from the ZmU45855-3 gene (Syngenta |
| | | chip ID) from Zea mays. Includes the 5′ UTR, the |
| | | first exon, the first intron, and a portion of the |
| | | second exon. |
| PMP747-1 | 7 | Promoter from the Zm001747 gene (Syngenta chip |
| | | ID) from Zea mays, identified as encoding a heat |
| | | shock protein (HSP). Includes the 5′ UTR. |
| PMP004-1 | 8 | Promoter from the Zm021004 gene (Syngenta chip |
| | | ID) from Zea mays, having similarity to genes |
| | | encoding ran-binding proteins, and includes the 5′- |
| | | untranslated region, the first exon, the first intron, |
| | | part of the second exon, and it terminates in a maize |
| | | optimized Kozak sequence. |
| PMP335-1 | 9 | Promoter from the Zm015335 gene (Syngenta chip |
| | | ID) from Zea mays similar to genes encoding 40S |
| | | ribosomal proteins, and includes the 5′-untranslated |
| | | region, the first exon, the first intron, partial second |
| | | exon, and it terminates in a maize optimized Kozak |
| | | sequence. |
| PMP722-1 | 10 | Promoter from the Zm009722 gene from Zea mays, |
| | | and includes the 5′-untranslated region, the first |
| | | exon, the first intron and part of the second exon, |
| | | and it terminates in a maize optimized Kozak |
| | | sequence. |
| PMP948-2 | 11 | Promoter from the Zm058948 (Syngenta chip ID) |
| | | gene from Zea mays. Includes the 5′-untranslated |
| | | region, first exon, first intron, partial second exon, |
| | | and terminates in a maize optimized Kozak |
| | | sequence. |

Terminator elements were also identified from the same genes from which the above-identified promoters were cloned. A description of the terminators is in Table 2.

TABLE 2
Description of terminator elements of the invention.

| Terminator Element | SEQ ID NO: | Description |
|---|---|---|
| t370-2 | 12 | 3′ regulatory sequence from the AF032370 gene |
| | | (Syngenta chip ID) from Zea mays. Includes the 3′ |
| | | UTR and downstream non-transcribed region. |
| t393-1 | 13 | 3′ regulatory sequence from the Zm061393 gene |
| | | (Syngenta chip ID) from Zea mays. Includes the 3′ |
| | | UTR and downstream non-transcribed region. |
| t393-2 | 14 | tZm061393-01 with two point mutations to remove |
| | | unintended ORFS. |
| t855-1 | 15 | 3′ regulatory sequence from the ZmU45855-3 gene |
| | | (Syngenta chip ID) from Zea mays. Includes the 3′ |
| | | UTR and downstream non-transcribed region. |
| t747-1 | 16 | 3′ regulatory sequence from the Zm001747 gene |
| | | (Syngenta chip ID) from Zea mays. Includes the 3′ |
| | | UTR and downstream non-transcribed region. |
| t004-1 | 17 | 3′ regulatory sequence from the Zm021004 gene |
| | | (Syngenta chip ID) from Zea mays. Includes the 3′- |
| | | UTR and downstream non-transcribed region. |
| t335-1 | 18 | 3′ regulatory sequence from the Zm015335 gene |
| | | (Syngenta chip ID) from Zea mays. Includes the 3′ |
| | | UTR and downstream non-transcribed region. |
| t722-1 | 19 | 3′ regulatory sequence from the Zm009722 gene |
| | | (Syngenta chip ID) from Zea mays. Includes the 3′- |
| | | UTR and downstream non-transcribed region. |
| t948-2 | 20 | 3′ regulatory sequence from the Zm058948 gene |
| | | (Syngenta chip ID) from Zea mays. Includes the |
| | | second intron, third exon, 3′-UTR. and downstream |
| | | non-transcribed region. |

### Example 2: Construction of Pollen-Minus Expression Vectors

To test the above-described regulatory elements (promoters and terminators) in transgenic maize plants, eight binary vectors were made. Each binary vector contains two expression cassettes.

The first expression cassette comprises a eFMV/e35S transcriptional enhancer, which is operably linked a promoter of the invention to be tested, which is operably linked to a heterologous coding sequence (cry1Ab) that encodes a Cry1Ab insecticidal protein (U.S. Pat. No. 5,625,136), which is operably linked to the promoter's matching terminator sequence. For example, the expression cassette made to test the prZm061393-01 promoter (SEQ ID NO:2) comprises the tZm061393-01 terminator (SEQ ID NO:13).

The second expression cassette, which was the same for each of the binary vectors, comprises a maize ubiquitin promoter (prUbi1-10) (Christensen et al, 1992 PMB 18: 675) operably linked to a phosphomannose isomerase (PMI; U.S. Pat. No. 5,767,378) coding sequence which is operably linked to a Ubil terminator (tUbi1-01). Expression of PMI allows for positive selection of transgenic plants on mannose.

Both expression cassettes were cloned into a suitable vector for Agrobacterium-mediated maize transformation. The binary vectors that were constructed are listed and described in Table 3.

TABLE 3
Binary vectors constructed to test pollen-minus promoters in maize.

| Binary Vector | SEQ ID NO: | Description |
|---|---|---|
| pSYN18499 | 21 | Binary vector with PMI selection containing cCry1Ab under |
| | | the control of PMP370-3 (PMP370-3:cCry1Ab:t370-2) |
| pSYN18500 | 22 | Binary vector with PMI selection containing cCry1Ab under |
| | | the control of PMP393-1 and t393-1. |
| pSYN18501 | 23 | Binary vector with PMI selection containing cCry1Ab under |
| | | the control of PMP855-1 and t855-1. |
| pSYN18498 | 24 | Binary vector with PMI selection containing cCry1Ab under |
| | | the control PMP747-1. (PMP747:cCry1Ab:t747) |
| pSYN18617 | 25 | Binary vector with PMI selection containing cCry1Ab under |
| | | the control of PMP004-1. (PMP004-1:cCry1Ab:t004-1). |
| pSYN18618 | 26 | Binary vector with PMI selection containing cCry1Ab under |
| | | the control of PMP335-1. (prZm015335-01:cCry1Ab:t335-1) |
| pSYN18619 | 27 | Binary vector with PMI selection containing cCry1Ab under |
| | | the control of PMP722-1 (PMP722-1:cCry1Ab:t722-1). |
| pSYN18705 | 28 | Binary vector with PMI selection containing cCry1Ab under |
| | | the control of PMP948-2 (PMP948-2:cCry1Ab:t948-2). |

### Example 3: Transient Expression of Cry1Ab in Maize Directed by Pollen-Minus Promoters

The vectors described above were transferred into Agrobacterium tumefactions strain LBA4404 containing helper plasmid (pSBI) using a freeze-thaw method (An et al., Binary vector. In: Gelvin S B, Schilproot R A (eds), Plant molecular biology manual. Kluwar Academic Publishers, Dordrecht, pp A3 1-19 (1988)). Preparation of Agrobacterium cultures was carried out as described by Azhakanandam et al., Plant Mol. Biol. 63: 393-404 (2007). In brief, the genetically modified agrobacteria were grown overnight in 50 mL of YP medium containing 100 □M acetosyringone and 10 □M MES (pH 5.6), and subsequently were pelleted by centrifugation at 4000×g for 10 min. The pellets were resuspended in the infection medium (Murashige and Skoog salts with vitamins, 2% sucrose, 500 □M MES (pH 5.6), 10 □M MgSO4, and 100 □M acetosyringone) to OD600=0.5 and subsequently held at 28° C. for 2-3 hours.

A maize seedling in planta transient expression assay was performed essentially as described in U.S. Pat. No. 8,642,839, incorporated herein by reference in its entirety. Briefly, maize seeds were germinated under greenhouse conditions in 2.5 inch pots filled with Fafard germination mix. Seedlings were kept under a 14/10 day/night cycle with a day light intensity of 2000 □-mol-m-2s-1 maintained with supplemental lighting. The temperature was maintained between 23° C.-26° C. The agroinfiltration experiment performed mostly using primary and secondary leaves of V2 stage (Ritchie S. W., Hanway J. J. Benson G. O. (edts): How a Corn Plant Develops: Iowa State Univ Special Report No. 48, July 2005). To make infiltration easier, the seedlings were watered 1-2 hours prior to agroinfiltration, which keeps the leaf turgid and stomata open. Infiltration of individual leaves was carried out on maize seedlings using a 5 ml syringe body (BD 5 ml syringe with Luer-Lok.TM. Tip, BD.TM. Franklin Lakes, N.J. 07427, USA), by pressing the tip of the syringe against the abaxial surface of the leaf. The first and second visible leaves of V2 stage were infiltrated with: 1 ml of agrobacterium suspension/28 seconds/leaf. Infiltrated plants were transferred and maintained under growth chamber conditions set at 25° C. with a 16/8 day/night cycle with a light intensity of 1900 □-mol-m-2s-1. Plant tissue was harvested after 4 days post infiltration for subsequent analysis of Cry1Ab protein and PMI protein detection using ELISA. Results demonstrated that both proteins were detected in all eight vector groups, indicating that the promoters of the invention are capable of driving transgene expression in maize cells.

### Example 4: Expression of Cry1Ab in Stably Transformed Corn Using Pollen-Minus Promoters

Agrobacterium transformation of immature maize embryos was performed essentially as described in Negrotto et al., 2000, Plant Cell Reports 19: 798-803, herein incorporated by reference in its entirety. However, various media constituents known in the art may be substituted. Briefly, Agrobacterium strain LBA4404 (pSB1) containing a plant transformation vector described above was grown on YEP (yeast extract (5 g/L), peptone (10 g/L), NaCl (5 g/L), 15 g/l agar, pH 6.8) solid medium for about 2-4 days at about 28° C. Approximately 0.8×10⁹ Agrobacterium were suspended in LS-inf media supplemented with 100 □M As (Negrotto et al., supra). Bacteria were pre-induced in this medium for about 30-60 minutes.

Immature embryos from a suitable genotype were excised from about 8-12 day old ears into liquid LS-inf+100 □M As. Embryos were rinsed once with fresh infection medium. Agrobacterium solution was then added and embryos were vortexed for about 30 seconds and allowed to settle with the bacteria for about 5 minutes. The embryos were then transferred scutellum side up to LSAs medium and cultured in the dark for two to three days. Subsequently, between 20 and 25 embryos per petri plate were transferred to LSDc medium supplemented with cefotaxime (250 mg/l) and silver nitrate (1.6 mg/l) and cultured in the dark for 28° C. for 10 days.

Immature embryos, producing embryogenic callus were transferred to LSD1M0.5S medium. The cultures were selected on this medium for about 6 weeks with a subculture step at about 3 weeks. Surviving calli were transferred to Reg1 medium supplemented with mannose. Following culturing in the light (16 hour light/8 hour dark regiment), green tissues were then transferred to Reg2 medium without growth regulators and incubated for about 1-2 weeks. Plantlets were transferred to Magenta GA-7 boxes (Magenta Corp, Chicago Ill.) containing Reg3 medium and grown in the light. After about 2-3 weeks, plants were tested for the presence of the pmi gene and the cry1Ab-09 coding sequence. Positive plants from the PCR assay are transferred to the greenhouse and tested for expression level of Cry1Ab-09 protein in leaves and pollen and tested for activity against European corn borer (Ostrinia nubilalis).

The concentrations of the Cry1Ab protein were determined at the vegetative (V) and reproductive (R) stages of growth of the transgenic maize plants. The “V” stages are designated numerically as V1, V2, V3, etc. by the number of leaves through the VT stage where the last branch of the tassel is visible. The “R” stages are designated R1 to R6 from silking to physiological maturity of the ear, respectively. Cry1Ab was quantified by ELISA in V3-V4 and VT stage leaves as well as silk, husk and pollen from 8-12 events for each promoter tested using ELISA protocols known in the art. Briefly, leaf tissue is lyophilized and then reduced to a fine powder by processing using either a coffee grinder, blender, Grindomix™ grinder (Brinkmann Instruments; Westbury, N.Y., USA), mortar with a pestle or mill, or a combination of these devices. All processing is done in the presence of either dry ice or liquid nitrogen. Samples are mixed well to ensure homogeneity. The percent dry weight of each sample is determined and the processed samples are stored at about −80° C. until lyophilization.

For each sample analyzed, a 1.0 g aliquot of the powdered plant material (other than pollen) is weighed into a 15-ml polypropylene tube, suspended in 3 ml extraction buffer [50 mM CAPS, 0.1 M NaCl, 2 mM EDTA, 1 mM dithiothreitol, 1 mM 4-(1-aminoethyl) benzenesulfonyl fluoride HCl, 1 mM leupeptin, pH 10], and extracted using an Autogizer® homogenizer (Tomtek; Hamden, Conn., USA). After centrifugation for 15 min at 10,000×g at 4° C., the supernatant is used for Cry1Ab and PMI analysis by ELISA. After treatment with iodoacetamide as described by Hill and Straka (1988), total protein in the extracts is quantitated using the BCA™ Protein Assay Reagent (Pierce; Rockford, Ill., USA).

Maize pollen extracts are prepared by suspending pollen 1:30 (w/v) in extraction buffer. After about 30 min on ice, the pollen suspensions are disrupted by three passages through a French pressure cell at about 15,000 psi, followed by centrifugation at 14,000×g for about 5 min at 4° C. Cry1Ab and PMI analyses by ELISA were performed on the supernatants as described below. Total protein was quantitated as described above.

The extracts prepared as described above were quantitatively analyzed for Cry1Ab and PMI by ELISA (Tijssen, 1985). Cry1Ab was quantified using immuno-affinity purified monoclonal, anti-Cry1Ab antibody and immuno-affinity purified polyclonal anti-Cry1Ab antibody. The PMI was quantified using Protein A-purified polyclonal rabbit and immunoaffinity-purified polyclonal goat antibodies specific for PMI. The lower limit of quantification of the double-sandwich ELISA for Cry1Ab and PMI was estimated based on the lowest concentration of pure reference protein lying on the linear portion of the standard curve, the maximum volume of a control extract that could be analyzed without background interference, and the corresponding weight of the sample that the aliquot represented.

The Cry1Ab ELISA results are shown in Table 4. The level of detection (LOD) for the Cry1Ab ELISA test was determined to be about 0.37 ng/mg total soluble protein (TSP). Quantifiable levels of Cry1Ab protein were detected in V3-V4 leaves, silks, husks and VT leaves from all events for each of the promoters tested, which were significantly higher than in pollen. Concentrations of Cry1Ab in pollen for each of the promoters was only slightly above the LOD for the ELISA. Five out of the eight promoters tested expressed levels of Cry1Ab in non-pollen tissues that were greater than 1000-fold higher than the LOD. Three of the eight promoters expressed Cry1Ab at levels that were about 500- to about 950-fold higher than the LOD. By comparison, seven of the eight promoters tested expressed Cry1Ab in pollen at levels that were less than 10-fold higher than the LOD, and one promoter, prZmU45855, expressed Cry1Ab in pollen at a concentration that was 15-fold higher than the LOD. However, some plants from each event of each of the vector groups comprising the 001747-01, 061393-01, AF032370-02, 009722-01, 015335-01 or the 058948-02 promoter had no detectable levels of Cry1Ab in pollen. Cry1Ab was expressed at insecticidal levels in all non-pollen tissues tested, whereas Cry1Ab levels in pollen for all of the promoters tested were below the concentration necessary for insecticidal activity.

TABLE 4
Cry1Ab Expression in Tissues of Transgenic Maize Plants.

| | | Mean Cry1Ab Level | | | | |
|---|---|---|---|---|---|---|
| | | (ng/mg TSP) | | | | |
| | | V3-V4 | | | | |
| Vector | Promoter | Leaf | Silk | Husk | VT Leaf | Pollen |
| pSYN18499 | PMP370-2 | 389 | 485 | 290 | 229 | 1.6 |
| pSYN18500 | PMP393-1 | 927 | 359 | 437 | 402 | 2.2 |
| pSYN18501 | PMP855-1 | 910 | 461 | 238 | 693 | 5.7 |
| pSYN18498 | PMP747-1 | 324 | 491 | 505 | 341 | 1.1 |
| pSYN18617 | PMP004-1 | 1037 | 194 | 960 | 462 | 2.7 |
| pSYN18618 | PMP335-1 | 254 | 109 | 243 | 256 | 0.3 |
| pSYN18619 | PMP722-1 | 1132 | 283 | 390 | 476 | 1.3 |
| pSYN18705 | PMP948-2 | 292 | 215 | 409 | 318 | 1.7 |

### Example 5. Expression of a Cry1Ai in Stably-Transformed Maize

Maize plants were transformed as described above with an expression cassette comprising a eFMV/e35S transcriptional enhancer operatively linked to the PMP393-4 promoter of the invention (SEQ ID NO:5), operatively linked to a heterologous coding sequence that encodes a full-length Cry1Ai insecticidal protein (US Application publication No. 20190177377), which is operatively linked to the t393-2 terminator of the invention (SEQ ID NO:14). V3-V4 leaf tissue and pollen from 40 events were assayed as described above. The level of detection (LOD) for the Cry1Ai ELISA test was determined to be about 0.75 ng/mg total soluble protein (TSP). Results of the ELISA on the tissues from the 40 transgenic maize events demonstrated that the level of Cry1Ai protein in the V3-V4 leaves ranged from about 10 ng/mg TSP to about 80 ng/mg TSP, whereas no Cri1Ai protein was not detected in pollen, indicating that the level of expression in pollen was below the LOD of the ELISA, i.e. <0.75 ng/mg TSP, in all 40 events.

All publications and patent applications mentioned in this specification are indicative of the level of skill of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious to persons skilled in the art that certain changes and modifications may be practiced within the scope of the present invention.

1. An expression cassette comprising a promoter comprising the nucleotide sequence of SEQ ID NO:6 or SEQ ID NO:10 operably linked to a heterologous polynucleotide of interest, which is operably linked to a 3′-untranslated region including a polyadenylation signal.

2. The expression cassette of claim 1, wherein the heterologous polynucleotide encodes an insecticidal protein or a double stranded RNA (dsRNA).

3-13. (canceled)

14. A recombinant vector comprising the expression cassette of claim 1.

15. A plant cell comprising the expression cassette of claims 1.

16-18. (canceled)

19. A transgenic plant comprising the expression cassette of claim 1.

20. (canceled)

21. The transgenic plant of claim 19, wherein said plant is a monocot.

22. The transgenic plant of claim 21, wherein the monocot is maize.

23-24. (canceled)

25. A transgenic seed of the plant of claim 19, wherein the seed comprises the expression cassette.

26. The transgenic plant of claim 19, wherein the heterologous polynucleotide encodes an insecticidal protein.

27-33. (canceled)

34. A method for expressing a protein or polynucleotide of interest in a plant or a plant cell comprising introducing into the plant or the plant cell an expression cassette comprising a promoter operably linked to a heterologous polynucleotide that encodes the protein or polynucleotide of interest, wherein said promoter comprises the nucleotide sequence of SEQ ID NO:6 or SEQ ID NO:10.

35. The method of claim 34, wherein the protein of interest is an insecticidal protein or the polynucleotide of interest is a dsRNA that is insecticidal.

36-42. (canceled)

43. The method of claim 34, wherein the plant or plant cell is a maize plant or plant cell.

44. (canceled)

45. An isolated nucleic acid molecule comprising the nucleotide sequence of SEQ ID NO:6 or SEQ ID NO:10.

46-64. (canceled)

65. (canceled)

66. The expression cassette of claim 1, wherein the 3′-untranslated region comprises a terminator sequence comprising the sequence of SEQ ID NO: 12, 13, 14, 15, 16, 17, 18, 19, and 20.

67. The recombinant vector of claim 14, wherein said vector is a binary vector comprising SEQ ID NO:23 or SEQ ID NO:27.

68. A method of specifically expressing a heterologous coding sequence in a transgenic plant tissue that is not pollen, comprising: a) transforming at least one plant cell with a vector, wherein said vector comprises the expression cassette of claim 1; b) growing the transgenic plant cell comprising said expression cassette; and c) producing at least one transgenic plant from said transformed plant cell wherein the heterologous coding sequence is specifically expressed in a tissue that is not pollen under control of said nucleotide sequence.


SEQUENCE LISTING (29 sequences)

SEQ ID NO: 1 - DNA, 2738 bp, Zea mays
  source 1..2738: mol_type = genomic DNA
gagatatgtgatgtatatgtgatatatgtgatgattatgtgatatattttttgtttgtttggatggaataataaaaacaaataaaaagggtatgttggccacttttggcgagtgtaacactcagcaaagaggtactttgccgagtgtcacagtcatatcattcggcaaagaaggcacacatgggaaccgataaagcttctttgccgagtgttgtggccttagcactcggcaaagaagcaacttttgccgagagcctcctagtgtactcggcaaaggaactgacaaaggagcccactgatgattcctttgatgagtggtagtccggcagacacacggcaaagataaagtctttgccgagtgccacctaatacgcccggcaaaggaactggcaaaggacacacgatgagtttatttgtcgagtgctagtacaatagacactcgacaaagagtgagcctttgcagagtgtcaccgtggcactcggcaaagtccactctttgtcgagtgtcaccgtgacactcagcgaagtctccgtcgctgtcatctgtcgccgtgacgtcgacttttttttgccgagtaccgagtggttgccgagtgtccgacaaaaaatactcggttaaagaccgttgccgatgtcagttcaccaagaccttttttatcaagtgtcatacttgataaagtcttccccaagtattttcaaaactttgtcgattacctcacacaccaaggggtaggatcgtgttgaggcggtttgctgcttgtttcttgctttcgccgaaccaacggaccatgagcgcataaaccaatgggcgagcggccgcatatacaggcctgcgcggcgagcctcaccttgctggatttgaatgccccctcggcatggtcaatgaccatgcatctttgtgcagaacatataaaatgcacaaattaattaattaataaaaatacatgaaagagtacataatttgtttgtggattattaattatatttataaatatattatagtatattatattttatctttatctaactacaaaaataattatagattgttaacttttttttgcattactggtaagattttgtcgattttataattttgtctaacatgttttattatcaatctaaaattgcaaattatatgttaacgaactaaatttgtaataaaatatatgtaataaaggtcgtctaaaagtaagctaaaattcacgcacctaaattttatttagtaaagtgttttttcttgacatatgacactagtgttttcaaagcttgtctcttaatttaggattaaattaataggcctctctaaattgctttttataagataaataaatcctattccgtctgctcatcttcaaagaacatttcaatgaaacaaatatttgggaaaaccaatggaaaaagaataaaaataaaaattgaaaaagagaagtggaggaaaagtagggttccattccacggcctgtcgagtgtcgacccacaaaccacgcaaccgatatattccccagcacccccagtctccagccgtccaaccgagacaccgcgtgcgaaccaagcagaccacaacaagaagaagcgtagtcgtcgccggaaggaaaggcgcggagcaagatctcgtggcaggcgtacgtcgacgagcacctgaagtgcgagatcgaaggacagcatctcagcgccgccgccatcgtcggtcacgacggcagcgtttgggcgcagtccgagagcttccccgaggtcattcactcccaccttatctcttcccgctctacttgcttagttgctttatccatgttcagtgctcctgcgctgatttagacccgcgcgactctggatctgccccagccgtgcggcccggccggatcggagcgaggggatgtccggatctcggtcgctgaggcgagatgcggatctaaggccccttccctcggtgacgtggttgctgctgctgctgttctccgggcgtttgatgccaatatagctgagatcaagcttgatgatctgcgtactgtggatttgctagtgagggatggccggatcgggctgttggcgtccgacctgatttggttgttcgcttgaatagacttacttaccagtggacagtggtagtgatcgttcagcagcagtagagcaatttgttttacatgtaaaatttgagtggctgctagagatgcaccaatggccgagctgcatgtgtttattcctatatagcgacagttgtgtgattgatccaaaacgcaaaattttagttttcatcaaatgatgatttcctgatgccactacctgtgtgtgaggcacttattaaatgaaaagtaactttcggttgggatctgggaatctgtattgtgttccgtggttggtcacagcttgtggcctgcattgttccataggccaaatggctaggcaaggaaaataaatcatgtaatttggaaaaaaataactgccatagtcagtagtgtacagcagtacatgcttgtccgggccttttatttcaccatcgtgttgtggtatatggagtacttggattgtgcgatgctttatacatttgtttgttctagcagagtttgagtttatatttctattcttatgtagggcgtcacgagtgtcgtcaattgttacatccttacatccttgtgttcacaatatgatccatcaactctttcttctgcggtctcttatccctgtgttctcatgtccatgtttcctgcttcttgttgcagttaaagccttaggaggtaaacc

SEQ ID NO: 2 - DNA, 2627 bp, synthetic construct
  misc_feature 1..2627: note = The 5' regulatory sequence from the Zm061393 gene with a T to A mutation to remove a start codon.
  source 1..2627: mol_type = other DNA
tctgcctttctgttcttcaaacgatgtctcatgtctgcgctggacaactttcttgttgccgcctgtcgcttgcgctgtgctgactggacgcagctccggaggtttggttgtgcttggttttcgtagagaactcgccacttgccgcccgcacgttcttggtgtttcctcctccccgctgtgttctgcgcacgggctttttctgagagacccatgtttcccttttacttttataaacagtatacatgctatgtttctagaaggaggggaaacctaatccccctaatccaatggcggggaggaaatagggtggggtggggtggggggagggaaatatctcgctactttttaatccggacaagctcatttgcgtttgcgtctgaatgatgatgactgcaatgctgatcgcacctcgggtgtcggatcaccagcttttggctgctctcaccaaatcagctgcaagaagattagagcacaaaagaattacagaaagagagcctttttcttttcttccttgtggggttcctttcatttcgtgctctcctttctctgccagccagtccgtccttgcgtccactgcacctgcacacaggtcaccccgacccgcactgttctagactccattagaaaaaaaaaggtctgaacctttccgaaaccagccagccattggtctggcaggccagcatatgctaattggatttttttgccgcatcattgagtgcgccatcaggatttggaaatcctggttttgagtaatacagtaatttggcattatccattgccgaattcccaagctccgtcagcttgaacgtggacccctaccatctgcaccagctcggcacctcacgctcgcagcgctaggagcctaggagcagctgcccgtctatttattggtccctctcccgtcccagagaaaccctccctccctcctccattggactgcttgctccctgttgaccattggggtatgcttgctgccttgctctcctgttcatctccgtgctaaacctctgtcctctgggtgggtttttgctgggattttgagctaatctgctggtcccggtagaaaaagatcatgtcccctgacgtgctcaagcgctcgccttagccgcgtccttgccccccgccattttttgcggtttcggtgtgttcccgtgactcgccgggtgcgtcatcgcctgaatcttgtctgggctctgctgacatgttcttggctagttgggtttatagattcctctgatctaaaccgtgcctgtgctgcgcacagaactctcccctgtcctttcctggggttttggttacgtggtggtagtaagcttggatttgcacatggataaagttgttctaagctccgtgggttgcttgagatcttgctgttattgcgtgccgtgctcactttttttgcaatccgaggaatgaatttgtcgtttactcgttttggtggattattagcgcgaaaaaaaaactcttttttttttgttcttttactacgaaaagcatcttcttggattttgctatcttcttttactacgaaaaactcttgagtctaggaatttgaatttgtgatgtccattcttgcagtgcgctgtgctttattgggaagccaaatcctattattttctgcctctagggtctgaatggaatcagtactcttgagacagaaaatcaatccaatcaagttgatttctttctttaaaaatattatcacagaactaagtgcttgtgcggaatcagtactggcttttgtttggtggaggatcaatacttgcttttgtttgggggtggcaactgttttgctataagattccatgtgttcctgttgagatgaatcatatatagtatagctgcatactacaaatctgtttttcaaatttaggttgctttggcatgatctatttttttgtcagacagactttctaagtggtagctcttgatttcttgttcttgtacaactggtgctgctgaatcttgaccgtatagctcgaattgcagtattctgaaccatcgagccaaggctgccaagctgactcgcctccacagtcttcgcgaacgccttggtgccaccttctcctcccatcccaatgaactgatagcactcttttccaggtgggcttaccaaaatcatataacttgcatttcattcggtactgaaagttgttaatttgttattctcttcatgcctgtcttaatagcacacccagatgtaaacacgagattatgcaacttcttacttggtttcttttgttggcaccatcatgcatgctaattgctaaggatgttacctattcatccttgactcatattatcatatgtaatgattttatgatcacgagactattgattgtgaagcatagtatagctgttcttcagtttttgtacccttttgtttttttccttaagctagaactggtacaatttagttgataagacagtgtagtttgtagtacgtcatttgacagattgtttgtctttagctggtaaagtgccatttaatatctgtatccttcagatctaataaaaaggatatgagatgtccatcacaagaggggaaaaattacatgatctgagatgtaacatccgtttttatttgtgaaataccacttctacaggtatcttcactagggtaaacc

SEQ ID NO: 3 - DNA, 2695 bp, synthetic construct
  misc_feature 1..2695: note = TMV enhancer added to the 3' end.
  source 1..2695: mol_type = other DNA
tctgcctttctgttcttcaaacgatgtctcatgtctgcgctggacaactttcttgttgccgcctgtcgcttgcgctgtgctgactggacgcagctccggaggtttggttgtgcttggttttcgtagagaactcgccacttgccgcccgcacgttcttggtgtttcctcctccccgctgtgttctgcgcacgggctttttctgagagacccaagtttcccttttacttttataaacagtatacatgctatgtttctagaaggaggggaaacctaatccccctaatccaatggcggggaggaaatagggtggggtggggtggggggagggaaatatctcgctactttttaatccggacaagctcatttgcgtttgcgtctgaatgatgatgactgcaatgctgatcgcacctcgggtgtcggatcaccagcttttggctgctctcaccaaatcagctgcaagaagattagagcacaaaagaattacagaaagagagcctttttcttttcttccttgtggggttcctttcatttcgtgctctcctttctctgccagccagtccgtccttgcgtccactgcacctgcacacaggtcaccccgacccgcactgttctagactccattagaaaaaaaaaggtctgaacctttccgaaaccagccagccattggtctggcaggccagcatatgctaattggatttttttgccgcatcattgagtgcgccatcaggatttggaaatcctggttttgagtaatacagtaatttggcattatccattgccgaattcccaagctccgtcagcttgaacgtggacccctaccatctgcaccagctcggcacctcacgctcgcagcgctaggagcctaggagcagctgcccgtctatttattggtccctctcccgtcccagagaaaccctccctccctcctccattggactgcttgctccctgttgaccattggggtatgcttgctgccttgctctcctgttcatctccgtgctaaacctctgtcctctgggtgggtttttgctgggattttgagctaatctgctggtcccggtagaaaaagatcatgtcccctgacgtgctcaagcgctcgccttagccgcgtccttgccccccgccattttttgcggtttcggtgtgttcccgtgactcgccgggtgcgtcatcgcctgaatcttgtctgggctctgctgacatgttcttggctagttgggtttatagattcctctgatctaaaccgtgcctgtgctgcgcacagaactctcccctgtcctttcctggggttttggttacgtggtggtagtaagcttggatttgcacatggataaagttgttctaagctccgtgggttgcttgagatcttgctgttattgcgtgccgtgctcactttttttgcaatccgaggaatgaatttgtcgtttactcgttttggtggattattagcgcgaaaaaaaaactcttttttttttgttcttttactacgaaaagcatcttcttggattttgctatcttcttttactacgaaaaactcttgagtctaggaatttgaatttgtgatgtccattcttgcagtgcgctgtgctttattgggaagccaaatcctattattttctgcctctagggtctgaatggaatcagtactcttgagacagaaaatcaatccaatcaagttgatttctttctttaaaaatattatcacagaactaagtgcttgtgcggaatcagtactggcttttgtttggtggaggatcaatacttgcttttgtttgggggtggcaactgttttgctataagattccatgtgttcctgttgagatgaatcatatatagtatagctgcatactacaaatctgtttttcaaatttaggttgctttggcatgatctatttttttgtcagacagactttctaagtggtagctcttgatttcttgttcttgtacaactggtgctgctgaatcttgaccgtatagctcgaattgcagtattctgaaccatcgagccaaggctgccaagctgactcgcctccacagtcttcgcgaacgccttggtgccaccttctcctcccatcccaatgaactgatagcactcttttccaggtgggcttaccaaaatcatataacttgcatttcattcggtactgaaagttgttaatttgttattctcttcatgcctgtcttaatagcacacccagatgtaaacacgagattatgcaacttcttacttggtttcttttgttggcaccatcatgcatgctaattgctaaggatgttacctattcatccttgactcatattatcatatgtaatgattttatgatcacgagactattgattgtgaagcatagtatagctgttcttcagtttttgtacccttttgtttttttccttaagctagaactggtacaatttagttgataagacagtgtagtttgtagtacgtcatttgacagattgtttgtctttagctggtaaagtgccatttaatatctgtatccttcagatctaataaaaaggatatgagatgtccatcacaagaggggaaaaattacatgatctgagatgtaacatccgtttttatttgtgaaataccacttctacaggtatcttcactagggtttgtcgaacaacaacaaacaacaaacaacaaagtcgaacaacaacaaacaacaaacaacaaagtcgaccaaaa

SEQ ID NO: 4 - DNA, 2694 bp, synthetic construct
  misc_feature 1..2694: note = Includes point mutation to eliminate ORFS.
  source 1..2694: mol_type = other DNA
tctgcctatctgttcttcaaacgatgtctcatgtctgcgctggacaactttcttgttgccgcctgtcgcttgcgctgtgctgactggacgcagctccggaggtttggttgtgcttggttttcgtagagaactcgccacttgccgcccgcacgttcttggtgtttcctcctccccgctgtgttctgcgcacgggctttttctgagagacccaagtttcccttttacttttataaacagtatacatgctatgtttctagaaggaggggaaacctaatccccctaatccaatggcggggaggaaatagggtggggtggggtggggggagggaaatatctcgctactttttaatccggacaagctcatttgcgtttgcgtctgaatgatgatgactgcaatgctgatcgcacctcgggtgtcggatcaccagcttttggctgctctcaccaaatcagctgcaagaagattagagcacaaaagaattacagaaagagagcctttttcttttcttccttgtggggttcctttcatttcgtgctctcctttctctgccagccagtccgtccttgcgtccactgcacctgcacacaggtcaccccgacccgcactgttctagactccattagaaaaaaaaaggtctgaacctttccgaaaccagccagccattggtctggcaggccagcatatgctaattggatttttttgccgcatcattgagtgcgccatcaggatttggaaatcctggttttgagtaatacagtaatttggcattatccattgccgaattcccaagctccgtcagcttgaacgtggacccctaccatctgcaccagctcggcacctcacgctcgcagcgctaggagcctaggagcagctgcccgtctatttattggtccctctcccgtcccagagaaaccctccctccctcctccattggactgcttgctccctgttgaccattggggtatgcttgctgccttgctctcctgttcatctccgtgctaaacctctgtcctctgggtgggtttttgctgggattttgagctaatctgctggtcccggtagaaaaagatcatgtcccctgacgtgctcaagcgctcgccttagccgcgtccttgccccccgccattttttgcggtttcggtgtgttcccgtgactcgccgggtgcgtcatcgcctgaatcttgtctgggctctgctgacatgttcttggctagttgggtttatagattcctctgatctaaaccgtgcctgtgctgcgcacagaactctcccctgtcctttcctggggttttggttacgtggtggtagtaagcttggatttgcacatggataaagttgttctaagctccgtgggttgcttgagatcttgctgttattgcgtgccgtgctcactttttttgcaatccgaggaatgaatttgtcgtttactcgttttggtggattattagcgcgaaaaaaaaactcttttttttttgttcttttactacgaaaagcatcttcttggattttgctatcttcttttactacgaaaaactcttgagtctaggaatttgaatttgtgatgtccattcttgcagtgcgctgtgctttattgggaagccaaatcctattattttctgcctctagggtctgaatggaatcagtactcttgagacagaaaatcaatccaatcaagttgatttctttctttaaaaatattatcacagaactaagtgcttgtgcggaatcagtactggcttttgtttggtggaggatcaatacttgcttttgtttgggggtggcaactgttttgctataagattccatgtgttcctgttgagatgaatcatatatagtatagctgcatactacaaatctgtttttcaaatttaggttgctttggcatgatctatttttttgtcagacagactttctaagtggtagctcttgatttcttgttcttgtacaactggtgctgctgaatcttgaccgtatagctcgaattgcagtattctgaaccatcgagccaaggctgccaagctgactcgcctccacagtcttcgcgaacgccttggtgccaccttctcctcccatcccaatgaactgatagcactcttttccaggtgggcttaccaaaatcatataacttgcatttcattcggtactgaaagttgttaatttgttattctcttcatgcctgtcttaatagcacacccagatgtaaacacgagattatgcaacttcttacttggtttcttttgttggcaccatcatgcatgctaattgctaaggatgttacctattcatccttgactcatattatcatatgtaatgattttatgatcacgagactattgattgtgaagcatagtatagctgttcttcagtttttgtacccttttgtttttttccttaagctagaactggtacaatttagttgataagacagtgtagtttgtagtacgtcatttgacagattgtttgtctttagctggtaaagtgccatttaatatctgtatccttcagatctaataaaaaggatatgagatgtccatcacaagaggggaaaaattacatgatctgagatgtaacatccgtttttatttgtgaaataccacttctacaggtatcttcactagggtttgtcgaacaacaacaaacaacaaacaacaaagtcgaacaacaacaaacaacaaacaacaaagtcgaccaaa

SEQ ID NO: 5 - DNA, 2626 bp, synthetic construct
  misc_feature 1..2626: note = Comprises point mutation to eliminate cross component ORF on antisense strand.
  source 1..2626: mol_type = other DNA
tctgcctttctgttcttcaaacgatgtcacatgtctgcgctggacaactttcttgttgccgcctgtcgcttgcgctgtgctgactggacgcagctccggaggtttggttgtgcttggttttcgtagagaactcgccacttgccgcccgcacgttcttggtgtttcctcctccccgctgtgttctgcgcacgggctttttctgagagacccatgtttcccttttacttttataaacagtatacatgctatgtttctagaaggaggggaaacctaatccccctaatccaatggcggggaggaaatagggtggggtggggtggggggagggaaatatctcgctactttttaatccggacaagctcatttgcgtttgcgtctgaatgatgatgactgcaatgctgatcgcacctcgggtgtcggatcaccagcttttggctgctctcaccaaatcagctgcaagaagattagagcacaaaagaattacagaaagagagcctttttcttttcttccttgtggggttcctttcatttcgtgctctcctttctctgccagccagtccgtccttgcgtccactgcacctgcacacaggtcaccccgacccgcactgttctagactccattagaaaaaaaaaggtctgaacctttccgaaaccagccagccattggtctggcaggccagcatatgctaattggatttttttgccgcatcattgagtgcgccatcaggatttggaaatcctggttttgagtaatacagtaatttggcattatccattgccgaattcccaagctccgtcagcttgaacgtggacccctaccatctgcaccagctcggcacctcacgctcgcagcgctaggagcctaggagcagctgcccgtctatttattggtccctctcccgtcccagagaaaccctccctccctcctccattggactgcttgctccctgttgaccattggggtatgcttgctgccttgctctcctgttcatctccgtgctaaacctctgtcctctgggtgggtttttgctgggattttgagctaatctgctggtcccggtagaaaaagatcatgtcccctgacgtgctcaagcgctcgccttagccgcgtccttgccccccgccattttttgcggtttcggtgtgttcccgtgactcgccgggtgcgtcatcgcctgaatcttgtctgggctctgctgacatgttcttggctagttgggtttatagattcctctgatctaaaccgtgcctgtgctgcgcacagaactctcccctgtcctttcctggggttttggttacgtggtggtagtaagcttggatttgcacatggataaagttgttctaagctccgtgggttgcttgagatcttgctgttattgcgtgccgtgctcactttttttgcaatccgaggaatgaatttgtcgtttactcgttttggtggattattagcgcgaaaaaaaaactcttttttttttgttcttttactacgaaaagcatcttcttggattttgctatcttcttttactacgaaaaactcttgagtctaggaatttgaatttgtgatgtccattcttgcagtgcgctgtgctttattgggaagccaaatcctattattttctgcctctagggtctgaatggaatcagtactcttgagacagaaaatcaatccaatcaagttgatttctttctttaaaaatattatcacagaactaagtgcttgtgcggaatcagtactggcttttgtttggtggaggatcaatacttgcttttgtttgggggtggcaactgttttgctataagattccatgtgttcctgttgagatgaatcatatatagtatagctgcatactacaaatctgtttttcaaatttaggttgctttggcatgatctatttttttgtcagacagactttctaagtggtagctcttgatttcttgttcttgtacaactggtgctgctgaatcttgaccgtatagctcgaattgcagtattctgaaccatcgagccaaggctgccaagctgactcgcctccacagtcttcgcgaacgccttggtgccaccttctcctcccatcccaatgaactgatagcactcttttccaggtgggcttaccaaaatcatataacttgcatttcattcggtactgaaagttgttaatttgttattctcttcatgcctgtcttaatagcacacccagatgtaaacacgagattatgcaacttcttacttggtttcttttgttggcaccatcatgcatgctaattgctaaggatgttacctattcatccttgactcatattatcatatgtaatgattttatgatcacgagactattgattgtgaagcatagtatagctgttcttcagtttttgtacccttttgtttttttccttaagctagaactggtacaatttagttgataagacagtgtagtttgtagtacgtcatttgacagattgtttgtctttagctggtaaagtgccatttaatatctgtatccttcagatctaataaaaaggatatgagatgtccatcacaagaggggaaaaattacatgatctgagatgtaacatccgtttttatttgtgaaataccacttctacaggtatcttcactagggtaaac

SEQ ID NO: 6 - DNA, 2510 bp, Zea mays
  source 1..2510: mol_type = genomic DNA
atgccttacgatcatctgacatcttaaatatttcaaactgcagtatttcaaaaactgtggttttgtcaaaaactttgttcccaaactgagactaaaatgtagtgctagataataaggccttacccgttggaatgacaaaaaagaaacagaaaacacctgaaaaatagtattgccttggctctaaattataaatcgttttggttttatttaatatctattttttaagcatactaaaatctatgtacctatgaaaaataaaatatgaactacaatttaagactgagtacagtgtacagcgataagttatatgaaccacaattgtttttgaaaggaaaaaaactaaattgacaagagaaacaatcaactcttttagaaactttgtataccaaaaaacgaaataaagaagaagaaaaaaatacacacgatgagtttattagcccacaaaacatgaaaaaaaatgagagttaaagcctaactcaggcccacaaagaatcacaagcgcccatattcacagccgccgaaaggtttcggcccccagttggatctcagagcccagtgtgaaataggactgacttgtctctgatggttacgttacgtagggattgaaacaaacaggcagtgagccgatgtggcagaccggagcgcagaagcgacgacgaccatgaactgaaggcgctggcagtgggccatgcgcccagaacgctgacgaaccacgacgacgcggacgttctacgaggtcggtggggccgtcggcagcccgccggagcatattcgccgtcgacagaggcagagaaggggaatgtgtgggagcatattccacccgtgggaagcctcggagtcggagatgtgcagatctgccggacgcacatgcgcacagctgccgcaatagaaaccagtgtctttttattttattttataagaaaaaactagaaaagaaaatccagtcttctatacaggcctgcacagagagagaggagggaaggtcaaatcgtagaaaaaaaatgcttccttttattgttacgacattggcctgaatatatactcattgtttctttccttaacttcaacaattattgattttgacaatccattttttattttagttcgccaaggttttggttggagaagaatttaaaacctagccaagcagtcgagcacgagagccgccgccgccgccgccgccgttggcaggacctgctgacctctgacgaccggacacggacgttttccaaagtccaaactcggggtaggtaggtcgtgaggctgctgcgttcgccaagggaagaagcatctagttgaagctagaaacgggcaggggggaggggccgacgctatcggaaatctgaagcccagcacggacggacggacgggcaggggcggcggcacatcctcgtctgaccgccgcgccgcgtccgacatgcgcaagctgctgctcttcccgcgcccccacggcggccgggccggcagagcacgggacgcgcggggcgggcctgacgtcgttttcacgtggtccgtcaccgttgcttgcatagcgtacaggagtatagtcccatttccgaaaccaaaaaaagtaaacaaaaaaatgccgtcgcattagttggctggcaagggaagaccaagatggttttcaaatcaaacatgtaaaaaaatgtttctttcttttttgaacgaacttaccagttcgaattctttaccggcattattggtttaaattgttccccaagaaagaaagaaaagaaaagcggggggaaatatgccttccttccttcttgcgtcacagaccacgcgcggatacaccgcacaacagccaggcagcaagcagagcacccaccgccggaagcggccgtcggacagaacgacacgtggggcacggcacagtccgggcccgcacgtcatcgaagacacctgacctgcccatgcgtcttctagagaaaggcggacggacaggtcacccccgcacctcattccaggagttgcatttcgctttctcctttatttatttataccaataaaaaataaatccgccttttctcctccctatcgtgtgtcttcctctcgccggctttaaaaacgcacacaagcgctaaaaccctctccaccgtccacctcagctcccatatccgctcccctacctctccagcatcctcccgtctccgtcgtctcgtctccgctcctcacctcgccgctagccaagggtaactcctcgctcccggccggcccccgcgtcggggttttccatttactactactcgctgctccctcctgctccgtgctcagactcagatccgaccaaagcggtttcgctgactaaattctccttcgtttttttttctttttctggacggattcctggacgcaggcaagatcaagatcggaatcaacggtgagtctgtacccccaacacaaactcgttcttcctgctcggttcgttgggtctggactctggagtgatctgagtggggtctctgcgcgcctgcgtgctcaggtttctgaaggatcggcagtaaacc

SEQ ID NO: 7 - DNA, 2046 bp, Zea mays
  source 1..2046: mol_type = genomic DNA
aaatgaattaatatattatctaaattcagacgggaaaaagagagaaggaatcaggaaggctgagtgcaatattagtttttcattgagcacaatcttattatgtttgatgaacatcttgaacaaagtatgagtggaaaacaacacttatgttatatctgcagcacagattgttagaagtgaaggctaccttgagatggtgataggtctctgaacccatctgcattgcttcttcgaagttcatagcaccaacaagaagaatcataatttcctacaaataagaaatgtttatagttactccaaaactacagttaaacatattgatatggttgatatacatgaacaactaactgtcttaatatgtttctgtttaggcaaaagatgacacaagtggacaaagtaagggaataatgcattcaacttctcagactctaaaggtttggatgaatacttgaatgggaagaccatttccagcatgctttccaccattaatgactgtatttgcagggacaggaagagttgtagcgcttttgccaacaagatccgctatatgcttgtagagtggaacctgcacagcaacaagcatccaatgatctgagaataagaattatgtgcggtggcatgacacatttaccaaattatgatgattttagcagaagagcagcacctctttttcagcagcaccagctttgcaagctgcaattgacactgccagcatagtatttgctccaagctcagcctgcatttcgcacaatatcatatctatttccatttggataaaatctatttgccagaaatatgggaacggaatcaagatacagaaaaaagttgaggctgtagtggtaagtgggaacacaagtacagtgaaaagatagcattattttccgaacagtcaaattaaccgaaataagtagtatcacatactgaccctaaattgactacacccaatgtaaagaattgcttgtgcatattgagccatcagttgtttgtttgtgtaatggatttgacaatgcagaattcaggagcaaaaaaaaccgcaaaggagagagagggaaaggatctccgagtatcccacctgtggcgcgccttgtcaagtccatgatggcctggtcgatctgcgcctgctgctgcggatccatcccaacgagtgcctccgacaccttgtcgttgatcacccgcaccgcgtaggcgacccccctggcgaggaacttccgcctctcggaggccccggccgcgtcggcggcggcgcccacgagcgcgcccacgctagccgtagatgctcggtgcacggccttgttggtgtgcaactcgacctcaacggccggtgcgctacgcccgtcaaggatctggcgcgcccgcaccctcgtgatcacggtgggcgcctcccgcctcatgtggccgacgatgaaaagcgccgggtcaggggccttggcgcggagcgccgtattcagggcctcctcgctcttgcgcgagagcaggtgcttccccaggtactcctgcactgacatggctgccacagcggtggaattttcttgtcctcacgacttcgccacgtcgtcctcacctccctggcttcccacgactccgccgtatcgaggtgctcggtgggtcgccttaatccgaagtccgaactaggaagacgagacagagaggtctgaggaatgggcctcgtttgatgttgagctgaattatttttccagcataagcccaggtcttggtccatgaacaaaattactagaaacccagcccagtactacgatctaaaagagggactgccactggatagctctctctagcattctccacgctccaatacagcggcgtagggtctatccgggtctatccgcgaacacgtgagaactctccagaaactgctttctcctccacttcatctctctcgctttccctctataaaaagaccccttctaggaattgagggagacagcaagcagcgatccgaagctcaatcaattcactcaaacctcttccccaaatcttcgattagattctcgttgacaagaagactataaccgaacctgaccgtaaacc

SEQ ID NO: 8 - DNA, 2489 bp, Zea mays
  source 1..2489: mol_type = genomic DNA
tagtcaccacaccatgatcatcttgtttgttgtagaatacccacttgttacctacaacaatttggtttggacgtggaactaaatgcaagaccttattcctcgtgaagttgttgagcacctcttgcattgccaccacccagtctggatctctaagtgcatcctctatcatgtatggctcaatagaagacacacaagagtaatgttcacaaaaatgagcaacttgagagcgagtgattaaccccttttgaatgtcaccaaggatggagttgacggggtgatatcgctgaattgcttggtgaactcttgagtgaggtggtctttgatcttgaatctcttggtcatcctccttttcttgaacaacttcatctcccccttgattgattcgttcttcttgaggtggctcattgtcttgaacttgatcttcttcttcttcttgagctggttcctcatcttaagttggtggagatgtttgtatggaatatgatagttgatcatgtgcttgtgaaggctcttcgggtttttgtggacacatgtctccaatggacatgtttcttagcgcgatgcatggagcctctttatcatctaaatcatcaagatcaacttgctctagttgagagcctctagtggacacacatcttcgggttaaatggttttatggaatattttttcttcacagatacaaatagtatcgaatatttcagatatttcatggttttgtcgaatacaaatataaaatcggatagagaaaactaaatttaattatatccatttccatccatattaaaattgaatacggatatagatatccatattagcattttatttgaatacgaatatatataatttggatgtctagacattcgaatccatctctaattagtgtgggaatgagggacactgaaaaacaatgacgtgcatggtgacatcatacaatagtacaattctgacgacgatgaaggaatttacgtgcggatcagcgacaccacctggtttggtggttcctgtcgccggaggcgcaagaaataaagagggcacataactacgtgaaatcaagcccaattagtctgccttggctcccttatgctgttttaaaaagtttagggttaagtcattagtagactgttgcgggtttagaaatttttagagaattttgtttacaacagcccctaaactaaagtttttgggaacaaattttagtagtcttttaagttgctctaagactatatttttttagttgaggaggacagtgacaattttggagttgctctaaaaccatgttttttagtcgagtgggacagtggcaattacttaactacaatgcacaacaccaggaatccaggatgaaaaattactacaccgagggctagtttgggaacctcattttcccaagagattttcattttcccaaagaaaattagtttatttttccttgggaaaatagaaatcctttggaaaattggagtttccaaactagccttgattttttttcctaagatatgtgcagatctttctttgagaggacacaaaaaaaatggattgggattgggctcatcgaaggccgaatattcctatccatcgttcgtgccggattaggcccagaaccagaaaagagctaggccgggctgcagtagcactatcctctgtttcacaatattatgcacttttgatcactttatttatgtcaaaaatacttgatacatcacgttttattttcactttcgcctctataaaagtattaagggatttctagacaattcaaaatgtaattttataagactatgtttgtcattttataaaaaaaaatagtttgattattttggtgaacgtgccttggtcaaaatttgtgggaacggaggatgctatcaaattcgtctgcgcagatgtacgcccagtaacgaagtatcgtcaatcgactgatgaccccgtcaccgtcagcaagacagcaactcaacattcaaattcgaccgtaataacatccacatacatacacggagtatcaatctagactagaggagacgggtgaacgtggtgagcctccgcccataatgcaaccctactagtgctagctttcggccgcgaaaaaacgtccccacccccacgtctcaactttatagccgccccctccccaccgccgcggccgccacgcgcagcagcaacccggtagcaggagcgcagccagcaagctcaggcccccagccctactgccaccgcgccgaacgacaaggccgagccggcggagcaccgtccagctgaggaggaggaggccgcggcggccggcgaggatgaggacaccggcgcccaggtcgcgcccatcgtgaagctggaggaggtcgccgttaccactggagaggaggacgaggacgcgctcctggacttgtgaggcatccgcggccgcttcgattcccccccctccccgatccgatttgcccatgtcttgttgatctgatgtgcggcggctgtgcaggaaggcgaagctctaccggtaaacc

SEQ ID NO: 9 - DNA, 2320 bp, Zea mays
  source 1..2320: mol_type = genomic DNA
tttgtattgttgtatgtaatgaataatctttatatatatatatatatatatatatatatatatatatatatatatatatatatatatgtgtgtgtgtgggggggtgtgggggtgtgtgtattgatgtctcttggacaacaagattacacaaaacacaattagaataaaaaaatatcctcgtgtatataaacttgtccgtataccatattagaacacataaattttaggcaacattttttccatcaacattcttcaatcatcaaccaaaatttacggatacacaagataagagggggtatgtaaggttgtacgtaatgggctacatgataacatcaaagattatgcaagcaaatctcaatccaccaggcgatcataaacatcatagttcacatgcttcagttttaaagccatcgtagttcccgtgcaaagacaaaaacattagaattattcaatacaagttgcacaagatagttcaataaaatttaaaccacaatagtattatccaacaaagctagttcataccattatagtttctagtaaacaagaatagagaacatatattaagcaaacagaccacataggataaggctaaggatgtatttttgtctattgttttctgtgacatcgatctcgtttgcacgagtaaaactaaaacatgagaaaattccgataaaaaataggaatctagaaatacgaacggaaaacactaaaccatttttattcctgtttctaaaatatatcatctcgtttctgtttttctctgtaaatatgaaaacgatcagatcatagttaaaacggaatacagtaccggacgaaacaatatttctctctcgtttcccccttatagcatctccaaaagctccccagaagtctcccctaaatctatttttttggaaaaacacaaaaacatgtctccaacagttcctctaaagcgcccccaactttttcatagcccttaaaactccctcatttgtagctacaaatgaggggttttttgggctccccagaaacaaactgctgctttaagatgtttggttgaggagccaagtagaatggagtcgtttcatccctgattctaggaacggagccgttctgttctgtgtttggtaatctggaacggagcggctctgttttttgtttggttgcagagtgaacggaacggagcgtgactgtgagagcgggatgagaacggagcggctccgttcggttgattttttggagcggaatggttccggatttgaggagaatattccataattggagtcattccgttctagttcctttataaccaaacagcaacaaaactgggatagaatggttccgttctacttggctcttcaaccaaacactaactaagggacctgttggagaaatgattaaaatttaccctcacttattatttagatattccttaaaactaattttgagaagtcgttttatggagtgctcttggagatgctcttagttcgtagatctgattgtgtgtatgtatgtaagcatatgcgtctgtactacgatccacggtaaaaaagtcacaaacatataataatagcgtcattcaataaattgataacgtaaagtataaatgaacttttaactctgtttgagtaaacaactcgtgcagagactgcaatgaaatctattattttttctaaaataattatacaagttgaggaaatgctatttgttccactaagcgacgatgtattttgttttttaaaaaaatcgacgaggtactgctcggttattattttcacatgcaccgcgcgttgtttttgggccggcccatttgtattgcgaatttgcggagacgaatatgaccgaatggagtttagaaagcccagctcacttaggattgtctattttctcaagaaaagagagaatgggcccaaaggcctaaacaccaaaacccgatccgcttatgggatgtcatggacatggagtctgggaccgtccggcagatgagacgacagccgtcggatcagaaaccctagcgcgggaggctctccctattaatacccaccctgcaccccgcgggaggagtctttcctagggtttcgtagcttctagccgccgccgcgtccgcctcgccaagcgcagcagccgccgcagcacatcgctctctcgatctcagccatagcggaggtggagcagcaacaggacacgccgaagctcttcaaccgctggaccttcgatgatgtccaggtacgcgaacgagtcttcgcatctctgcatgcttcgattctttagccttgccgctactagcagtggatggaaccgacgatgaaatctgcaggtgaacgacatctcgctgtaaacc

SEQ ID NO: 10 - DNA, 2686 bp, synthetic construct
  misc_feature 1..2686: note = Includes maize-optimized Kozak sequence.
  source 1..2686: mol_type = other DNA
ggatcctgaggtgtggcttgtatgtttcctgaccccctggtggtgttcccagtttgcaatacatcattttctgtagcccttgtacttgctgtgatccctgtttattgttcagggaaaacttagtgcagtgtattagaaatatagaaacctcacatttcgaagagcagaatagtgtttgataacagtttttttaacgcaacaacgtttgttagactgtgtccagcggccagcgggtagtgtaaaataggtgacgcgaaactatatgtgatattgtttgacactatttgcagagtatagtttaaaatagggggtgtgagtaatctgctgaagatagccttaagagttaagataccaaggtatagtttatcatatgcaaaagaaaagaaaaaaaaggaaatacaaaccattgtttttatgttggttagagctaggtaattatctcttgaccagtgtatttccaaacctgttcttctcagtttctgtgctccgtcgatactgaacattgttgttcatttctcatttgttgaacgatggtatttcaggagctagagaagaggagagaggagcttgagtctctgttaactgctgaccggatccgctgcgtttgggggcggcaggatctccgaccggatccgcccgccgtgcccgcgaccggatccgctgcgtttgggggggggcaggatctccgaccggatccgcccgccgtggccgcaaccggatccgctgcgtttggggcggcaggatctccgaccggatccgcccaccgtggcggcgaccggatccgctgcgtttgggggcggcaaggaagcgggtggggcttctagggttcaggtggcgggcgtcggggtagagagtgcctgcggcgattctggcgggcatgcgcagggtggggcaggggtgtggtcgagcgggccatgcacaaccttccatccgcgggattttgcgggcgttgctggcgcgacggatttgcaggagttgctggcgcggagctttgagggcgcggccgatttggggactgcgggcgttggcgattttgcgggcgttgctggcgcggcagattctgcgggcgttgctggcgcggggactgcggacgcgacagctgtgctcgcctgcggccgcgaggaaggcgacggggactgcgggcgcggggagggaaatcgcgggcggagcgcgcgatggcagaacacacagaactatggacgcctacactaagggggtgtttggtttctagggactaatgtttagtcccttcattttattcctttttagtatataaattgctaaatatagaaactaaaataaagttttagtttctatatttagcaattttagaacaaaaatggaataaaatgtagggactaaacattagtccctaaaaaccaaacaccccttaaatacataagaagtagtagagattattattatttgtgtctattgatatgatcattatgatattatattttactgttttaatctaaatatagttattatattcagataattattttattaaatccaaaaatatttaacttctaatgtaataaggagagaatactctaatatcataataatttcttctaaagtgaccctcggatttggagatgactgacagggagggctgtgcacccttctttctttccttttcaattgaagaacttgggttgtgcttgctcacacaaccgaatgaccgatgcacaccgacgcgcacggagcggaccagcacagcccggaaactgccctacgccgacacgttttccggtgcggcccagcagcagcagggaggaaggttcgctcggtcgctgggcctggctccgcgtgccgtccctcctataaatgcgagtcctcgccgagtccactgggcaccagaagctcactcactgctcgttgccggctgcccccgcggccccggccagtccatcccctcgctcgctccccaactccagcaggcagatcagatacatccatccattcgcgcaccggaaggtgagcgccgtgaacgaaccatccgccctgctagctgcgatctgtagccttgcgtcgctttcgcgcctagatcgtcacgtcacctatcacgatccgtgcggttctagatctgtggtttttccttcccctggtggtcgaatccttccatccaccagaccaccacgggacctcgtggattccttttggttttcctgtgccgagagccaaaatcgagggggggggcttgttttttattggctcggtctcccgctgtctcgtgatctgatttgctgtagtaatcagcaggaaaggaagggttgaactaagagcgccgtggcggtttcgtcgtcgctgaacccggacgcgccgctcttcatcccggcggcgctgctgcaggtggaggacttctcgccgcagtggtgggacctcatcaccaccactgcctggttccgcgaccactggtcccgcgagcgcgcccacctggacgagatcgccgagcagatcgacgcggccggcctcctccccgacgacgaggacctcttctacgacgaccaggtcgagcagggccccgtcgccgccgcccttaagataggtactgatgtctctctctctctctctctcttactctcccctcgattttagatctgcctgaaggacgaatcatagtgacctcacgttggtgcgtttttctccaccagattcggtgctcaaggcgctgtaaacc

SEQ ID NO: 11 - DNA, 7672 bp, synthetic construct
  misc_feature 1..7672: note = Includes maize-optimized Kozak sequence.
  source 1..7672: mol_type = other DNA
tatagaatagctcactatcctatttattatagtttaagtatatagccaatattttaaatttactatttattaaattctagggaagatagtctcaattcataactttattataatacgtttgaaattttaaatctttaggaaattttcttaattcacctagatacgattctggagtgttacaagctgcgaatatactggtgccattgagtatacataaatggatttaggtggtgctcaataggtgaaaatgagatactaatcacttaaatttcaaaatttctatggtgccactgtactcggataggtctatctagggctggacaaaatgctcgtggctcgctggctcgctcgtttcgtggtcagctcggctcggctcggatcggctcatttgaattttgtcacgagctgagctgacattctagctcggttcgttaacgagccagctcgcgagctaaacgagctaccatattctagtaaaacgaaattatattcatatcatttatagaataattgatgaacatgttatatatatgtgagatgtctatggcctatgaattaaactaatgattaatgaactatgcctatgtgttaatttggtctatgcaaatataattatgggttaaactgatgaacatgcatgtgaattgtgaattaatgagtgatgaattgtgctaatttggtgttatattgacatggtttgtgaaactatgagtataattactattttctattgttaaattagtttgaaattaactaaaaaataattattatatacattttattttttttctgctctggctcgcgagctaaacgagccagctcgacctcgtaaacgagccgagccgagctgactctgtggctcgttaccttaacgagccgagccgagctggctcgttagcttaacgagccagctcgaactcggacgagccgagccgagctggctcgttatccacccctaggtctatctagcttctgatgtttgcaaaccttagagttggagtgttcagccagctactcctttgctttgctgaataaccataccaaacacgcccatattaatacccgctcggcggtggttctgcaatcaaacgcaggccgcagtcgcgtgcggaactagaggtccttcagagaagtgccgtgccagtgccaccgccggccgcatcatcgttccgcccccctggtacgagcacttcgcagagctgcaacctacatcccttttacataaatctattgtctcgtattgccgttgacgccggaatagtcttcgcatcccttttacataaatccgatgttttctttctccgattcctttgaggaatcatcacgggtcagggcaggtgttctgccgtttgcccttttctttatattctccttagaagaaatatttagttggaggctggacatagccggaggagctaactaatcgagcggtgtactggcaaaacaaaaggagcggagcaagaaaggggagaaaaaactagccactgccggagcgctattggccgtgttgggcctggaagcttgcatcaatacttccctcgccccgatttggttccaaaatcatacaagtcccaaagttgtcaagatattggaggtatgcaagcgacttggatctcaaaatagaagaaatttcggatctgagcacaaatctgagttgaaaaaactgcaactcaaaatcatcaaaaaaagaagaagaaagaaacgaatatattcgctcctcttctcagccgaacccaaaggaattgaatccaaaccctgggtaggcagacagtgagatatggaggagagcaggaggcgaacaagagaggctgcggccacgaatatctcacgaacaagcacatcatgggtccacggagcgggcagggtgacgggctcccgacggcgagctacatctcggaagagcaccagggcagcatgtcgtgttgggcaggttggccgtctggcggacggcggacggtgactcgtggtcagggtgcacctgctcgattaaggcgcctgactactcatgtcttggtctctttgcttgtgtttgctatatgctgctcgtacctcatgagcatactaagttgactgctcagtctgctgagtctgtttttctagggtatagtgctgagcacaagggatatcattgttgggatatgattgctcgttggatgagggtctcttgggatgttgtctttgatgaggctcattctttttattcttgtccttctttcgatgctttgtcaacatccttggttgatcccatctcttttctatattttctagatgcccgtgttactattggacctgcctcacgcttggtgcgcccacgatagtagccttagctccttctgacatgttcatctctctttcggtgccttcctttgtggtgccttctatagtgttttctttggagcctgctgctttagcccctgactacgctatgaacacttgtctacacccgccgggtcatcaattcttttggtacaccatcatcctctcatgcgttgccctcttatgatgtgcgctcttctgcaactcattcattttcttgcgatttacctttgactgatgctccctattcatctctggatccagcttcctcagttgactctttgctggagccacctcttagacggagtcatcgttttcgtcagccacctaatgggtactctccttcaggtttagtcgctaccgttctttctgagctgacttcttatcatgatgctattcttcatctgtaacgacaacatgcgatttctgaggagattgctactcttgagcgcactagcacgttggaacttgttccttgtccatcacgtgtttgtcctatcaccagtatgtgggtctataaggtcaagacccgttctgatggttctcttgatcgctataaatctcgtctagttgcccaaggcttccagtaggaacatggttgtggctatgatgagatttttgcacctgttgctcatatgaccactgttcgcactcttcttgctatggcctctgttcgtgcgtggtccatctctcatcttgatgtcaagaatacctttcttgatggtaagctacttgagttctatatgtagccatcgcctaggtattctatttctgcttgtatggtttgttgtcttcgccgttccccttatggcctcaagcaggctccacattcttggtttcagctctttgcttctatgataactgttgttggtttttctaccagtaatcatggtcctgcactctttgtgtactacctcctctcggggtcggactcttctttatgttgatgatataattatcactggagataaccttgagtatgttgactttgttaaggcacgtcttagttatcattttctcatgtctgatcttggtcctctgtgttactttcttgggacaaaggtttcttctttgtctcagggcctttatctatctcaagaggagtacattcaagattttcttcatcgggcttctcttaccgatcactagattgttgagactcccaagcagctcaatcttcaccttagtgccgatgatggcgagtcttttcccgaccatactcgttatcgtcaacatactgtaggaagttttgtttatctctgtgtcactcgtcttgacatttcatatgttgtgtgtatcctgagttagtttgcttcagatcccatccaggtacactatagtcacttgctttgtgtcctacaatatctttgtggaaccatatctagatgtatgttctttccacattctagctcgttgcaactgcaatcttgttctgatgctacttgggctagtgattttttcgatagttggtctctttctcaatattgtgtttttcttggtggttctctcattgctcggaagactaagtagcaggtagcagtttctcgtttgagtaccgaggctgagttgcgtgctatggcccttgtgactgcagaggttacttggttacgatagttgcttgaggattttcatgtttctgtttccatgacgactccttttgtctgacagtacaggtgttatcagtattgctcgtgatgcggtgaagcatgaggtcaccaagcatattggagttgatgtttcgtatacacgagctgaagtctaggatgatgttatcttgatttggtatgtgcctttagagcttcagttggctaatttcttcacgagggcacaggctcgcgctgagcataaatttttcctctcaaaactcagtgttatagatccaccttgagtttgagggagtattagatagatatgggtttatttgtatttttccattttataagggtattagatagataggcaacgactgctatgcaagtagtcattctgtgcaagcgtgcaagcaaaccatctgatccattatatcgtgatccaaccgtgggtcacatttaacacttaaacccttccaccaccaactcaataatctttataaaaaaacccctaacaaacaatggttatatctgtggttggatcgtaatctaatagatcagatggtttgcttgtacgcttgcacagaatgactgcttgcatagcagttgttgcctagatagatatgggtttatttgtatttttctcttaagggtttttgtgtatatttgtactcatgtacctatatatttgtgctagttgaccccataatgaatagacctgctattcataatatttgcaaaccatgaaaatttgattattacgaactatccaaatactcgaacacatgggcattatagctcacaaaaatggaaggttgagctgctgcttgaagaacctcaacatctttgaacaacaacctcaacgaaacttgtatatgaaccaacttccaaacaatcccttgtggaaggatagtaatgacttcagggcattgatcacacatatccgacggtggaactactgtaacaaccctcttttctgtggaatatagttgaaactctacaacttgaccaaaaccaagatgacgacatatggtggaactaacaaaacaagaggactacactacctcattagcttattaagcacaatctcttggcaccacaacaacgaacaacaaaaccatcatttggatgctctgtgggcgactaaatgcaaattctttgcatggttgatcatcccaaattggtggcacttagctataggctagcagtgagaggatggccgaacaacatgcattgtccactatgttggtgtagccatgagaccaaccaccacataaatgccaaacgttcattcaccaaaaaaatctaggcaacaatggcttggatttcttacctgcagctccaccaagctaactggagttcaattaggtcaacgtatgggtggtggtcgagtatagcagtcacaaatgatgttctaaagatggggttgtgttaacacatcttgcttgtagcacgagaacactggaaggagtgaaaccaaagaatctttcaacacaaggacctatcaacgctatccatgattgggaaattcaaggacgaaactagaatttgggtgaacacatgcacaaggcacctaggagagcctttcttttgtactgttaatccctttttaaactctctctgtccttaggagttcgtttcttccgctctattcaatgaagttaggcacaatcttgtgtgatttcattagaaaaacacaagtaaattgcatggtcagtacttgaagtattacaggaatctcgtctgcccccaaactattaaaccttatatttggctccctaatgtacttaactgatctcattctggtcaaactaaacatggtgatggcaaggagccgatatggtcgcccatgtggatgtgatttaagcaaaaaatctcatggtccatagctgtgtcaacaagccaacatgccatcgcttccttatgccgagactgcccatgtcgctcgcttttactgtcatcatcatcaaactgcctgtcatgtctacggatgccatgaccgctgtcacacatgatgtggagatgaacctgtccatcaacttccacgtgctgccactatcgctagctgacaccgtcttggtcattgctgtgtagggctaggctaagagtcgctgaatgatcctttcgctctcctttacaggaacatgctgtttactttgtgtcgccaaggcgtgctagagtacctcttctacacctccagcaccagtagccttattgttagcttgcacatcccacataagcaggccgatgtgaatgataacttcagggacgtcgacggcatgtcactgccaagagtcatttggtgggaagcgttgtcatgccatctgtcgtgccattttgtcctcagttcgaccgccattaccgtgagcacaacctttgcgcatggttggccgcttccatcacccttattccgtttcctcgtgttggtcttgccccaaggctatggttagcagaccgtgcatatggccggcaaaagactattttgcactgtagattgcactctttatatagtgaagtttaaaataggagatgagatgaataaggctgctggagatagcctaaacccttgcagctcgtgcttgcatcgggggagccaaaaggcgtccacctccaccatcgccgaagcactgagcactactctggcttgtgtttcagcaccacaccgcagagtgcttagggccaccaacctcctcttgcctctgtgcccagagcaccatcagctctgctgcctccctctgttccttgtgcttgctaggcaggcaattccgagctggggcccaacttgtaacgctgatttcaccatcttgccactgccgggcaccaagtggacacatttgacttggcctagtgggttttctgcataaatcacatacatgtggatgccatatcaggctctttggtgttgtcgtgtctactttcgacaaggatgagatcacttaaacatattagggagccaagtatgtaatttcatagtttagggacctacacaaaaatcgtataatactttagaacagccgtgcagtttactcaatcaacacatacaaagtcagatcttaagctctgatacttcaaaggaatggttgagcccagttgacaaacaatcttgcttcattcattgaattgtttataggagtggctatgtaactactgggtggttttgtttgacctgtcatccaaattgtgtagtcaaccataaacatacacgtcacacaatacattttggatgtgacagataggatttaggcgagagaatgtacaatgtcactgaaaaattaccactgtatggaaaggacaatctaagtgaaaagagaaccagggcctaatggtttcaggacttcaaactccggccaaatgaatttacagtgcttaaattaactcatgttaatcatgatagccaaagcatgggcaaaagagaaactatgaataaatcgacaatgtattctatatagcagtaatataccatgtcacgagcttttacactaatgggctgtatttttctgcagttattttaactggcaatattctatgtcacagtaatatttgttaaattttttccagaatagcaactgaactagaagtctagtatttcttaattggataacaaaaggaattagtgtgcatttggcttacgaacaatcagtcacccaacattgaatttgaagttctgtttcctctttgttcagacgacactctccaaatgaatgccttatattttgtgttgctcctcttttctgcagagtgttcagtaacttcttccgatgtaaacc

SEQ ID NO: 12 - DNA, 1014 bp, Zea mays
  source 1..1014: mol_type = genomic DNA
aagttcgtcatgttctgttttggtcatttgggcaccaaagtttgcgcctcatttggttctgtaatccgtgaggtcgtgcatgtacttggcgtattgcatgcagtgaataatttagcttgggtttgtttgttgggggcagtgttggggacggatttggattggggtttatgcttggcatcgcgtcgtatcgaaactcagctgctgtttcgctgagtaatgtacatttccctggtaatggtacttgtggactctgatgcttttatgggaacgagtgcattttactgccgtgcttggccgtgctgttgaattattcccgtagtgtattaaatattggatgcaccagattgtttgtcccatttacgcataattttcccgtcatatgttgaattatttacgaattataaaatatgtaatttatatttatttttatgaagtatataaaattttacttttttgttatataattaaatattttttgttttgataaaatggtatgttttattttaaataaaaatctataaaaaaagatgatatggtggctggaaggtaacgataaattatgtatactaaagtattaacgatagtaaatatagttggacgatagatgatgtgacggggtttgttcttttgattttaatctataatgcttgtctatagattacagccgcatcaatttaaatagctgcttttaatctgaagcgaacaaccgataaaaatagacattaggccatctccaacagttcgaccgtgcaaagtattattttgctattgcactatttgaataatggattttagaatagggagtgataaaaagtgagataaaaagtttaataaaaatagtcttagatccaacaaatatacgctgccctcccatcagttgcctggacccatgccaagaagtgccagccatagtcgtatgaaaaatgattctaagggcgttcgcaatggttgtcttaaatagttagctattgtgaagctgaaactaagagaagaataaaaatataatttatttca

SEQ ID NO: 13 - DNA, 1216 bp, Zea mays
  source 1..1216: mol_type = genomic DNA
tacggggaaagaaggagaagaagaagaagaagcccaggccggagaaccatcgcctgcatttcgatctgtttcaccgcaattcgcattgttagtcgtgtattggagttatgtgtacttggtttccaagaactttggttccttctcgtttttttttccttgcttgtttgagcgtttttgggcagcgctggcctggttcctagtatggtgggaattggctgcaccttttgcttcgaataaaaatgcctgctcgttcacctgtcttccagagtgcaatgcgatgttctgttgcccaggtcgtgtggttctgactgatggcgatgttgtgttcttctgttaatcgcctgttttaacgtggtaggctgatgcttgttcttgttgagaaagcttgctgtgccagacatggctgcttgaatacaagtgaaggaaaaaaaaagccatgccaagtaaagttgcacaaaatttcaactgctcagtggaccactggaccatgttcttggttattgcagttgcagggcttcacatggcgtttggacagcagtcttggattgatgcataaagaggtggtggttaatgaggacgcaaggccgttccctcagagtcagtcacaaggttgcagaggtcacggttctcttccctttccgcttcctgtcacatcggaattgttgtttacgccatctgcccatcacccaccaagtctatgtttctgtactggatctttcaatggcggaacgcgcttagttcttcgtcacagtcgaatcacatgatctaatcgatgtctttaatctcgctgtaaaaagggtgggacggtgggtgcagggtagggaccagggaaggcctgcctaaacgtatccataaacatgcacagcaaccctaagatattatactgcctacttcctaagatatagttatttctagtctattttttttccgtccacatccaaataagtgataatatatagacatacatatatatactatattcatcatagattaatgaacgaatgtatacttagtttaaacctaattatattttaggaaggatggagtatgaaacatgacaatacaacaaaaaaaaatcatgtaattgcatatcgtcaaagttatctgaagtaaccaatccagggggaaatcccgttagcaaacatacaagagcaccgccccactacatcccagaaaataaaacaaaaccagaactcagatggataaataatactac

SEQ ID NO: 14 - DNA, 1216 bp, synthetic construct
  misc_feature 1..1216: note = Comprises two mutations to remove unintended ORFS.
  source 1..1216: mol_type = other DNA
tacggggaaagaaggagaagaagaagaagaagcccaggccggagaaccatcgcctgcatttcgatctgtttcaccgcaattcgcattgttagtcgtgtattggagttatgtgtacttggtttccaagaactttggttccttctcgtttttttttccttgcttgtttgagcgtttttgggcagcgctggcctggttcctagtatggtgggaattggctgcaccttttgcttcgaataaaaatgcctgctcgttcacctgtcttccagagtgcaatgcgatgttctgttgcccaggtcgtgtggttctgactgatggcgatgttgtgttcttctgttaatcgcctgttttaacgtggtaggctgttgcttgttcttgttgagaaagcttgctgtgccagacatggctgcttgaatacaagtgaaggaaaaaaaaagccatgccaagtaaagttgcacaaaatttcaactgctcagtggaccactggaccatgttcttggttattgcagttgcagggcttcacatggcgtttggacagcagtcttggattgatccataaagaggtggtggttaatgaggacgcaaggccgttccctcagagtcagtcacaaggttgcagaggtcacggttctcttccctttccgcttcctgtcacatcggaattgttgtttacgccatctgcccatcacccaccaagtctatgtttctgtactggatctttcaatggcggaacgcgcttagttcttcgtcacagtcgaatcacatgatctaatcgatgtctttaatctcgctgtaaaaagggtgggacggtgggtgcagggtagggaccagggaaggcctgcctaaacgtatccataaacatgcacagcaaccctaagatattatactgcctacttcctaagatatagttatttctagtctattttttttccgtccacatccaaataagtgataatatatagacatacatatatatactatattcatcatagattaatgaacgaatgtatacttagtttaaacctaattatattttaggaaggatggagtatgaaacatgacaatacaacaaaaaaaaatcatgtaattgcatatcgtcaaagttatctgaagtaaccaatccagggggaaatcccgttagcaaacatacaagagcaccgccccactacatcccagaaaataaaacaaaaccagaactcagatggataaataatactac

SEQ ID NO: 15 - DNA, 1004 bp, Zea mays
  source 1..1004: mol_type = genomic DNA
agagagatatttctgcctccctatcgtgggtcgtccccaatggcctttggtcgcagaccatctttgctgcttgtctatgctgagaataaatgtgaacggtgcccctggacgctggatccatgctggttttggacacggttgtctttttgtgtttaacttatctgctaccgtcctgtaacgaattcactaagttctagttcttttgtgctttgttaagaatataaacaatgaggtttcaattttggtggcgccatccggtctgattgcgtactagtatcatgcatatctgagctggtctttccggagtaacagtttttttttgtttcgtgtcaatgattctcagcctgttcatttggtcttaatccatatcggttactactataatgttgtcttgtctctatagattgcagcagtctaacacatgttcttggtatccttttgggtgtgtttaatgctatttgctctgtgttgtctttttcgttaatcgggacagagttttctctctgttttgacagtttcggtggttcttttttgtgtgtggtcagcgagtcacgaactgctggttggccagcgaaatagcgcagactattatgggtcttctgctggtgaatatggaaggacgaggttaaattttctgctttgatgtgtctgccctctgccgctgtgctgcatgcatgtgtttgggagcgaagcagcgtgctgtttgggcgtatggttggcatggtgttaaattatttcgctgattcagctttgggttatcactggttttgatggtcacgctgtgtcgtaagagagatcgtggcagcaaaatggagaaaaaggcagtgaaacaaactttcagggtgtggaatgtatgggattcgtatggttgaggccaggatttgtcaattggtcaagttgaaaagtaaaggcgagacagattggcgaggaccgaggagagattggggttcagaaacttcggttacactttaatcccctccatctccctccactcctcccttcta

SEQ ID NO: 16 - DNA, 1023 bp, Zea mays
  source 1..1023: mol_type = genomic DNA
acctcggtctgcggtcgtcgtacctgcgtggtttgaggaacggcagttcgcctcggtcgttctgtgaaataaaattgggttacaagaattatggcgtttgtcaatatggtcgtaatgtcgtaggatggtggaatgtggtcacaaactttgcgtatgttgggtctactggtggtgtctgaatctatgtatggatgtcatgagtttgtctactagagttctagttcctgtggtgttcgtatgatgtatatcatgagttaactctatctaaaatttcttcattatggtatttttaagaaataaattctttattatggtttttttaaagggactatgtccatcctaacaagagcaacgtccaataattctctaaaacttaaatttaagaaccgaataaaaaatgagtagtttttaaatactttctatctaaccttatttactcttctccatattttagtagttttttaaatagacttactaaatttagttactctatattttggtaactcgacagaatacaatctgtgattaatttttctcacatgtggtagctagatacgacacttttttttttactttttagcacatgcacaatggagtagttagattcaacacttatgacacaagttttttcttcaccgttggactattattgtcgcatatgttgccaccgcgaaccaccgctcctctataggttatgtcaatcgacctttcctcttttgtcttcgacatcatccaagactaacaaagttagattcaaatcaaactagatgtccagactggcaaagttagatttaaaatcaaactactatctacatataagcaactattagggactaagttatttttctacttcaatagttgttgctagcaacttgctaaacataattttagaaaacttttttagagaactattagagttgctctaaaaggttttgtagtcccatttactgttttgtgactattattaggtggagtttgggcagtaaaaaggccaaatagaaaatggggttgagattgggaaaa

SEQ ID NO: 17 - DNA, 1028 bp, Zea mays
  source 1..1028: mol_type = genomic DNA
aggttgaaggcaaacaagggtcaaatggatgccattccattcatttcgtttccaaggttcagcttccccgcaaattttcattgtgttttctccgagatgaatgtttgtgttcggtgaaatcagagtcgtcagtcatctacatagcttttcttggttgatagactgttattttaagtcgcatgtttatctgggatggctggggtcagcatgtttgtacaattatttggagttgcttttggaatggtcgcggtttgatgagttgcctacagccatgagatgtcttttcgctccacttttatggttcattcgttctcaataatatgggatgctatacttgtgttgatccattatcttgatgcatcgttgtctgtgcactgcaacaacaatacccatctgaacacccctatcaataaaataccattttttttctttccatcccactaatctaggcccactttctcactcttttcttatccactatattgtcaatatagaatctggggagagagagagagagagagagattgagagagagagagagagagagatttaggctccctcccttcctatattcaaaataggtatcgcctttgggtcacctgttggaatgatttattttagtatcgtccatatttaatttaaacaaatgttgaattttacatgcatattgatttaaagtttgttagtttatgagcatcaccaactaagatctctaatgccaaagagcatctccactagttccaaaaaactctctaaatttaatttaggatgttagtaaccagaaattatgctccaacagtttcctaaatgagttccttaaatataccaactttttaaatatctctatttagtcaaacttgagaaattgtttcacactcccaatagttgtcatcaatcacatcgtaaaatcatttgagttcgcacccgtgtacaagtggtgcactttaaataaattaaattacgaaaatgataaatttactatcgagttagcataatttaaaaatatataacaacataagaactgggaa

SEQ ID NO: 18 - DNA, 1002 bp, Zea mays
  source 1..1002: mol_type = genomic DNA
actgagcttgtatcctggtgcactctgcgctggaaacttttatgtcgctggcagtcgtatcggttcttgttttaccaatgtttagagttttttgagacctatatgcggttttggttttcagtgcacaattaaaattactgagtaatgtagttgattgggaacagaaatgtttggtgcctggtttaccgaactccagttctcttgtcatttttctttattctatagtctgtattatgtatgcgtatgagtattgagatgattctgcatttgaattgtctgctttgttgctgtcgctgtatgcgtaccaatggtaacagggtagttgtgggaagtagacacggccggttctatgttttcgtgcttccgcggttcaggctggttaagcctatggagcgtacgcacgctcctcccgtctctccgtgtcctgcatgctggcaacaggagtgcggcccagcgcacgccctaatcgacggcggtatatttgtctgtccctccattttgtggtgaggctattcgcaaccgttatccttaaattttttctcctatatcactattcccctatttttccttatattttttcatcttcagcagcggttctcctaaatactccctctatacccactacaactataaatattattttccatatctattcatcatttattaccactttttttcaactaaaaaatactcgcatgcatggattttacggaaggggggctgtcacagtatccccttgatctgctgtgagagaaaagggggacactaggtagggtgcaaggtagggagcagcggtgcgggtggtagcgtggttactgcagccgctacgacgtgagcagtgttaggggagaggatggaagggtggtgcgctgctgcagataacccgagcgccaaacactcatgggtgataattaggtataagaaaagatattttatggttaggagagtatagagagaatttagtggtaacttctatggaagatggaaaaataggggtgaaat

SEQ ID NO: 19 - DNA, 1001 bp, Zea mays
  source 1..1001: mol_type = genomic DNA
gttcgctgggggaactcatcaggaaggctgctgcccctcttgcagccttgctcctggctgccgccgctgtcgtggtctgctctttcaagtcgaagtaacggtggttcgagctagtggatagtgtggctcaactgtagaagttccttttgtatagcaagcaagtaaaaaaaaaaaatgaccaaaaaatataacaaaatgcagctgtaagtttactgctgctctctaagtcgtgttcagtcatccagtgtgtctagtctagggaaaccccataaaaatggtgaaggtggaatcccatcccagtgtcataattaaggatgcacttcttctgtaagcaaatgtatgatgtacaatggccggccggcagtctaaatgttacaactagctcttcttggtgaattcaccggtccacactgatgtgctgctatgtatcattactatccagttagggcttgttcggttattcctacgccatatggattggacgggattggaaaattttagtagacattttgacttctatggatttaaacccacccaatcccctccaatccacatggattgagatgaaaccgaacaagcccttagttggatggatggatgatctcacgcgcttgagtttatctagttgctgatatgggagagcccctcaacacctcaaatattgtggataagtttaatcctacactgtcagtcttcagttataaggcacgcactcttcgacgttgggcgctgtcttttgtttcaaaggttgaggcaacctcaacctcgtttaaaacagagagcaaagctaatttccaaactgatgtaagtcatgtgtccttaattaccaaagtagcaatgatgacattgttatgttgtgtgctaatgagccattacgtgaacatcagttcctgccttgccgcttcgttcagccgggccggggtatattttgagtcactaaaaggacacgtcggatgaatggaaaattgcctcttgtctttaccaagagtgtcgcccgcgag

SEQ ID NO: 20 - DNA, 2037 bp, Zea mays
  source 1..2037: mol_type = genomic DNA
cgacgtacaaatctcatctgtgccttgctctagtttcccaaatggaattaactatgcatgatttgtttggaaactcttattgcatccatccagataatgcatccaccataaggtaatatcttgatgacatctgtgcctgatggtgtaccaaatgtctctatctctgcattgagccacgagtaggaggatagcctaggggtgccttgactccaaagttgtattgaaaaagatggatgaagcaggcaaatgctgcctgaatccatgactcagggcacagattttccactcaaaggaagataagattgcattacttcatgatcttttgaactgcctctgcaagacgggactcggatagtggatgcaaagatctaatactggcctcaggcaacgagttgtttcactcgaaagtctagaaatgaccgggctcaaattttgcaccccaaggaaagtgagtttgcattacttcatgaccttttgaactgcctctgcaagactggactcagattacgcttgattggttgccggcctcaccttcgcctggcttgcgcgagcctgcgtctatagaaatgcgccggactcacgtctccgtcgatgcaggcattcgactgaaaaaacatttaaactgcacccatgcgtgcgggctgagcttatgtcatacaagtaaccaatcacaggcttaagttcagtcaacgcatgcgctaagcttggatgtggctgaccgggcaaccaatcacacagatagtggatgcacggatctaatattggctaatttggttaaacttgtctaaccttagacgtggcaagtgagtcagcggatcaaatctgctctaaaattgtctgcctcctagatgtccttggtgttccaagatttaatcatcactgcactatttctttgcgttgcttcgctgcagcttcgcgttacttgcattcgcttaatcaggattactttgatcaactaggtttctaacttctactaccttcacttgcacagggtgcccgtcctgctagccggtgtgcttgctgtgcgatcgtttggcatgtgcttgttgaggggttgctaggggattggagaggattgaagggattaaatctcctcctattcaattttgaataggaggggatttaatccccttcaatccccctcaaaccactagtaaccgaacgtggcctgagggggcgggcgagtctttatattgaatgaaactacataaaatagcatgccgtctctgtcactggcaatggacggtggtgcctagcgcaactcagcgcacaactgtgtgtcttgatttttcttctgtttatcacggcattagtgccatgccgttttatgttacagtgttgtgtgctcgcaagcatccgaaaatatgcgtctgagtttagggttgggtcaaacttgtcgaatttggggttctgttataatatgttgagcatgaataaagatggatgctggtgactctgtcgccatcgccgtccatcatgagtgtcctgtaattcaacttatatctatcatgtatgtatgtatgtatgtatgtatgtatgtatatgctgtctactatgcttctttgttttaactgaaatgtgtgttacagtgttacttctctggggtccatttaaaacggcatttcgtttacgataggaaccagccattataatctttaaccaataatttcgctaaccaatttcaactattgcaatgcgaacttaatattatcagatttataaccgaatgcgctatcaaataatcataaggttgtaatcataataatataatataaaataaatgagtgctcgaagtgaaattttagagagcgttataagaaaaattgatgtgatctccaagaataatagcccctcccggctcccggtacaaacatagggcttctttagaatgcaggattgtgagaacataggaataggaaaaatataggaattctataggaatgtatatggaaaacagaggattgaaaaacacagaaaaaatgtgaaagcaagtctttggatgaagcgtaggaaacttataggaataggaattcataa

SEQ ID NO: 21 - DNA, 16119 bp, synthetic construct
  misc_feature 1..16119: note = Recombinant vector.
  source 1..16119: mol_type = other DNA
attcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgctgcaggaattggccgcagcggccatttaaatcaattgggcgcgccagctgcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaactcgagacttttcaacaaagggtaatatccggaaacctcctcggattccattgcccagctatctgtcactttattgtgaagatagtggaaaaggaaggtggctcctacaaatgccatcattgcgataaaggaaaggctatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgaacaatcccactatccttcggtaccggacccgagatatgtgatgtatatgtgatatatgtgatgattatgtgatatattttttgtttgtttggatggaataataaaaacaaataaaaagggtatgttggccacttttggcgagtgtaacactcagcaaagaggtactttgccgagtgtcacagtcatatcattcggcaaagaaggcacacatgggaaccgataaagcttctttgccgagtgttgtggccttagcactcggcaaagaagcaacttttgccgagagcctcctagtgtactcggcaaaggaactgacaaaggagcccactgatgattcctttgatgagtggtagtccggcagacacacggcaaagataaagtctttgccgagtgccacctaatacgcccggcaaaggaactggcaaaggacacacgatgagtttatttgtcgagtgctagtacaatagacactcgacaaagagtgagcctttgcagagtgtcaccgtggcactcggcaaagtccactctttgtcgagtgtcaccgtgacactcagcgaagtctccgtcgctgtcatctgtcgccgtgacgtcgacttttttttgccgagtaccgagtggttgccgagtgtccgacaaaaaatactcggttaaagaccgttgccgatgtcagttcaccaagaccttttttatcaagtgtcatacttgataaagtcttccccaagtattttcaaaactttgtcgattacctcacacaccaaggggtaggatcgtgttgaggcggtttgctgcttgtttcttgctttcgccgaaccaacggaccatgagcgcataaaccaatgggcgagcggccgcatatacaggcctgcgcggcgagcctcaccttgctggatttgaatgccccctcggcatggtcaatgaccatgcatctttgtgcagaacatataaaatgcacaaattaattaattaataaaaatacatgaaagagtacataatttgtttgtggattattaattatatttataaatatattatagtatattatattttatctttatctaactacaaaaataattatagattgttaacttttttttgcattactggtaagattttgtcgattttataattttgtctaacatgttttattatcaatctaaaattgcaaattatatgttaacgaactaaatttgtaataaaatatatgtaataaaggtcgtctaaaagtaagctaaaattcacgcacctaaattttatttagtaaagtgttttttcttgacatatgacactagtgttttcaaagcttgtctcttaatttaggattaaattaataggcctctctaaattgctttttataagataaataaatcctattccgtctgctcatcttcaaagaacatttcaatgaaacaaatatttgggaaaaccaatggaaaaagaataaaaataaaaattgaaaaagagaagtggaggaaaagtagggttccattccacggcctgtcgagtgtcgacccacaaaccacgcaaccgatatattccccagcacccccagtctccagccgtccaaccgagacaccgcgtgcgaaccaagcagaccacaacaagaagaagcgtagtcgtcgccggaaggaaaggcgcggagcaagatctcgtggcaggcgtacgtcgacgagcacctgaagtgcgagatcgaaggacagcatctcagcgccgccgccatcgtcggtcacgacggcagcgtttgggcgcagtccgagagcttccccgaggtcattcactcccaccttatctcttcccgctctacttgcttagttgctttatccatgttcagtgctcctgcgctgatttagacccgcgcgactctggatctgccccagccgtgcggcccggccggatcggagcgaggggatgtccggatctcggtcgctgaggcgagatgcggatctaaggccccttccctcggtgacgtggttgctgctgctgctgttctccgggcgtttgatgccaatatagctgagatcaagcttgatgatctgcgtactgtggatttgctagtgagggatggccggatcgggctgttggcgtccgacctgatttggttgttcgcttgaatagacttacttaccagtggacagtggtagtgatcgttcagcagcagtagagcaatttgttttacatgtaaaatttgagtggctgctagagatgcaccaatggccgagctgcatgtgtttattcctatatagcgacagttgtgtgattgatccaaaacgcaaaattttagttttcatcaaatgatgatttcctgatgccactacctgtgtgtgaggcacttattaaatgaaaagtaactttcggttgggatctgggaatctgtattgtgttccgtggttggtcacagcttgtggcctgcattgttccataggccaaatggctaggcaaggaaaataaatcatgtaatttggaaaaaaataactgccatagtcagtagtgtacagcagtacatgcttgtccgggccttttatttcaccatcgtgttgtggtatatggagtacttggattgtgcgatgctttatacatttgtttgttctagcagagtttgagtttatatttctattcttatgtagggcgtcacgagtgtcgtcaattgttacatccttacatccttgtgttcacaatatgatccatcaactctttcttctgcggtctcttatccctgtgttctcatgtccatgtttcctgcttcttgttgcagttaaagccttaggaggtaaaccatggacaacaaccccaacatcaacgagtgcatcccctacaactgcctgagcaaccccgaggtggaggtgctgggcggcgagcgcatcgagaccggctacacccccatcgacatcagcctgagcctgacccagttcctgctgagcgagttcgtgcccggcgccggcttcgtgctgggcctggtggacatcatctggggcatcttcggccccagccagtgggacgccttcctggtgcagatcgagcagctgatcaaccagcgcatcgaggagttcgcccgcaaccaggccatcagccgcctggagggcctgagcaacctgtaccaaatctacgccgagagcttccgcgagtgggaggccgaccccaccaaccccgccctgcgcgaggagatgcgcatccagttcaacgacatgaacagcgccctgaccaccgccatccccctgttcgccgtgcagaactaccaggtgcccctgctgagcgtgtacgtgcaggccgccaacctgcacctgagcgtgctgcgcgacgtcagcgtgttcggccagcgctggggcttcgacgccgccaccatcaacagccgctacaacgacctgacccgcctgatcggcaactacaccgaccacgccgtgcgctggtacaacaccggcctggagcgcgtgtggggtccagacagccgcgactggatcaggtacaaccagttccgccgcgagctgaccctgaccgtgctggacatcgtgagcctgttccccaactacgacagccgcacctaccccatccgcaccgtgagccagctgacccgcgagatttacaccaaccccgtgctggagaacttcgacggcagcttccgcggcagcgcccagggcatcgagggcagcatccgcagcccccacctgatggacatcctgaacagcatcaccatctacaccgacgcccaccgcggcgagtactactggagcggccaccagatcatggccagccccgtcggcttcagcggccccgagttcaccttccccctgtacggcactatgggcaacgctgcacctcagcagcgcatcgtggcacagctgggccagggagtgtaccgcaccctgagcagcaccctgtaccgtcgacctttcaacatcggcatcaacaaccagcagctgagcgtgctggacggcaccgagttcgcctacggcaccagcagcaacctgcccagcgccgtgtaccgcaagagcggcaccgtggacagcctggacgagatcccccctcagaacaacaacgtgccacctcgacagggcttcagccaccgtctgagccacgtgagcatgttccgcagtggcttcagcaacagcagcgtgagcatcatccgtgcacctatgttcagctggattcaccgcagtgccgagttcaacaacatcatccccagcagccagatcacccagatccccctgaccaagagcaccaacctgggcagcggcaccagcgtggtgaagggccccggcttcaccggcggcgacatcctgcgccgcaccagccccggccagatcagcaccctgcgcgtgaacatcaccgcccccctgagccagcgctaccgcgtccgcatccgctacgccagcaccaccaacctgcagttccacaccagcatcgacggccgccccatcaaccagggcaacttcagcgccaccatgagcagcggcagcaacctgcagagcggcagcttccgcaccgtgggcttcaccacccccttcaacttcagcaacggcagcagcgtgttcaccctgagcgcccacgtgttcaacagcggcaacgaggtgtacatcgaccgcatcgagttcgtgcccgccgaggtgaccttcgaggccgagtacgacctggagagggctcagaaggccgtgaacgagctgttcaccagcagcaaccagatcggcctgaagaccgacgtgaccgactaccacatcgatcaggtgtaggagctcaagttcgtcatgttctgttttggtcatttgggcaccaaagtttgcgcctcatttggttctgtaatccgtgaggtcgtgcatgtacttggcgtattgcatgcagtgaataatttagcttgggtttgtttgttgggggcagtgttggggacggatttggattggggtttatgcttggcatcgcgtcgtatcgaaactcagctgctgtttcgctgagtaatgtacatttccctggtaatggtacttgtggactctgatgcttttatgggaacgagtgcattttactgccgtgcttggccgtgctgttgaattattcccgtagtgtattaaatattggatgcaccagattgtttgtcccatttacgcataattttcccgtcatatgttgaattatttacgaattataaaatatgtaatttatatttatttttatgaagtatataaaattttacttttttgttatataattaaatattttttgttttgataaaatggtatgttttattttaaataaaaatctataaaaaaagatgatatggtggctggaaggtaacgataaattatgtatactaaagtattaacgatagtaaatatagttggacgatagatgatgtgacggggtttgttcttttgattttaatctataatgcttgtctatagattacagccgcatcaatttaaatagctgcttttaatctgaagcgaacaaccgataaaaatagacattaggccatctccaacagttcgaccgtgcaaagtattattttgctattgcactatttgaataatggattttagaatagggagtgataaaaagtgagataaaaagtttaataaaaatagtcttagatccaacaaatatacgctgccctcccatcagttgcctggacccatgccaagaagtgccagccatagtcgtatgaaaaatgattctaagggcgttcgcaatggttgtcttaaatagttagctattgtgaagctgaaactaagagaagaataaaaatataatttatttcacggaccgcgatcgcttaattaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccccgatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggagctcgtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctgcaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgtctcctaatatatggttgattgttcggtttgttgctatgctattgggttctgattgctgctagttcttgctgaatccagaagttctcgtagtatagctcagattcatattatttatttgagtgataagtgatccaggttattactatgttagctaggttttttttacaaggataaattatctgtgatcataattcttatgaaagctttatgtttcctggaggcagtggcatgcaatgcatgacagcaacttgatcacaccagctgaggtagatacggtaacaaggttcttaaatctgttcaccaaatcattggagaacacacatacacattcttgccagtcttggttagagaaatttcatgacaaaatgccaaagctgtcttgactcttcacttttggccatgagtcgtgacttagtttggtttaatggaccggttctcctagcttgttctactcaaaactgttgttgatgcgaataagttgtgatggttgatctctggattttgttttgctctcaatagtggacgagattagatagcccggaaatttaccggtgcccgggcggccagcatggccgtatccgcaatgtgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccggggatctggctcgcggcggacgcacgacgccggggcgagaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgcccatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctagatttaggtcgtgaagatgggctcgagatcgttcgtaatctggcggcaaagtctgatattccaatcataattatcagtggcgaccgccttgaggagacggataaagttgttgcactcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaatta

SEQ ID NO: 22 - DNA, 16210 bp, synthetic construct
  misc_feature 1..16210: note = Recombinant vector
  source 1..16210: mol_type = other DNA
attcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgctgcaggaattggccgcagcggccatttaaatcaattgggcgcgccagctgcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaactcgagacttttcaacaaagggtaatatccggaaacctcctcggattccattgcccagctatctgtcactttattgtgaagatagtggaaaaggaaggtggctcctacaaatgccatcattgcgataaaggaaaggctatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgaacaatcccactatccttcggtaccggaccctctgcctttctgttcttcaaacgatgtctcatgtctgcgctggacaactttcttgttgccgcctgtcgcttgcgctgtgctgactggacgcagctccggaggtttggttgtgcttggttttcgtagagaactcgccacttgccgcccgcacgttcttggtgtttcctcctccccgctgtgttctgcgcacgggctttttctgagagacccatgtttcccttttacttttataaacagtatacatgctatgtttctagaaggaggggaaacctaatccccctaatccaatggcggggaggaaatagggtggggtggggtggggggagggaaatatctcgctactttttaatccggacaagctcatttgcgtttgcgtctgaatgatgatgactgcaatgctgatcgcacctcgggtgtcggatcaccagcttttggctgctctcaccaaatcagctgcaagaagattagagcacaaaagaattacagaaagagagcctttttcttttcttccttgtggggttcctttcatttcgtgctctcctttctctgccagccagtccgtccttgcgtccactgcacctgcacacaggtcaccccgacccgcactgttctagactccattagaaaaaaaaaggtctgaacctttccgaaaccagccagccattggtctggcaggccagcatatgctaattggatttttttgccgcatcattgagtgcgccatcaggatttggaaatcctggttttgagtaatacagtaatttggcattatccattgccgaattcccaagctccgtcagcttgaacgtggacccctaccatctgcaccagctcggcacctcacgctcgcagcgctaggagcctaggagcagctgcccgtctatttattggtccctctcccgtcccagagaaaccctccctccctcctccattggactgcttgctccctgttgaccattggggtatgcttgctgccttgctctcctgttcatctccgtgctaaacctctgtcctctgggtgggtttttgctgggattttgagctaatctgctggtcccggtagaaaaagatcatgtcccctgacgtgctcaagcgctcgccttagccgcgtccttgccccccgccattttttgcggtttcggtgtgttcccgtgactcgccgggtgcgtcatcgcctgaatcttgtctgggctctgctgacatgttcttggctagttgggtttatagattcctctgatctaaaccgtgcctgtgctgcgcacagaactctcccctgtcctttcctggggttttggttacgtggtggtagtaagcttggatttgcacatggataaagttgttctaagctccgtgggttgcttgagatcttgctgttattgcgtgccgtgctcactttttttgcaatccgaggaatgaatttgtcgtttactcgttttggtggattattagcgcgaaaaaaaaactcttttttttttgttcttttactacgaaaagcatcttcttggattttgctatcttcttttactacgaaaaactcttgagtctaggaatttgaatttgtgatgtccattcttgcagtgcgctgtgctttattgggaagccaaatcctattattttctgcctctagggtctgaatggaatcagtactcttgagacagaaaatcaatccaatcaagttgatttctttctttaaaaatattatcacagaactaagtgcttgtgcggaatcagtactggcttttgtttggtggaggatcaatacttgcttttgtttgggggtggcaactgttttgctataagattccatgtgttcctgttgagatgaatcatatatagtatagctgcatactacaaatctgtttttcaaatttaggttgctttggcatgatctatttttttgtcagacagactttctaagtggtagctcttgatttcttgttcttgtacaactggtgctgctgaatcttgaccgtatagctcgaattgcagtattctgaaccatcgagccaaggctgccaagctgactcgcctccacagtcttcgcgaacgccttggtgccaccttctcctcccatcccaatgaactgatagcactcttttccaggtgggcttaccaaaatcatataacttgcatttcattcggtactgaaagttgttaatttgttattctcttcatgcctgtcttaatagcacacccagatgtaaacacgagattatgcaacttcttacttggtttcttttgttggcaccatcatgcatgctaattgctaaggatgttacctattcatccttgactcatattatcatatgtaatgattttatgatcacgagactattgattgtgaagcatagtatagctgttcttcagtttttgtacccttttgtttttttccttaagctagaactggtacaatttagttgataagacagtgtagtttgtagtacgtcatttgacagattgtttgtctttagctggtaaagtgccatttaatatctgtatccttcagatctaataaaaaggatatgagatgtccatcacaagaggggaaaaattacatgatctgagatgtaacatccgtttttatttgtgaaataccacttctacaggtatcttcactagggtaaaccatggacaacaaccccaacatcaacgagtgcatcccctacaactgcctgagcaaccccgaggtggaggtgctgggcggcgagcgcatcgagaccggctacacccccatcgacatcagcctgagcctgacccagttcctgctgagcgagttcgtgcccggcgccggcttcgtgctgggcctggtggacatcatctggggcatcttcggccccagccagtgggacgccttcctggtgcagatcgagcagctgatcaaccagcgcatcgaggagttcgcccgcaaccaggccatcagccgcctggagggcctgagcaacctgtaccaaatctacgccgagagcttccgcgagtgggaggccgaccccaccaaccccgccctgcgcgaggagatgcgcatccagttcaacgacatgaacagcgccctgaccaccgccatccccctgttcgccgtgcagaactaccaggtgcccctgctgagcgtgtacgtgcaggccgccaacctgcacctgagcgtgctgcgcgacgtcagcgtgttcggccagcgctggggcttcgacgccgccaccatcaacagccgctacaacgacctgacccgcctgatcggcaactacaccgaccacgccgtgcgctggtacaacaccggcctggagcgcgtgtggggtccagacagccgcgactggatcaggtacaaccagttccgccgcgagctgaccctgaccgtgctggacatcgtgagcctgttccccaactacgacagccgcacctaccccatccgcaccgtgagccagctgacccgcgagatttacaccaaccccgtgctggagaacttcgacggcagcttccgcggcagcgcccagggcatcgagggcagcatccgcagcccccacctgatggacatcctgaacagcatcaccatctacaccgacgcccaccgcggcgagtactactggagcggccaccagatcatggccagccccgtcggcttcagcggccccgagttcaccttccccctgtacggcactatgggcaacgctgcacctcagcagcgcatcgtggcacagctgggccagggagtgtaccgcaccctgagcagcaccctgtaccgtcgacctttcaacatcggcatcaacaaccagcagctgagcgtgctggacggcaccgagttcgcctacggcaccagcagcaacctgcccagcgccgtgtaccgcaagagcggcaccgtggacagcctggacgagatcccccctcagaacaacaacgtgccacctcgacagggcttcagccaccgtctgagccacgtgagcatgttccgcagtggcttcagcaacagcagcgtgagcatcatccgtgcacctatgttcagctggattcaccgcagtgccgagttcaacaacatcatccccagcagccagatcacccagatccccctgaccaagagcaccaacctgggcagcggcaccagcgtggtgaagggccccggcttcaccggcggcgacatcctgcgccgcaccagccccggccagatcagcaccctgcgcgtgaacatcaccgcccccctgagccagcgctaccgcgtccgcatccgctacgccagcaccaccaacctgcagttccacaccagcatcgacggccgccccatcaaccagggcaacttcagcgccaccatgagcagcggcagcaacctgcagagcggcagcttccgcaccgtgggcttcaccacccccttcaacttcagcaacggcagcagcgtgttcaccctgagcgcccacgtgttcaacagcggcaacgaggtgtacatcgaccgcatcgagttcgtgcccgccgaggtgaccttcgaggccgagtacgacctggagagggctcagaaggccgtgaacgagctgttcaccagcagcaaccagatcggcctgaagaccgacgtgaccgactaccacatcgatcaggtgtaggagctctacggggaaagaaggagaagaagaagaagaagcccaggccggagaaccatcgcctgcatttcgatctgtttcaccgcaattcgcattgttagtcgtgtattggagttatgtgtacttggtttccaagaactttggttccttctcgtttttttttccttgcttgtttgagcgtttttgggcagcgctggcctggttcctagtatggtgggaattggctgcaccttttgcttcgaataaaaatgcctgctcgttcacctgtcttccagagtgcaatgcgatgttctgttgcccaggtcgtgtggttctgactgatggcgatgttgtgttcttctgttaatcgcctgttttaacgtggtaggctgatgcttgttcttgttgagaaagcttgctgtgccagacatggctgcttgaatacaagtgaaggaaaaaaaaagccatgccaagtaaagttgcacaaaatttcaactgctcagtggaccactggaccatgttcttggttattgcagttgcagggcttcacatggcgtttggacagcagtcttggattgatgcataaagaggtggtggttaatgaggacgcaaggccgttccctcagagtcagtcacaaggttgcagaggtcacggttctcttccctttccgcttcctgtcacatcggaattgttgtttacgccatctgcccatcacccaccaagtctatgtttctgtactggatctttcaatggcggaacgcgcttagttcttcgtcacagtcgaatcacatgatctaatcgatgtctttaatctcgctgtaaaaagggtgggacggtgggtgcagggtagggaccagggaaggcctgcctaaacgtatccataaacatgcacagcaaccctaagatattatactgcctacttcctaagatatagttatttctagtctattttttttccgtccacatccaaataagtgataatatatagacatacatatatatactatattcatcatagattaatgaacgaatgtatacttagtttaaacctaattatattttaggaaggatggagtatgaaacatgacaatacaacaaaaaaaaatcatgtaattgcatatcgtcaaagttatctgaagtaaccaatccagggggaaatcccgttagcaaacatacaagagcaccgccccactacatcccagaaaataaaacaaaaccagaactcagatggataaataatactaccggaccgcgatcgcttaattaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccccgatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggagctcgtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctgcaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgtctcctaatatatggttgattgttcggtttgttgctatgctattgggttctgattgctgctagttcttgctgaatccagaagttctcgtagtatagctcagattcatattatttatttgagtgataagtgatccaggttattactatgttagctaggttttttttacaaggataaattatctgtgatcataattcttatgaaagctttatgtttcctggaggcagtggcatgcaatgcatgacagcaacttgatcacaccagctgaggtagatacggtaacaaggttcttaaatctgttcaccaaatcattggagaacacacatacacattcttgccagtcttggttagagaaatttcatgacaaaatgccaaagctgtcttgactcttcacttttggccatgagtcgtgacttagtttggtttaatggaccggttctcctagcttgttctactcaaaactgttgttgatgcgaataagttgtgatggttgatctctggattttgttttgctctcaatagtggacgagattagatagcccggaaatttaccggtgcccgggcggccagcatggccgtatccgcaatgtgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccggggatctggctcgcggcggacgcacgacgccggggcgagaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgcccatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctagatttaggtcgtgaagatgggctcgagatcgttcgtaatctggcggcaaagtctgatattccaatcataattatcagtggcgaccgccttgaggagacggataaagttgttgcactcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaatta

SEQ ID NO: 23 - DNA, 15881 bp, synthetic construct
  misc_feature 1..15881: note = Recombinant vector
  source 1..15881: mol_type = other DNA
attcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgctgcaggaattggccgcagcggccatttaaatcaattgggcgcgccagctgcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaactcgagacttttcaacaaagggtaatatccggaaacctcctcggattccattgcccagctatctgtcactttattgtgaagatagtggaaaaggaaggtggctcctacaaatgccatcattgcgataaaggaaaggctatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgaacaatcccactatccttcggtaccggacccatgccttacgatcatctgacatcttaaatatttcaaactgcagtatttcaaaaactgtggttttgtcaaaaactttgttcccaaactgagactaaaatgtagtgctagataataaggccttacccgttggaatgacaaaaaagaaacagaaaacacctgaaaaatagtattgccttggctctaaattataaatcgttttggttttatttaatatctattttttaagcatactaaaatctatgtacctatgaaaaataaaatatgaactacaatttaagactgagtacagtgtacagcgataagttatatgaaccacaattgtttttgaaaggaaaaaaactaaattgacaagagaaacaatcaactcttttagaaactttgtataccaaaaaacgaaataaagaagaagaaaaaaatacacacgatgagtttattagcccacaaaacatgaaaaaaaatgagagttaaagcctaactcaggcccacaaagaatcacaagcgcccatattcacagccgccgaaaggtttcggcccccagttggatctcagagcccagtgtgaaataggactgacttgtctctgatggttacgttacgtagggattgaaacaaacaggcagtgagccgatgtggcagaccggagcgcagaagcgacgacgaccatgaactgaaggcgctggcagtgggccatgcgcccagaacgctgacgaaccacgacgacgcggacgttctacgaggtcggtggggccgtcggcagcccgccggagcatattcgccgtcgacagaggcagagaaggggaatgtgtgggagcatattccacccgtgggaagcctcggagtcggagatgtgcagatctgccggacgcacatgcgcacagctgccgcaatagaaaccagtgtctttttattttattttataagaaaaaactagaaaagaaaatccagtcttctatacaggcctgcacagagagagaggagggaaggtcaaatcgtagaaaaaaaatgcttccttttattgttacgacattggcctgaatatatactcattgtttctttccttaacttcaacaattattgattttgacaatccattttttattttagttcgccaaggttttggttggagaagaatttaaaacctagccaagcagtcgagcacgagagccgccgccgccgccgccgccgttggcaggacctgctgacctctgacgaccggacacggacgttttccaaagtccaaactcggggtaggtaggtcgtgaggctgctgcgttcgccaagggaagaagcatctagttgaagctagaaacgggcaggggggaggggccgacgctatcggaaatctgaagcccagcacggacggacggacgggcaggggcggcggcacatcctcgtctgaccgccgcgccgcgtccgacatgcgcaagctgctgctcttcccgcgcccccacggcggccgggccggcagagcacgggacgcgcggggcgggcctgacgtcgttttcacgtggtccgtcaccgttgcttgcatagcgtacaggagtatagtcccatttccgaaaccaaaaaaagtaaacaaaaaaatgccgtcgcattagttggctggcaagggaagaccaagatggttttcaaatcaaacatgtaaaaaaatgtttctttcttttttgaacgaacttaccagttcgaattctttaccggcattattggtttaaattgttccccaagaaagaaagaaaagaaaagcggggggaaatatgccttccttccttcttgcgtcacagaccacgcgcggatacaccgcacaacagccaggcagcaagcagagcacccaccgccggaagcggccgtcggacagaacgacacgtggggcacggcacagtccgggcccgcacgtcatcgaagacacctgacctgcccatgcgtcttctagagaaaggcggacggacaggtcacccccgcacctcattccaggagttgcatttcgctttctcctttatttatttataccaataaaaaataaatccgccttttctcctccctatcgtgtgtcttcctctcgccggctttaaaaacgcacacaagcgctaaaaccctctccaccgtccacctcagctcccatatccgctcccctacctctccagcatcctcccgtctccgtcgtctcgtctccgctcctcacctcgccgctagccaagggtaactcctcgctcccggccggcccccgcgtcggggttttccatttactactactcgctgctccctcctgctccgtgctcagactcagatccgaccaaagcggtttcgctgactaaattctccttcgtttttttttctttttctggacggattcctggacgcaggcaagatcaagatcggaatcaacggtgagtctgtacccccaacacaaactcgttcttcctgctcggttcgttgggtctggactctggagtgatctgagtggggtctctgcgcgcctgcgtgctcaggtttctgaaggatcggcagtaaaccatggacaacaaccccaacatcaacgagtgcatcccctacaactgcctgagcaaccccgaggtggaggtgctgggcggcgagcgcatcgagaccggctacacccccatcgacatcagcctgagcctgacccagttcctgctgagcgagttcgtgcccggcgccggcttcgtgctgggcctggtggacatcatctggggcatcttcggccccagccagtgggacgccttcctggtgcagatcgagcagctgatcaaccagcgcatcgaggagttcgcccgcaaccaggccatcagccgcctggagggcctgagcaacctgtaccaaatctacgccgagagcttccgcgagtgggaggccgaccccaccaaccccgccctgcgcgaggagatgcgcatccagttcaacgacatgaacagcgccctgaccaccgccatccccctgttcgccgtgcagaactaccaggtgcccctgctgagcgtgtacgtgcaggccgccaacctgcacctgagcgtgctgcgcgacgtcagcgtgttcggccagcgctggggcttcgacgccgccaccatcaacagccgctacaacgacctgacccgcctgatcggcaactacaccgaccacgccgtgcgctggtacaacaccggcctggagcgcgtgtggggtccagacagccgcgactggatcaggtacaaccagttccgccgcgagctgaccctgaccgtgctggacatcgtgagcctgttccccaactacgacagccgcacctaccccatccgcaccgtgagccagctgacccgcgagatttacaccaaccccgtgctggagaacttcgacggcagcttccgcggcagcgcccagggcatcgagggcagcatccgcagcccccacctgatggacatcctgaacagcatcaccatctacaccgacgcccaccgcggcgagtactactggagcggccaccagatcatggccagccccgtcggcttcagcggccccgagttcaccttccccctgtacggcactatgggcaacgctgcacctcagcagcgcatcgtggcacagctgggccagggagtgtaccgcaccctgagcagcaccctgtaccgtcgacctttcaacatcggcatcaacaaccagcagctgagcgtgctggacggcaccgagttcgcctacggcaccagcagcaacctgcccagcgccgtgtaccgcaagagcggcaccgtggacagcctggacgagatcccccctcagaacaacaacgtgccacctcgacagggcttcagccaccgtctgagccacgtgagcatgttccgcagtggcttcagcaacagcagcgtgagcatcatccgtgcacctatgttcagctggattcaccgcagtgccgagttcaacaacatcatccccagcagccagatcacccagatccccctgaccaagagcaccaacctgggcagcggcaccagcgtggtgaagggccccggcttcaccggcggcgacatcctgcgccgcaccagccccggccagatcagcaccctgcgcgtgaacatcaccgcccccctgagccagcgctaccgcgtccgcatccgctacgccagcaccaccaacctgcagttccacaccagcatcgacggccgccccatcaaccagggcaacttcagcgccaccatgagcagcggcagcaacctgcagagcggcagcttccgcaccgtgggcttcaccacccccttcaacttcagcaacggcagcagcgtgttcaccctgagcgcccacgtgttcaacagcggcaacgaggtgtacatcgaccgcatcgagttcgtgcccgccgaggtgaccttcgaggccgagtacgacctggagagggctcagaaggccgtgaacgagctgttcaccagcagcaaccagatcggcctgaagaccgacgtgaccgactaccacatcgatcaggtgtaggagctcagagagatatttctgcctccctatcgtgggtcgtccccaatggcctttggtcgcagaccatctttgctgcttgtctatgctgagaataaatgtgaacggtgcccctggacgctggatccatgctggttttggacacggttgtctttttgtgtttaacttatctgctaccgtcctgtaacgaattcactaagttctagttcttttgtgctttgttaagaatataaacaatgaggtttcaattttggtggcgccatccggtctgattgcgtactagtatcatgcatatctgagctggtctttccggagtaacagtttttttttgtttcgtgtcaatgattctcagcctgttcatttggtcttaatccatatcggttactactataatgttgtcttgtctctatagattgcagcagtctaacacatgttcttggtatccttttgggtgtgtttaatgctatttgctctgtgttgtctttttcgttaatcgggacagagttttctctctgttttgacagtttcggtggttcttttttgtgtgtggtcagcgagtcacgaactgctggttggccagcgaaatagcgcagactattatgggtcttctgctggtgaatatggaaggacgaggttaaattttctgctttgatgtgtctgccctctgccgctgtgctgcatgcatgtgtttgggagcgaagcagcgtgctgtttgggcgtatggttggcatggtgttaaattatttcgctgattcagctttgggttatcactggttttgatggtcacgctgtgtcgtaagagagatcgtggcagcaaaatggagaaaaaggcagtgaaacaaactttcagggtgtggaatgtatgggattcgtatggttgaggccaggatttgtcaattggtcaagttgaaaagtaaaggcgagacagattggcgaggaccgaggagagattggggttcagaaacttcggttacactttaatcccctccatctccctccactcctcccttctacggaccgcgatcgcttaattaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccccgatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggagctcgtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctgcaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgtctcctaatatatggttgattgttcggtttgttgctatgctattgggttctgattgctgctagttcttgctgaatccagaagttctcgtagtatagctcagattcatattatttatttgagtgataagtgatccaggttattactatgttagctaggttttttttacaaggataaattatctgtgatcataattcttatgaaagctttatgtttcctggaggcagtggcatgcaatgcatgacagcaacttgatcacaccagctgaggtagatacggtaacaaggttcttaaatctgttcaccaaatcattggagaacacacatacacattcttgccagtcttggttagagaaatttcatgacaaaatgccaaagctgtcttgactcttcacttttggccatgagtcgtgacttagtttggtttaatggaccggttctcctagcttgttctactcaaaactgttgttgatgcgaataagttgtgatggttgatctctggattttgttttgctctcaatagtggacgagattagatagcccggaaatttaccggtgcccgggcggccagcatggccgtatccgcaatgtgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccggggatctggctcgcggcggacgcacgacgccggggcgagaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgcccatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctagatttaggtcgtgaagatgggctcgagatcgttcgtaatctggcggcaaagtctgatattccaatcataattatcagtggcgaccgccttgaggagacggataaagttgttgcactcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaatta

SEQ ID NO: 24 - DNA, 15436 bp, synthetic construct
  misc_feature 1..15436: note = Recombinant vector
  source 1..15436: mol_type = other DNA
attcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgctgcaggaattggccgcagcggccatttaaatcaattgggcgcgccagctgcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaactcgagacttttcaacaaagggtaatatccggaaacctcctcggattccattgcccagctatctgtcactttattgtgaagatagtggaaaaggaaggtggctcctacaaatgccatcattgcgataaaggaaaggctatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgaacaatcccactatccttcggtaccggacccaaatgaattaatatattatctaaattcagacgggaaaaagagagaaggaatcaggaaggctgagtgcaatattagtttttcattgagcacaatcttattatgtttgatgaacatcttgaacaaagtatgagtggaaaacaacacttatgttatatctgcagcacagattgttagaagtgaaggctaccttgagatggtgataggtctctgaacccatctgcattgcttcttcgaagttcatagcaccaacaagaagaatcataatttcctacaaataagaaatgtttatagttactccaaaactacagttaaacatattgatatggttgatatacatgaacaactaactgtcttaatatgtttctgtttaggcaaaagatgacacaagtggacaaagtaagggaataatgcattcaacttctcagactctaaaggtttggatgaatacttgaatgggaagaccatttccagcatgctttccaccattaatgactgtatttgcagggacaggaagagttgtagcgcttttgccaacaagatccgctatatgcttgtagagtggaacctgcacagcaacaagcatccaatgatctgagaataagaattatgtgcggtggcatgacacatttaccaaattatgatgattttagcagaagagcagcacctctttttcagcagcaccagctttgcaagctgcaattgacactgccagcatagtatttgctccaagctcagcctgcatttcgcacaatatcatatctatttccatttggataaaatctatttgccagaaatatgggaacggaatcaagatacagaaaaaagttgaggctgtagtggtaagtgggaacacaagtacagtgaaaagatagcattattttccgaacagtcaaattaaccgaaataagtagtatcacatactgaccctaaattgactacacccaatgtaaagaattgcttgtgcatattgagccatcagttgtttgtttgtgtaatggatttgacaatgcagaattcaggagcaaaaaaaaccgcaaaggagagagagggaaaggatctccgagtatcccacctgtggcgcgccttgtcaagtccatgatggcctggtcgatctgcgcctgctgctgcggatccatcccaacgagtgcctccgacaccttgtcgttgatcacccgcaccgcgtaggcgacccccctggcgaggaacttccgcctctcggaggccccggccgcgtcggcggcggcgcccacgagcgcgcccacgctagccgtagatgctcggtgcacggccttgttggtgtgcaactcgacctcaacggccggtgcgctacgcccgtcaaggatctggcgcgcccgcaccctcgtgatcacggtgggcgcctcccgcctcatgtggccgacgatgaaaagcgccgggtcaggggccttggcgcggagcgccgtattcagggcctcctcgctcttgcgcgagagcaggtgcttccccaggtactcctgcactgacatggctgccacagcggtggaattttcttgtcctcacgacttcgccacgtcgtcctcacctccctggcttcccacgactccgccgtatcgaggtgctcggtgggtcgccttaatccgaagtccgaactaggaagacgagacagagaggtctgaggaatgggcctcgtttgatgttgagctgaattatttttccagcataagcccaggtcttggtccatgaacaaaattactagaaacccagcccagtactacgatctaaaagagggactgccactggatagctctctctagcattctccacgctccaatacagcggcgtagggtctatccgggtctatccgcgaacacgtgagaactctccagaaactgctttctcctccacttcatctctctcgctttccctctataaaaagaccccttctaggaattgagggagacagcaagcagcgatccgaagctcaatcaattcactcaaacctcttccccaaatcttcgattagattctcgttgacaagaagactataaccgaacctgaccgtaaaccatggacaacaaccccaacatcaacgagtgcatcccctacaactgcctgagcaaccccgaggtggaggtgctgggcggcgagcgcatcgagaccggctacacccccatcgacatcagcctgagcctgacccagttcctgctgagcgagttcgtgcccggcgccggcttcgtgctgggcctggtggacatcatctggggcatcttcggccccagccagtgggacgccttcctggtgcagatcgagcagctgatcaaccagcgcatcgaggagttcgcccgcaaccaggccatcagccgcctggagggcctgagcaacctgtaccaaatctacgccgagagcttccgcgagtgggaggccgaccccaccaaccccgccctgcgcgaggagatgcgcatccagttcaacgacatgaacagcgccctgaccaccgccatccccctgttcgccgtgcagaactaccaggtgcccctgctgagcgtgtacgtgcaggccgccaacctgcacctgagcgtgctgcgcgacgtcagcgtgttcggccagcgctggggcttcgacgccgccaccatcaacagccgctacaacgacctgacccgcctgatcggcaactacaccgaccacgccgtgcgctggtacaacaccggcctggagcgcgtgtggggtccagacagccgcgactggatcaggtacaaccagttccgccgcgagctgaccctgaccgtgctggacatcgtgagcctgttccccaactacgacagccgcacctaccccatccgcaccgtgagccagctgacccgcgagatttacaccaaccccgtgctggagaacttcgacggcagcttccgcggcagcgcccagggcatcgagggcagcatccgcagcccccacctgatggacatcctgaacagcatcaccatctacaccgacgcccaccgcggcgagtactactggagcggccaccagatcatggccagccccgtcggcttcagcggccccgagttcaccttccccctgtacggcactatgggcaacgctgcacctcagcagcgcatcgtggcacagctgggccagggagtgtaccgcaccctgagcagcaccctgtaccgtcgacctttcaacatcggcatcaacaaccagcagctgagcgtgctggacggcaccgagttcgcctacggcaccagcagcaacctgcccagcgccgtgtaccgcaagagcggcaccgtggacagcctggacgagatcccccctcagaacaacaacgtgccacctcgacagggcttcagccaccgtctgagccacgtgagcatgttccgcagtggcttcagcaacagcagcgtgagcatcatccgtgcacctatgttcagctggattcaccgcagtgccgagttcaacaacatcatccccagcagccagatcacccagatccccctgaccaagagcaccaacctgggcagcggcaccagcgtggtgaagggccccggcttcaccggcggcgacatcctgcgccgcaccagccccggccagatcagcaccctgcgcgtgaacatcaccgcccccctgagccagcgctaccgcgtccgcatccgctacgccagcaccaccaacctgcagttccacaccagcatcgacggccgccccatcaaccagggcaacttcagcgccaccatgagcagcggcagcaacctgcagagcggcagcttccgcaccgtgggcttcaccacccccttcaacttcagcaacggcagcagcgtgttcaccctgagcgcccacgtgttcaacagcggcaacgaggtgtacatcgaccgcatcgagttcgtgcccgccgaggtgaccttcgaggccgagtacgacctggagagggctcagaaggccgtgaacgagctgttcaccagcagcaaccagatcggcctgaagaccgacgtgaccgactaccacatcgatcaggtgtaggagctcacctcggtctgcggtcgtcgtacctgcgtggtttgaggaacggcagttcgcctcggtcgttctgtgaaataaaattgggttacaagaattatggcgtttgtcaatatggtcgtaatgtcgtaggatggtggaatgtggtcacaaactttgcgtatgttgggtctactggtggtgtctgaatctatgtatggatgtcatgagtttgtctactagagttctagttcctgtggtgttcgtatgatgtatatcatgagttaactctatctaaaatttcttcattatggtatttttaagaaataaattctttattatggtttttttaaagggactatgtccatcctaacaagagcaacgtccaataattctctaaaacttaaatttaagaaccgaataaaaaatgagtagtttttaaatactttctatctaaccttatttactcttctccatattttagtagttttttaaatagacttactaaatttagttactctatattttggtaactcgacagaatacaatctgtgattaatttttctcacatgtggtagctagatacgacacttttttttttactttttagcacatgcacaatggagtagttagattcaacacttatgacacaagttttttcttcaccgttggactattattgtcgcatatgttgccaccgcgaaccaccgctcctctataggttatgtcaatcgacctttcctcttttgtcttcgacatcatccaagactaacaaagttagattcaaatcaaactagatgtccagactggcaaagttagatttaaaatcaaactactatctacatataagcaactattagggactaagttatttttctacttcaatagttgttgctagcaacttgctaaacataattttagaaaacttttttagagaactattagagttgctctaaaaggttttgtagtcccatttactgttttgtgactattattaggtggagtttgggcagtaaaaaggccaaatagaaaatggggttgagattgggaaaacggaccgcgatcgcttaattaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccccgatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggagctcgtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctgcaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgtctcctaatatatggttgattgttcggtttgttgctatgctattgggttctgattgctgctagttcttgctgaatccagaagttctcgtagtatagctcagattcatattatttatttgagtgataagtgatccaggttattactatgttagctaggttttttttacaaggataaattatctgtgatcataattcttatgaaagctttatgtttcctggaggcagtggcatgcaatgcatgacagcaacttgatcacaccagctgaggtagatacggtaacaaggttcttaaatctgttcaccaaatcattggagaacacacatacacattcttgccagtcttggttagagaaatttcatgacaaaatgccaaagctgtcttgactcttcacttttggccatgagtcgtgacttagtttggtttaatggaccggttctcctagcttgttctactcaaaactgttgttgatgcgaataagttgtgatggttgatctctggattttgttttgctctcaatagtggacgagattagatagcccggaaatttaccggtgcccgggcggccagcatggccgtatccgcaatgtgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccggggatctggctcgcggcggacgcacgacgccggggcgagaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgcccatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctagatttaggtcgtgaagatgggctcgagatcgttcgtaatctggcggcaaagtctgatattccaatcataattatcagtggcgaccgccttgaggagacggataaagttgttgcactcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaatta

SEQ ID NO: 25 - DNA, 15884 bp, synthetic construct
  misc_feature 1..15884: note = Recombinant vector
  source 1..15884: mol_type = other DNA
attcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgctgcaggaattggccgcagcggccatttaaatcaattgggcgcgccagctgcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaactcgagacttttcaacaaagggtaatatccggaaacctcctcggattccattgcccagctatctgtcactttattgtgaagatagtggaaaaggaaggtggctcctacaaatgccatcattgcgataaaggaaaggctatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgaacaatcccactatccttcggtaccggaccctagtcaccacaccatgatcatcttgtttgttgtagaatacccacttgttacctacaacaatttggtttggacgtggaactaaatgcaagaccttattcctcgtgaagttgttgagcacctcttgcattgccaccacccagtctggatctctaagtgcatcctctatcatgtatggctcaatagaagacacacaagagtaatgttcacaaaaatgagcaacttgagagcgagtgattaaccccttttgaatgtcaccaaggatggagttgacggggtgatatcgctgaattgcttggtgaactcttgagtgaggtggtctttgatcttgaatctcttggtcatcctccttttcttgaacaacttcatctcccccttgattgattcgttcttcttgaggtggctcattgtcttgaacttgatcttcttcttcttcttgagctggttcctcatcttaagttggtggagatgtttgtatggaatatgatagttgatcatgtgcttgtgaaggctcttcgggtttttgtggacacatgtctccaatggacatgtttcttagcgcgatgcatggagcctctttatcatctaaatcatcaagatcaacttgctctagttgagagcctctagtggacacacatcttcgggttaaatggttttatggaatattttttcttcacagatacaaatagtatcgaatatttcagatatttcatggttttgtcgaatacaaatataaaatcggatagagaaaactaaatttaattatatccatttccatccatattaaaattgaatacggatatagatatccatattagcattttatttgaatacgaatatatataatttggatgtctagacattcgaatccatctctaattagtgtgggaatgagggacactgaaaaacaatgacgtgcatggtgacatcatacaatagtacaattctgacgacgatgaaggaatttacgtgcggatcagcgacaccacctggtttggtggttcctgtcgccggaggcgcaagaaataaagagggcacataactacgtgaaatcaagcccaattagtctgccttggctcccttatgctgttttaaaaagtttagggttaagtcattagtagactgttgcgggtttagaaatttttagagaattttgtttacaacagcccctaaactaaagtttttgggaacaaattttagtagtcttttaagttgctctaagactatatttttttagttgaggaggacagtgacaattttggagttgctctaaaaccatgttttttagtcgagtgggacagtggcaattacttaactacaatgcacaacaccaggaatccaggatgaaaaattactacaccgagggctagtttgggaacctcattttcccaagagattttcattttcccaaagaaaattagtttatttttccttgggaaaatagaaatcctttggaaaattggagtttccaaactagccttgattttttttcctaagatatgtgcagatctttctttgagaggacacaaaaaaaatggattgggattgggctcatcgaaggccgaatattcctatccatcgttcgtgccggattaggcccagaaccagaaaagagctaggccgggctgcagtagcactatcctctgtttcacaatattatgcacttttgatcactttatttatgtcaaaaatacttgatacatcacgttttattttcactttcgcctctataaaagtattaagggatttctagacaattcaaaatgtaattttataagactatgtttgtcattttataaaaaaaaatagtttgattattttggtgaacgtgccttggtcaaaatttgtgggaacggaggatgctatcaaattcgtctgcgcagatgtacgcccagtaacgaagtatcgtcaatcgactgatgaccccgtcaccgtcagcaagacagcaactcaacattcaaattcgaccgtaataacatccacatacatacacggagtatcaatctagactagaggagacgggtgaacgtggtgagcctccgcccataatgcaaccctactagtgctagctttcggccgcgaaaaaacgtccccacccccacgtctcaactttatagccgccccctccccaccgccgcggccgccacgcgcagcagcaacccggtagcaggagcgcagccagcaagctcaggcccccagccctactgccaccgcgccgaacgacaaggccgagccggcggagcaccgtccagctgaggaggaggaggccgcggcggccggcgaggatgaggacaccggcgcccaggtcgcgcccatcgtgaagctggaggaggtcgccgttaccactggagaggaggacgaggacgcgctcctggacttgtgaggcatccgcggccgcttcgattcccccccctccccgatccgatttgcccatgtcttgttgatctgatgtgcggcggctgtgcaggaaggcgaagctctaccggtaaaccatggacaacaaccccaacatcaacgagtgcatcccctacaactgcctgagcaaccccgaggtggaggtgctgggcggcgagcgcatcgagaccggctacacccccatcgacatcagcctgagcctgacccagttcctgctgagcgagttcgtgcccggcgccggcttcgtgctgggcctggtggacatcatctggggcatcttcggccccagccagtgggacgccttcctggtgcagatcgagcagctgatcaaccagcgcatcgaggagttcgcccgcaaccaggccatcagccgcctggagggcctgagcaacctgtaccaaatctacgccgagagcttccgcgagtgggaggccgaccccaccaaccccgccctgcgcgaggagatgcgcatccagttcaacgacatgaacagcgccctgaccaccgccatccccctgttcgccgtgcagaactaccaggtgcccctgctgagcgtgtacgtgcaggccgccaacctgcacctgagcgtgctgcgcgacgtcagcgtgttcggccagcgctggggcttcgacgccgccaccatcaacagccgctacaacgacctgacccgcctgatcggcaactacaccgaccacgccgtgcgctggtacaacaccggcctggagcgcgtgtggggtccagacagccgcgactggatcaggtacaaccagttccgccgcgagctgaccctgaccgtgctggacatcgtgagcctgttccccaactacgacagccgcacctaccccatccgcaccgtgagccagctgacccgcgagatttacaccaaccccgtgctggagaacttcgacggcagcttccgcggcagcgcccagggcatcgagggcagcatccgcagcccccacctgatggacatcctgaacagcatcaccatctacaccgacgcccaccgcggcgagtactactggagcggccaccagatcatggccagccccgtcggcttcagcggccccgagttcaccttccccctgtacggcactatgggcaacgctgcacctcagcagcgcatcgtggcacagctgggccagggagtgtaccgcaccctgagcagcaccctgtaccgtcgacctttcaacatcggcatcaacaaccagcagctgagcgtgctggacggcaccgagttcgcctacggcaccagcagcaacctgcccagcgccgtgtaccgcaagagcggcaccgtggacagcctggacgagatcccccctcagaacaacaacgtgccacctcgacagggcttcagccaccgtctgagccacgtgagcatgttccgcagtggcttcagcaacagcagcgtgagcatcatccgtgcacctatgttcagctggattcaccgcagtgccgagttcaacaacatcatccccagcagccagatcacccagatccccctgaccaagagcaccaacctgggcagcggcaccagcgtggtgaagggccccggcttcaccggcggcgacatcctgcgccgcaccagccccggccagatcagcaccctgcgcgtgaacatcaccgcccccctgagccagcgctaccgcgtccgcatccgctacgccagcaccaccaacctgcagttccacaccagcatcgacggccgccccatcaaccagggcaacttcagcgccaccatgagcagcggcagcaacctgcagagcggcagcttccgcaccgtgggcttcaccacccccttcaacttcagcaacggcagcagcgtgttcaccctgagcgcccacgtgttcaacagcggcaacgaggtgtacatcgaccgcatcgagttcgtgcccgccgaggtgaccttcgaggccgagtacgacctggagagggctcagaaggccgtgaacgagctgttcaccagcagcaaccagatcggcctgaagaccgacgtgaccgactaccacatcgatcaggtgtaggagctcaggttgaaggcaaacaagggtcaaatggatgccattccattcatttcgtttccaaggttcagcttccccgcaaattttcattgtgttttctccgagatgaatgtttgtgttcggtgaaatcagagtcgtcagtcatctacatagcttttcttggttgatagactgttattttaagtcgcatgtttatctgggatggctggggtcagcatgtttgtacaattatttggagttgcttttggaatggtcgcggtttgatgagttgcctacagccatgagatgtcttttcgctccacttttatggttcattcgttctcaataatatgggatgctatacttgtgttgatccattatcttgatgcatcgttgtctgtgcactgcaacaacaatacccatctgaacacccctatcaataaaataccattttttttctttccatcccactaatctaggcccactttctcactcttttcttatccactatattgtcaatatagaatctggggagagagagagagagagagagattgagagagagagagagagagagatttaggctccctcccttcctatattcaaaataggtatcgcctttgggtcacctgttggaatgatttattttagtatcgtccatatttaatttaaacaaatgttgaattttacatgcatattgatttaaagtttgttagtttatgagcatcaccaactaagatctctaatgccaaagagcatctccactagttccaaaaaactctctaaatttaatttaggatgttagtaaccagaaattatgctccaacagtttcctaaatgagttccttaaatataccaactttttaaatatctctatttagtcaaacttgagaaattgtttcacactcccaatagttgtcatcaatcacatcgtaaaatcatttgagttcgcacccgtgtacaagtggtgcactttaaataaattaaattacgaaaatgataaatttactatcgagttagcataatttaaaaatatataacaacataagaactgggaacggaccgcgatcgcttaattaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccccgatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggagctcgtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctgcaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgtctcctaatatatggttgattgttcggtttgttgctatgctattgggttctgattgctgctagttcttgctgaatccagaagttctcgtagtatagctcagattcatattatttatttgagtgataagtgatccaggttattactatgttagctaggttttttttacaaggataaattatctgtgatcataattcttatgaaagctttatgtttcctggaggcagtggcatgcaatgcatgacagcaacttgatcacaccagctgaggtagatacggtaacaaggttcttaaatctgttcaccaaatcattggagaacacacatacacattcttgccagtcttggttagagaaatttcatgacaaaatgccaaagctgtcttgactcttcacttttggccatgagtcgtgacttagtttggtttaatggaccggttctcctagcttgttctactcaaaactgttgttgatgcgaataagttgtgatggttgatctctggattttgttttgctctcaatagtggacgagattagatagcccggaaatttaccggtgcccgggcggccagcatggccgtatccgcaatgtgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccggggatctggctcgcggcggacgcacgacgccggggcgagaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgcccatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctagatttaggtcgtgaagatgggctcgagatcgttcgtaatctggcggcaaagtctgatattccaatcataattatcagtggcgaccgccttgaggagacggataaagttgttgcactcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaatta

SEQ ID NO: 26 - DNA, 15689 bp, synthetic construct
  misc_feature 1..15689: note = Recombinant vector
  source 1..15689: mol_type = other DNA
attcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgctgcaggaattggccgcagcggccatttaaatcaattgggcgcgccagctgcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaactcgagacttttcaacaaagggtaatatccggaaacctcctcggattccattgcccagctatctgtcactttattgtgaagatagtggaaaaggaaggtggctcctacaaatgccatcattgcgataaaggaaaggctatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgaacaatcccactatccttcggtaccggaccctttgtattgttgtatgtaatgaataatctttatatatatatatatatatatatatatatatatatatatatatatatatatatatatgtgtgtgtgtgggggggtgtgggggtgtgtgtattgatgtctcttggacaacaagattacacaaaacacaattagaataaaaaaatatcctcgtgtatataaacttgtccgtataccatattagaacacataaattttaggcaacattttttccatcaacattcttcaatcatcaaccaaaatttacggatacacaagataagagggggtatgtaaggttgtacgtaatgggctacatgataacatcaaagattatgcaagcaaatctcaatccaccaggcgatcataaacatcatagttcacatgcttcagttttaaagccatcgtagttcccgtgcaaagacaaaaacattagaattattcaatacaagttgcacaagatagttcaataaaatttaaaccacaatagtattatccaacaaagctagttcataccattatagtttctagtaaacaagaatagagaacatatattaagcaaacagaccacataggataaggctaaggatgtatttttgtctattgttttctgtgacatcgatctcgtttgcacgagtaaaactaaaacatgagaaaattccgataaaaaataggaatctagaaatacgaacggaaaacactaaaccatttttattcctgtttctaaaatatatcatctcgtttctgtttttctctgtaaatatgaaaacgatcagatcatagttaaaacggaatacagtaccggacgaaacaatatttctctctcgtttcccccttatagcatctccaaaagctccccagaagtctcccctaaatctatttttttggaaaaacacaaaaacatgtctccaacagttcctctaaagcgcccccaactttttcatagcccttaaaactccctcatttgtagctacaaatgaggggttttttgggctccccagaaacaaactgctgctttaagatgtttggttgaggagccaagtagaatggagtcgtttcatccctgattctaggaacggagccgttctgttctgtgtttggtaatctggaacggagcggctctgttttttgtttggttgcagagtgaacggaacggagcgtgactgtgagagcgggatgagaacggagcggctccgttcggttgattttttggagcggaatggttccggatttgaggagaatattccataattggagtcattccgttctagttcctttataaccaaacagcaacaaaactgggatagaatggttccgttctacttggctcttcaaccaaacactaactaagggacctgttggagaaatgattaaaatttaccctcacttattatttagatattccttaaaactaattttgagaagtcgttttatggagtgctcttggagatgctcttagttcgtagatctgattgtgtgtatgtatgtaagcatatgcgtctgtactacgatccacggtaaaaaagtcacaaacatataataatagcgtcattcaataaattgataacgtaaagtataaatgaacttttaactctgtttgagtaaacaactcgtgcagagactgcaatgaaatctattattttttctaaaataattatacaagttgaggaaatgctatttgttccactaagcgacgatgtattttgttttttaaaaaaatcgacgaggtactgctcggttattattttcacatgcaccgcgcgttgtttttgggccggcccatttgtattgcgaatttgcggagacgaatatgaccgaatggagtttagaaagcccagctcacttaggattgtctattttctcaagaaaagagagaatgggcccaaaggcctaaacaccaaaacccgatccgcttatgggatgtcatggacatggagtctgggaccgtccggcagatgagacgacagccgtcggatcagaaaccctagcgcgggaggctctccctattaatacccaccctgcaccccgcgggaggagtctttcctagggtttcgtagcttctagccgccgccgcgtccgcctcgccaagcgcagcagccgccgcagcacatcgctctctcgatctcagccatagcggaggtggagcagcaacaggacacgccgaagctcttcaaccgctggaccttcgatgatgtccaggtacgcgaacgagtcttcgcatctctgcatgcttcgattctttagccttgccgctactagcagtggatggaaccgacgatgaaatctgcaggtgaacgacatctcgctgtaaaccatggacaacaaccccaacatcaacgagtgcatcccctacaactgcctgagcaaccccgaggtggaggtgctgggcggcgagcgcatcgagaccggctacacccccatcgacatcagcctgagcctgacccagttcctgctgagcgagttcgtgcccggcgccggcttcgtgctgggcctggtggacatcatctggggcatcttcggccccagccagtgggacgccttcctggtgcagatcgagcagctgatcaaccagcgcatcgaggagttcgcccgcaaccaggccatcagccgcctggagggcctgagcaacctgtaccaaatctacgccgagagcttccgcgagtgggaggccgaccccaccaaccccgccctgcgcgaggagatgcgcatccagttcaacgacatgaacagcgccctgaccaccgccatccccctgttcgccgtgcagaactaccaggtgcccctgctgagcgtgtacgtgcaggccgccaacctgcacctgagcgtgctgcgcgacgtcagcgtgttcggccagcgctggggcttcgacgccgccaccatcaacagccgctacaacgacctgacccgcctgatcggcaactacaccgaccacgccgtgcgctggtacaacaccggcctggagcgcgtgtggggtccagacagccgcgactggatcaggtacaaccagttccgccgcgagctgaccctgaccgtgctggacatcgtgagcctgttccccaactacgacagccgcacctaccccatccgcaccgtgagccagctgacccgcgagatttacaccaaccccgtgctggagaacttcgacggcagcttccgcggcagcgcccagggcatcgagggcagcatccgcagcccccacctgatggacatcctgaacagcatcaccatctacaccgacgcccaccgcggcgagtactactggagcggccaccagatcatggccagccccgtcggcttcagcggccccgagttcaccttccccctgtacggcactatgggcaacgctgcacctcagcagcgcatcgtggcacagctgggccagggagtgtaccgcaccctgagcagcaccctgtaccgtcgacctttcaacatcggcatcaacaaccagcagctgagcgtgctggacggcaccgagttcgcctacggcaccagcagcaacctgcccagcgccgtgtaccgcaagagcggcaccgtggacagcctggacgagatcccccctcagaacaacaacgtgccacctcgacagggcttcagccaccgtctgagccacgtgagcatgttccgcagtggcttcagcaacagcagcgtgagcatcatccgtgcacctatgttcagctggattcaccgcagtgccgagttcaacaacatcatccccagcagccagatcacccagatccccctgaccaagagcaccaacctgggcagcggcaccagcgtggtgaagggccccggcttcaccggcggcgacatcctgcgccgcaccagccccggccagatcagcaccctgcgcgtgaacatcaccgcccccctgagccagcgctaccgcgtccgcatccgctacgccagcaccaccaacctgcagttccacaccagcatcgacggccgccccatcaaccagggcaacttcagcgccaccatgagcagcggcagcaacctgcagagcggcagcttccgcaccgtgggcttcaccacccccttcaacttcagcaacggcagcagcgtgttcaccctgagcgcccacgtgttcaacagcggcaacgaggtgtacatcgaccgcatcgagttcgtgcccgccgaggtgaccttcgaggccgagtacgacctggagagggctcagaaggccgtgaacgagctgttcaccagcagcaaccagatcggcctgaagaccgacgtgaccgactaccacatcgatcaggtgtaggagctcactgagcttgtatcctggtgcactctgcgctggaaacttttatgtcgctggcagtcgtatcggttcttgttttaccaatgtttagagttttttgagacctatatgcggttttggttttcagtgcacaattaaaattactgagtaatgtagttgattgggaacagaaatgtttggtgcctggtttaccgaactccagttctcttgtcatttttctttattctatagtctgtattatgtatgcgtatgagtattgagatgattctgcatttgaattgtctgctttgttgctgtcgctgtatgcgtaccaatggtaacagggtagttgtgggaagtagacacggccggttctatgttttcgtgcttccgcggttcaggctggttaagcctatggagcgtacgcacgctcctcccgtctctccgtgtcctgcatgctggcaacaggagtgcggcccagcgcacgccctaatcgacggcggtatatttgtctgtccctccattttgtggtgaggctattcgcaaccgttatccttaaattttttctcctatatcactattcccctatttttccttatattttttcatcttcagcagcggttctcctaaatactccctctatacccactacaactataaatattattttccatatctattcatcatttattaccactttttttcaactaaaaaatactcgcatgcatggattttacggaaggggggctgtcacagtatccccttgatctgctgtgagagaaaagggggacactaggtagggtgcaaggtagggagcagcggtgcgggtggtagcgtggttactgcagccgctacgacgtgagcagtgttaggggagaggatggaagggtggtgcgctgctgcagataacccgagcgccaaacactcatgggtgataattaggtataagaaaagatattttatggttaggagagtatagagagaatttagtggtaacttctatggaagatggaaaaataggggtgaaatcggaccgcgatcgcttaattaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccccgatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggagctcgtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctgcaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgtctcctaatatatggttgattgttcggtttgttgctatgctattgggttctgattgctgctagttcttgctgaatccagaagttctcgtagtatagctcagattcatattatttatttgagtgataagtgatccaggttattactatgttagctaggttttttttacaaggataaattatctgtgatcataattcttatgaaagctttatgtttcctggaggcagtggcatgcaatgcatgacagcaacttgatcacaccagctgaggtagatacggtaacaaggttcttaaatctgttcaccaaatcattggagaacacacatacacattcttgccagtcttggttagagaaatttcatgacaaaatgccaaagctgtcttgactcttcacttttggccatgagtcgtgacttagtttggtttaatggaccggttctcctagcttgttctactcaaaactgttgttgatgcgaataagttgtgatggttgatctctggattttgttttgctctcaatagtggacgagattagatagcccggaaatttaccggtgcccgggcggccagcatggccgtatccgcaatgtgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccggggatctggctcgcggcggacgcacgacgccggggcgagaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgcccatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctagatttaggtcgtgaagatgggctcgagatcgttcgtaatctggcggcaaagtctgatattccaatcataattatcagtggcgaccgccttgaggagacggataaagttgttgcactcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaatta

SEQ ID NO: 27 - DNA, 16054 bp, synthetic construct
  misc_feature 1..16054: note = Recombinant vector
  source 1..16054: mol_type = other DNA
attcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgctgcaggaattggccgcagcggccatttaaatcaattgggcgcgccagctgcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaactcgagacttttcaacaaagggtaatatccggaaacctcctcggattccattgcccagctatctgtcactttattgtgaagatagtggaaaaggaaggtggctcctacaaatgccatcattgcgataaaggaaaggctatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgaacaatcccactatccttcggtaccggacccggatcctgaggtgtggcttgtatgtttcctgaccccctggtggtgttcccagtttgcaatacatcattttctgtagcccttgtacttgctgtgatccctgtttattgttcagggaaaacttagtgcagtgtattagaaatatagaaacctcacatttcgaagagcagaatagtgtttgataacagtttttttaacgcaacaacgtttgttagactgtgtccagcggccagcgggtagtgtaaaataggtgacgcgaaactatatgtgatattgtttgacactatttgcagagtatagtttaaaatagggggtgtgagtaatctgctgaagatagccttaagagttaagataccaaggtatagtttatcatatgcaaaagaaaagaaaaaaaaggaaatacaaaccattgtttttatgttggttagagctaggtaattatctcttgaccagtgtatttccaaacctgttcttctcagtttctgtgctccgtcgatactgaacattgttgttcatttctcatttgttgaacgatggtatttcaggagctagagaagaggagagaggagcttgagtctctgttaactgctgaccggatccgctgcgtttgggggcggcaggatctccgaccggatccgcccgccgtgcccgcgaccggatccgctgcgtttgggggggggcaggatctccgaccggatccgcccgccgtggccgcaaccggatccgctgcgtttggggcggcaggatctccgaccggatccgcccaccgtggcggcgaccggatccgctgcgtttgggggcggcaaggaagcgggtggggcttctagggttcaggtggcgggcgtcggggtagagagtgcctgcggcgattctggcgggcatgcgcagggtggggcaggggtgtggtcgagcgggccatgcacaaccttccatccgcgggattttgcgggcgttgctggcgcgacggatttgcaggagttgctggcgcggagctttgagggcgcggccgatttggggactgcgggcgttggcgattttgcgggcgttgctggcgcggcagattctgcgggcgttgctggcgcggggactgcggacgcgacagctgtgctcgcctgcggccgcgaggaaggcgacggggactgcgggcgcggggagggaaatcgcgggcggagcgcgcgatggcagaacacacagaactatggacgcctacactaagggggtgtttggtttctagggactaatgtttagtcccttcattttattcctttttagtatataaattgctaaatatagaaactaaaataaagttttagtttctatatttagcaattttagaacaaaaatggaataaaatgtagggactaaacattagtccctaaaaaccaaacaccccttaaatacataagaagtagtagagattattattatttgtgtctattgatatgatcattatgatattatattttactgttttaatctaaatatagttattatattcagataattattttattaaatccaaaaatatttaacttctaatgtaataaggagagaatactctaatatcataataatttcttctaaagtgaccctcggatttggagatgactgacagggagggctgtgcacccttctttctttccttttcaattgaagaacttgggttgtgcttgctcacacaaccgaatgaccgatgcacaccgacgcgcacggagcggaccagcacagcccggaaactgccctacgccgacacgttttccggtgcggcccagcagcagcagggaggaaggttcgctcggtcgctgggcctggctccgcgtgccgtccctcctataaatgcgagtcctcgccgagtccactgggcaccagaagctcactcactgctcgttgccggctgcccccgcggccccggccagtccatcccctcgctcgctccccaactccagcaggcagatcagatacatccatccattcgcgcaccggaaggtgagcgccgtgaacgaaccatccgccctgctagctgcgatctgtagccttgcgtcgctttcgcgcctagatcgtcacgtcacctatcacgatccgtgcggttctagatctgtggtttttccttcccctggtggtcgaatccttccatccaccagaccaccacgggacctcgtggattccttttggttttcctgtgccgagagccaaaatcgagggggggggcttgttttttattggctcggtctcccgctgtctcgtgatctgatttgctgtagtaatcagcaggaaaggaagggttgaactaagagcgccgtggcggtttcgtcgtcgctgaacccggacgcgccgctcttcatcccggcggcgctgctgcaggtggaggacttctcgccgcagtggtgggacctcatcaccaccactgcctggttccgcgaccactggtcccgcgagcgcgcccacctggacgagatcgccgagcagatcgacgcggccggcctcctccccgacgacgaggacctcttctacgacgaccaggtcgagcagggccccgtcgccgccgcccttaagataggtactgatgtctctctctctctctctctcttactctcccctcgattttagatctgcctgaaggacgaatcatagtgacctcacgttggtgcgtttttctccaccagattcggtgctcaaggcgctgtaaaccatggacaacaaccccaacatcaacgagtgcatcccctacaactgcctgagcaaccccgaggtggaggtgctgggcggcgagcgcatcgagaccggctacacccccatcgacatcagcctgagcctgacccagttcctgctgagcgagttcgtgcccggcgccggcttcgtgctgggcctggtggacatcatctggggcatcttcggccccagccagtgggacgccttcctggtgcagatcgagcagctgatcaaccagcgcatcgaggagttcgcccgcaaccaggccatcagccgcctggagggcctgagcaacctgtaccaaatctacgccgagagcttccgcgagtgggaggccgaccccaccaaccccgccctgcgcgaggagatgcgcatccagttcaacgacatgaacagcgccctgaccaccgccatccccctgttcgccgtgcagaactaccaggtgcccctgctgagcgtgtacgtgcaggccgccaacctgcacctgagcgtgctgcgcgacgtcagcgtgttcggccagcgctggggcttcgacgccgccaccatcaacagccgctacaacgacctgacccgcctgatcggcaactacaccgaccacgccgtgcgctggtacaacaccggcctggagcgcgtgtggggtccagacagccgcgactggatcaggtacaaccagttccgccgcgagctgaccctgaccgtgctggacatcgtgagcctgttccccaactacgacagccgcacctaccccatccgcaccgtgagccagctgacccgcgagatttacaccaaccccgtgctggagaacttcgacggcagcttccgcggcagcgcccagggcatcgagggcagcatccgcagcccccacctgatggacatcctgaacagcatcaccatctacaccgacgcccaccgcggcgagtactactggagcggccaccagatcatggccagccccgtcggcttcagcggccccgagttcaccttccccctgtacggcactatgggcaacgctgcacctcagcagcgcatcgtggcacagctgggccagggagtgtaccgcaccctgagcagcaccctgtaccgtcgacctttcaacatcggcatcaacaaccagcagctgagcgtgctggacggcaccgagttcgcctacggcaccagcagcaacctgcccagcgccgtgtaccgcaagagcggcaccgtggacagcctggacgagatcccccctcagaacaacaacgtgccacctcgacagggcttcagccaccgtctgagccacgtgagcatgttccgcagtggcttcagcaacagcagcgtgagcatcatccgtgcacctatgttcagctggattcaccgcagtgccgagttcaacaacatcatccccagcagccagatcacccagatccccctgaccaagagcaccaacctgggcagcggcaccagcgtggtgaagggccccggcttcaccggcggcgacatcctgcgccgcaccagccccggccagatcagcaccctgcgcgtgaacatcaccgcccccctgagccagcgctaccgcgtccgcatccgctacgccagcaccaccaacctgcagttccacaccagcatcgacggccgccccatcaaccagggcaacttcagcgccaccatgagcagcggcagcaacctgcagagcggcagcttccgcaccgtgggcttcaccacccccttcaacttcagcaacggcagcagcgtgttcaccctgagcgcccacgtgttcaacagcggcaacgaggtgtacatcgaccgcatcgagttcgtgcccgccgaggtgaccttcgaggccgagtacgacctggagagggctcagaaggccgtgaacgagctgttcaccagcagcaaccagatcggcctgaagaccgacgtgaccgactaccacatcgatcaggtgtaggagctcgttcgctgggggaactcatcaggaaggctgctgcccctcttgcagccttgctcctggctgccgccgctgtcgtggtctgctctttcaagtcgaagtaacggtggttcgagctagtggatagtgtggctcaactgtagaagttccttttgtatagcaagcaagtaaaaaaaaaaaatgaccaaaaaatataacaaaatgcagctgtaagtttactgctgctctctaagtcgtgttcagtcatccagtgtgtctagtctagggaaaccccataaaaatggtgaaggtggaatcccatcccagtgtcataattaaggatgcacttcttctgtaagcaaatgtatgatgtacaatggccggccggcagtctaaatgttacaactagctcttcttggtgaattcaccggtccacactgatgtgctgctatgtatcattactatccagttagggcttgttcggttattcctacgccatatggattggacgggattggaaaattttagtagacattttgacttctatggatttaaacccacccaatcccctccaatccacatggattgagatgaaaccgaacaagcccttagttggatggatggatgatctcacgcgcttgagtttatctagttgctgatatgggagagcccctcaacacctcaaatattgtggataagtttaatcctacactgtcagtcttcagttataaggcacgcactcttcgacgttgggcgctgtcttttgtttcaaaggttgaggcaacctcaacctcgtttaaaacagagagcaaagctaatttccaaactgatgtaagtcatgtgtccttaattaccaaagtagcaatgatgacattgttatgttgtgtgctaatgagccattacgtgaacatcagttcctgccttgccgcttcgttcagccgggccggggtatattttgagtcactaaaaggacacgtcggatgaatggaaaattgcctcttgtctttaccaagagtgtcgcccgcgagcggaccgcgatcgcttaattaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccccgatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggagctcgtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctgcaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgtctcctaatatatggttgattgttcggtttgttgctatgctattgggttctgattgctgctagttcttgctgaatccagaagttctcgtagtatagctcagattcatattatttatttgagtgataagtgatccaggttattactatgttagctaggttttttttacaaggataaattatctgtgatcataattcttatgaaagctttatgtttcctggaggcagtggcatgcaatgcatgacagcaacttgatcacaccagctgaggtagatacggtaacaaggttcttaaatctgttcaccaaatcattggagaacacacatacacattcttgccagtcttggttagagaaatttcatgacaaaatgccaaagctgtcttgactcttcacttttggccatgagtcgtgacttagtttggtttaatggaccggttctcctagcttgttctactcaaaactgttgttgatgcgaataagttgtgatggttgatctctggattttgttttgctctcaatagtggacgagattagatagcccggaaatttaccggtgcccgggcggccagcatggccgtatccgcaatgtgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccggggatctggctcgcggcggacgcacgacgccggggcgagaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgcccatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctagatttaggtcgtgaagatgggctcgagatcgttcgtaatctggcggcaaagtctgatattccaatcataattatcagtggcgaccgccttgaggagacggataaagttgttgcactcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaatta

SEQ ID NO: 28 - DNA, 22075 bp, synthetic construct
  misc_feature 1..22075: note = Recombinant vector
  source 1..22075: mol_type = other DNA
attcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgctgcaggaattggccgcagcggccatttaaatcaattgggcgcgccagctgcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaactcgagacttttcaacaaagggtaatatccggaaacctcctcggattccattgcccagctatctgtcactttattgtgaagatagtggaaaaggaaggtggctcctacaaatgccatcattgcgataaaggaaaggctatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgaacaatcccactatccttcggtaccggaccctatagaatagctcactatcctatttattatagtttaagtatatagccaatattttaaatttactatttattaaattctagggaagatagtctcaattcataactttattataatacgtttgaaattttaaatctttaggaaattttcttaattcacctagatacgattctggagtgttacaagctgcgaatatactggtgccattgagtatacataaatggatttaggtggtgctcaataggtgaaaatgagatactaatcacttaaatttcaaaatttctatggtgccactgtactcggataggtctatctagggctggacaaaatgctcgtggctcgctggctcgctcgtttcgtggtcagctcggctcggctcggatcggctcatttgaattttgtcacgagctgagctgacattctagctcggttcgttaacgagccagctcgcgagctaaacgagctaccatattctagtaaaacgaaattatattcatatcatttatagaataattgatgaacatgttatatatatgtgagatgtctatggcctatgaattaaactaatgattaatgaactatgcctatgtgttaatttggtctatgcaaatataattatgggttaaactgatgaacatgcatgtgaattgtgaattaatgagtgatgaattgtgctaatttggtgttatattgacatggtttgtgaaactatgagtataattactattttctattgttaaattagtttgaaattaactaaaaaataattattatatacattttattttttttctgctctggctcgcgagctaaacgagccagctcgacctcgtaaacgagccgagccgagctgactctgtggctcgttaccttaacgagccgagccgagctggctcgttagcttaacgagccagctcgaactcggacgagccgagccgagctggctcgttatccacccctaggtctatctagcttctgatgtttgcaaaccttagagttggagtgttcagccagctactcctttgctttgctgaataaccataccaaacacgcccatattaatacccgctcggcggtggttctgcaatcaaacgcaggccgcagtcgcgtgcggaactagaggtccttcagagaagtgccgtgccagtgccaccgccggccgcatcatcgttccgcccccctggtacgagcacttcgcagagctgcaacctacatcccttttacataaatctattgtctcgtattgccgttgacgccggaatagtcttcgcatcccttttacataaatccgatgttttctttctccgattcctttgaggaatcatcacgggtcagggcaggtgttctgccgtttgcccttttctttatattctccttagaagaaatatttagttggaggctggacatagccggaggagctaactaatcgagcggtgtactggcaaaacaaaaggagcggagcaagaaaggggagaaaaaactagccactgccggagcgctattggccgtgttgggcctggaagcttgcatcaatacttccctcgccccgatttggttccaaaatcatacaagtcccaaagttgtcaagatattggaggtatgcaagcgacttggatctcaaaatagaagaaatttcggatctgagcacaaatctgagttgaaaaaactgcaactcaaaatcatcaaaaaaagaagaagaaagaaacgaatatattcgctcctcttctcagccgaacccaaaggaattgaatccaaaccctgggtaggcagacagtgagatatggaggagagcaggaggcgaacaagagaggctgcggccacgaatatctcacgaacaagcacatcatgggtccacggagcgggcagggtgacgggctcccgacggcgagctacatctcggaagagcaccagggcagcatgtcgtgttgggcaggttggccgtctggcggacggcggacggtgactcgtggtcagggtgcacctgctcgattaaggcgcctgactactcatgtcttggtctctttgcttgtgtttgctatatgctgctcgtacctcatgagcatactaagttgactgctcagtctgctgagtctgtttttctagggtatagtgctgagcacaagggatatcattgttgggatatgattgctcgttggatgagggtctcttgggatgttgtctttgatgaggctcattctttttattcttgtccttctttcgatgctttgtcaacatccttggttgatcccatctcttttctatattttctagatgcccgtgttactattggacctgcctcacgcttggtgcgcccacgatagtagccttagctccttctgacatgttcatctctctttcggtgccttcctttgtggtgccttctatagtgttttctttggagcctgctgctttagcccctgactacgctatgaacacttgtctacacccgccgggtcatcaattcttttggtacaccatcatcctctcatgcgttgccctcttatgatgtgcgctcttctgcaactcattcattttcttgcgatttacctttgactgatgctccctattcatctctggatccagcttcctcagttgactctttgctggagccacctcttagacggagtcatcgttttcgtcagccacctaatgggtactctccttcaggtttagtcgctaccgttctttctgagctgacttcttatcatgatgctattcttcatctgtaacgacaacatgcgatttctgaggagattgctactcttgagcgcactagcacgttggaacttgttccttgtccatcacgtgtttgtcctatcaccagtatgtgggtctataaggtcaagacccgttctgatggttctcttgatcgctataaatctcgtctagttgcccaaggcttccagtaggaacatggttgtggctatgatgagatttttgcacctgttgctcatatgaccactgttcgcactcttcttgctatggcctctgttcgtgcgtggtccatctctcatcttgatgtcaagaatacctttcttgatggtaagctacttgagttctatatgtagccatcgcctaggtattctatttctgcttgtatggtttgttgtcttcgccgttccccttatggcctcaagcaggctccacattcttggtttcagctctttgcttctatgataactgttgttggtttttctaccagtaatcatggtcctgcactctttgtgtactacctcctctcggggtcggactcttctttatgttgatgatataattatcactggagataaccttgagtatgttgactttgttaaggcacgtcttagttatcattttctcatgtctgatcttggtcctctgtgttactttcttgggacaaaggtttcttctttgtctcagggcctttatctatctcaagaggagtacattcaagattttcttcatcgggcttctcttaccgatcactagattgttgagactcccaagcagctcaatcttcaccttagtgccgatgatggcgagtcttttcccgaccatactcgttatcgtcaacatactgtaggaagttttgtttatctctgtgtcactcgtcttgacatttcatatgttgtgtgtatcctgagttagtttgcttcagatcccatccaggtacactatagtcacttgctttgtgtcctacaatatctttgtggaaccatatctagatgtatgttctttccacattctagctcgttgcaactgcaatcttgttctgatgctacttgggctagtgattttttcgatagttggtctctttctcaatattgtgtttttcttggtggttctctcattgctcggaagactaagtagcaggtagcagtttctcgtttgagtaccgaggctgagttgcgtgctatggcccttgtgactgcagaggttacttggttacgatagttgcttgaggattttcatgtttctgtttccatgacgactccttttgtctgacagtacaggtgttatcagtattgctcgtgatgcggtgaagcatgaggtcaccaagcatattggagttgatgtttcgtatacacgagctgaagtctaggatgatgttatcttgatttggtatgtgcctttagagcttcagttggctaatttcttcacgagggcacaggctcgcgctgagcataaatttttcctctcaaaactcagtgttatagatccaccttgagtttgagggagtattagatagatatgggtttatttgtatttttccattttataagggtattagatagataggcaacgactgctatgcaagtagtcattctgtgcaagcgtgcaagcaaaccatctgatccattatatcgtgatccaaccgtgggtcacatttaacacttaaacccttccaccaccaactcaataatctttataaaaaaacccctaacaaacaatggttatatctgtggttggatcgtaatctaatagatcagatggtttgcttgtacgcttgcacagaatgactgcttgcatagcagttgttgcctagatagatatgggtttatttgtatttttctcttaagggtttttgtgtatatttgtactcatgtacctatatatttgtgctagttgaccccataatgaatagacctgctattcataatatttgcaaaccatgaaaatttgattattacgaactatccaaatactcgaacacatgggcattatagctcacaaaaatggaaggttgagctgctgcttgaagaacctcaacatctttgaacaacaacctcaacgaaacttgtatatgaaccaacttccaaacaatcccttgtggaaggatagtaatgacttcagggcattgatcacacatatccgacggtggaactactgtaacaaccctcttttctgtggaatatagttgaaactctacaacttgaccaaaaccaagatgacgacatatggtggaactaacaaaacaagaggactacactacctcattagcttattaagcacaatctcttggcaccacaacaacgaacaacaaaaccatcatttggatgctctgtgggcgactaaatgcaaattctttgcatggttgatcatcccaaattggtggcacttagctataggctagcagtgagaggatggccgaacaacatgcattgtccactatgttggtgtagccatgagaccaaccaccacataaatgccaaacgttcattcaccaaaaaaatctaggcaacaatggcttggatttcttacctgcagctccaccaagctaactggagttcaattaggtcaacgtatgggtggtggtcgagtatagcagtcacaaatgatgttctaaagatggggttgtgttaacacatcttgcttgtagcacgagaacactggaaggagtgaaaccaaagaatctttcaacacaaggacctatcaacgctatccatgattgggaaattcaaggacgaaactagaatttgggtgaacacatgcacaaggcacctaggagagcctttcttttgtactgttaatccctttttaaactctctctgtccttaggagttcgtttcttccgctctattcaatgaagttaggcacaatcttgtgtgatttcattagaaaaacacaagtaaattgcatggtcagtacttgaagtattacaggaatctcgtctgcccccaaactattaaaccttatatttggctccctaatgtacttaactgatctcattctggtcaaactaaacatggtgatggcaaggagccgatatggtcgcccatgtggatgtgatttaagcaaaaaatctcatggtccatagctgtgtcaacaagccaacatgccatcgcttccttatgccgagactgcccatgtcgctcgcttttactgtcatcatcatcaaactgcctgtcatgtctacggatgccatgaccgctgtcacacatgatgtggagatgaacctgtccatcaacttccacgtgctgccactatcgctagctgacaccgtcttggtcattgctgtgtagggctaggctaagagtcgctgaatgatcctttcgctctcctttacaggaacatgctgtttactttgtgtcgccaaggcgtgctagagtacctcttctacacctccagcaccagtagccttattgttagcttgcacatcccacataagcaggccgatgtgaatgataacttcagggacgtcgacggcatgtcactgccaagagtcatttggtgggaagcgttgtcatgccatctgtcgtgccattttgtcctcagttcgaccgccattaccgtgagcacaacctttgcgcatggttggccgcttccatcacccttattccgtttcctcgtgttggtcttgccccaaggctatggttagcagaccgtgcatatggccggcaaaagactattttgcactgtagattgcactctttatatagtgaagtttaaaataggagatgagatgaataaggctgctggagatagcctaaacccttgcagctcgtgcttgcatcgggggagccaaaaggcgtccacctccaccatcgccgaagcactgagcactactctggcttgtgtttcagcaccacaccgcagagtgcttagggccaccaacctcctcttgcctctgtgcccagagcaccatcagctctgctgcctccctctgttccttgtgcttgctaggcaggcaattccgagctggggcccaacttgtaacgctgatttcaccatcttgccactgccgggcaccaagtggacacatttgacttggcctagtgggttttctgcataaatcacatacatgtggatgccatatcaggctctttggtgttgtcgtgtctactttcgacaaggatgagatcacttaaacatattagggagccaagtatgtaatttcatagtttagggacctacacaaaaatcgtataatactttagaacagccgtgcagtttactcaatcaacacatacaaagtcagatcttaagctctgatacttcaaaggaatggttgagcccagttgacaaacaatcttgcttcattcattgaattgtttataggagtggctatgtaactactgggtggttttgtttgacctgtcatccaaattgtgtagtcaaccataaacatacacgtcacacaatacattttggatgtgacagataggatttaggcgagagaatgtacaatgtcactgaaaaattaccactgtatggaaaggacaatctaagtgaaaagagaaccagggcctaatggtttcaggacttcaaactccggccaaatgaatttacagtgcttaaattaactcatgttaatcatgatagccaaagcatgggcaaaagagaaactatgaataaatcgacaatgtattctatatagcagtaatataccatgtcacgagcttttacactaatgggctgtatttttctgcagttattttaactggcaatattctatgtcacagtaatatttgttaaattttttccagaatagcaactgaactagaagtctagtatttcttaattggataacaaaaggaattagtgtgcatttggcttacgaacaatcagtcacccaacattgaatttgaagttctgtttcctctttgttcagacgacactctccaaatgaatgccttatattttgtgttgctcctcttttctgcagagtgttcagtaacttcttccgatgtaaaccatggacaacaaccccaacatcaacgagtgcatcccctacaactgcctgagcaaccccgaggtggaggtgctgggcggcgagcgcatcgagaccggctacacccccatcgacatcagcctgagcctgacccagttcctgctgagcgagttcgtgcccggcgccggcttcgtgctgggcctggtggacatcatctggggcatcttcggccccagccagtgggacgccttcctggtgcagatcgagcagctgatcaaccagcgcatcgaggagttcgcccgcaaccaggccatcagccgcctggagggcctgagcaacctgtaccaaatctacgccgagagcttccgcgagtgggaggccgaccccaccaaccccgccctgcgcgaggagatgcgcatccagttcaacgacatgaacagcgccctgaccaccgccatccccctgttcgccgtgcagaactaccaggtgcccctgctgagcgtgtacgtgcaggccgccaacctgcacctgagcgtgctgcgcgacgtcagcgtgttcggccagcgctggggcttcgacgccgccaccatcaacagccgctacaacgacctgacccgcctgatcggcaactacaccgaccacgccgtgcgctggtacaacaccggcctggagcgcgtgtggggtccagacagccgcgactggatcaggtacaaccagttccgccgcgagctgaccctgaccgtgctggacatcgtgagcctgttccccaactacgacagccgcacctaccccatccgcaccgtgagccagctgacccgcgagatttacaccaaccccgtgctggagaacttcgacggcagcttccgcggcagcgcccagggcatcgagggcagcatccgcagcccccacctgatggacatcctgaacagcatcaccatctacaccgacgcccaccgcggcgagtactactggagcggccaccagatcatggccagccccgtcggcttcagcggccccgagttcaccttccccctgtacggcactatgggcaacgctgcacctcagcagcgcatcgtggcacagctgggccagggagtgtaccgcaccctgagcagcaccctgtaccgtcgacctttcaacatcggcatcaacaaccagcagctgagcgtgctggacggcaccgagttcgcctacggcaccagcagcaacctgcccagcgccgtgtaccgcaagagcggcaccgtggacagcctggacgagatcccccctcagaacaacaacgtgccacctcgacagggcttcagccaccgtctgagccacgtgagcatgttccgcagtggcttcagcaacagcagcgtgagcatcatccgtgcacctatgttcagctggattcaccgcagtgccgagttcaacaacatcatccccagcagccagatcacccagatccccctgaccaagagcaccaacctgggcagcggcaccagcgtggtgaagggccccggcttcaccggcggcgacatcctgcgccgcaccagccccggccagatcagcaccctgcgcgtgaacatcaccgcccccctgagccagcgctaccgcgtccgcatccgctacgccagcaccaccaacctgcagttccacaccagcatcgacggccgccccatcaaccagggcaacttcagcgccaccatgagcagcggcagcaacctgcagagcggcagcttccgcaccgtgggcttcaccacccccttcaacttcagcaacggcagcagcgtgttcaccctgagcgcccacgtgttcaacagcggcaacgaggtgtacatcgaccgcatcgagttcgtgcccgccgaggtgaccttcgaggccgagtacgacctggagagggctcagaaggccgtgaacgagctgttcaccagcagcaaccagatcggcctgaagaccgacgtgaccgactaccacatcgatcaggtgtaggagctcgacgtacaaatctcatctgtgccttgctctagtttcccaaatggaattaactatgcatgatttgtttggaaactcttattgcatccatccagataatgcatccaccataaggtaatatcttgatgacatctgtgcctgatggtgtaccaaatgtctctatctctgcattgagccacgagtaggaggatagcctaggggtgccttgactccaaagttgtattgaaaaagatggatgaagcaggcaaatgctgcctgaatccatgactcagggcacagattttccactcaaaggaagataagattgcattacttcatgatcttttgaactgcctctgcaagacgggactcggatagtggatgcaaagatctaatactggcctcaggcaacgagttgtttcactcgaaagtctagaaatgaccgggctcaaattttgcaccccaaggaaagtgagtttgcattacttcatgaccttttgaactgcctctgcaagactggactcagattacgcttgattggttgccggcctcaccttcgcctggcttgcgcgagcctgcgtctatagaaatgcgccggactcacgtctccgtcgatgcaggcattcgactgaaaaaacatttaaactgcacccatgcgtgcgggctgagcttatgtcatacaagtaaccaatcacaggcttaagttcagtcaacgcatgcgctaagcttggatgtggctgaccgggcaaccaatcacacagatagtggatgcacggatctaatattggctaatttggttaaacttgtctaaccttagacgtggcaagtgagtcagcggatcaaatctgctctaaaattgtctgcctcctagatgtccttggtgttccaagatttaatcatcactgcactatttctttgcgttgcttcgctgcagcttcgcgttacttgcattcgcttaatcaggattactttgatcaactaggtttctaacttctactaccttcacttgcacagggtgcccgtcctgctagccggtgtgcttgctgtgcgatcgtttggcatgtgcttgttgaggggttgctaggggattggagaggattgaagggattaaatctcctcctattcaattttgaataggaggggatttaatccccttcaatccccctcaaaccactagtaaccgaacgtggcctgagggggcgggcgagtctttatattgaatgaaactacataaaatagcatgccgtctctgtcactggcaatggacggtggtgcctagcgcaactcagcgcacaactgtgtgtcttgatttttcttctgtttatcacggcattagtgccatgccgttttatgttacagtgttgtgtgctcgcaagcatccgaaaatatgcgtctgagtttagggttgggtcaaacttgtcgaatttggggttctgttataatatgttgagcatgaataaagatggatgctggtgactctgtcgccatcgccgtccatcatgagtgtcctgtaattcaacttatatctatcatgtatgtatgtatgtatgtatgtatgtatgtatatgctgtctactatgcttctttgttttaactgaaatgtgtgttacagtgttacttctctggggtccatttaaaacggcatttcgtttacgataggaaccagccattataatctttaaccaataatttcgctaaccaatttcaactattgcaatgcgaacttaatattatcagatttataaccgaatgcgctatcaaataatcataaggttgtaatcataataatataatataaaataaatgagtgctcgaagtgaaattttagagagcgttataagaaaaattgatgtgatctccaagaataatagcccctcccggctcccggtacaaacatagggcttctttagaatgcaggattgtgagaacataggaataggaaaaatataggaattctataggaatgtatatggaaaacagaggattgaaaaacacagaaaaaatgtgaaagcaagtctttggatgaagcgtaggaaacttataggaataggaattcataacggaccgcgatcgcttaattaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccccgatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggagctcgtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctgcaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgtctcctaatatatggttgattgttcggtttgttgctatgctattgggttctgattgctgctagttcttgctgaatccagaagttctcgtagtatagctcagattcatattatttatttgagtgataagtgatccaggttattactatgttagctaggttttttttacaaggataaattatctgtgatcataattcttatgaaagctttatgtttcctggaggcagtggcatgcaatgcatgacagcaacttgatcacaccagctgaggtagatacggtaacaaggttcttaaatctgttcaccaaatcattggagaacacacatacacattcttgccagtcttggttagagaaatttcatgacaaaatgccaaagctgtcttgactcttcacttttggccatgagtcgtgacttagtttggtttaatggaccggttctcctagcttgttctactcaaaactgttgttgatgcgaataagttgtgatggttgatctctggattttgttttgctctcaatagtggacgagattagatagcccggaaatttaccggtgcccgggcggccagcatggccgtatccgcaatgtgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccggggatctggctcgcggcggacgcacgacgccggggcgagaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgcccatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctagatttaggtcgtgaagatgggctcgagatcgttcgtaatctggcggcaaagtctgatattccaatcataattatcagtggcgaccgccttgaggagacggataaagttgttgcactcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaatta

SEQ ID NO: 29 - DNA, 7910 bp, synthetic construct
  misc_feature 1..7910: note = Cry1Ai expression cassette
  source 1..7910: mol_type = other DNA
agctgcttgtggggaccagacaaaaaaggaatggtgcagaattgttaggcgcacctaccaaaagcaactttgcctttattgcaaagataaagcagattcctctagtacaagtggggaacaaaataacgtggaaaagagctgtcctgacagcccactcactattgcgtttgacgaacgcagtgacgaccacaaaactcgagacttttcaacaaagggtattatccggaaacctcctcggattccattgcccagctatctgtcactttattgtgaagatagtggaaaaggaaggtggctcctacaaatgccatcattgcgataaaggaaaggctatcgttgaagatgcctctgccgacagtggtcccaaagatggacccccacccacgaggagcatcgtggaaaaagaagacgttccaaccacgtcttcaaagcaagtggattgatgtgatatctccactgacgtaagggttgacgaacaatcccactatccttctgcctaattagctaacggaccctctgcctttctgttcttcaaacgatgtcacatgtctgcgctggacaactttcttgttgccgcctgtcgcttgcgctgtgctgactggacgcagctccggaggtttggttgtgcttggttttcgtagagaactcgccacttgccgcccgcacgttcttggtgtttcctcctccccgctgtgttctgcgcacgggctttttctgagagacccatgtttcccttttacttttataaacagtatacatgctatgtttctagaaggaggggaaacctaatccccctaatccaatggcggggaggaaatagggtggggtggggtggggggagggaaatatctcgctactttttaatccggacaagctcatttgcgtttgcgtctgaatgatgatgactgcaatgctgatcgcacctcgggtgtcggatcaccagcttttggctgctctcaccaaatcagctgcaagaagattagagcacaaaagaattacagaaagagagcctttttcttttcttccttgtggggttcctttcatttcgtgctctcctttctctgccagccagtccgtccttgcgtccactgcacctgcacacaggtcaccccgacccgcactgttctagactccattagaaaaaaaaaggtctgaacctttccgaaaccagccagccattggtctggcaggccagcatatgctaattggatttttttgccgcatcattgagtgcgccatcaggatttggaaatcctggttttgagtaatacagtaatttggcattatccattgccgaattcccaagctccgtcagcttgaacgtggacccctaccatctgcaccagctcggcacctcacgctcgcagcgctaggagcctaggagcagctgcccgtctatttattggtccctctcccgtcccagagaaaccctccctccctcctccattggactgcttgctccctgttgaccattggggtatgcttgctgccttgctctcctgttcatctccgtgctaaacctctgtcctctgggtgggtttttgctgggattttgagctaatctgctggtcccggtagaaaaagatcatgtcccctgacgtgctcaagcgctcgccttagccgcgtccttgccccccgccattttttgcggtttcggtgtgttcccgtgactcgccgggtgcgtcatcgcctgaatcttgtctgggctctgctgacatgttcttggctagttgggtttatagattcctctgatctaaaccgtgcctgtgctgcgcacagaactctcccctgtcctttcctggggttttggttacgtggtggtagtaagcttggatttgcacatggataaagttgttctaagctccgtgggttgcttgagatcttgctgttattgcgtgccgtgctcactttttttgcaatccgaggaatgaatttgtcgtttactcgttttggtggattattagcgcgaaaaaaaaactcttttttttttgttcttttactacgaaaagcatcttcttggattttgctatcttcttttactacgaaaaactcttgagtctaggaatttgaatttgtgatgtccattcttgcagtgcgctgtgctttattgggaagccaaatcctattattttctgcctctagggtctgaatggaatcagtactcttgagacagaaaatcaatccaatcaagttgatttctttctttaaaaatattatcacagaactaagtgcttgtgcggaatcagtactggcttttgtttggtggaggatcaatacttgcttttgtttgggggtggcaactgttttgctataagattccatgtgttcctgttgagatgaatcatatatagtatagctgcatactacaaatctgtttttcaaatttaggttgctttggcatgatctatttttttgtcagacagactttctaagtggtagctcttgatttcttgttcttgtacaactggtgctgctgaatcttgaccgtatagctcgaattgcagtattctgaaccatcgagccaaggctgccaagctgactcgcctccacagtcttcgcgaacgccttggtgccaccttctcctcccatcccaatgaactgatagcactcttttccaggtgggcttaccaaaatcatataacttgcatttcattcggtactgaaagttgttaatttgttattctcttcatgcctgtcttaatagcacacccagatgtaaacacgagattatgcaacttcttacttggtttcttttgttggcaccatcatgcatgctaattgctaaggatgttacctattcatccttgactcatattatcatatgtaatgattttatgatcacgagactattgattgtgaagcatagtatagctgttcttcagtttttgtacccttttgtttttttccttaagctagaactggtacaatttagttgataagacagtgtagtttgtagtacgtcatttgacagattgtttgtctttagctggtaaagtgccatttaatatctgtatccttcagatctaataaaaaggatatgagatgtccatcacaagaggggaaaaattacatgatctgagatgtaacatccgtttttatttgtgaaataccacttctacaggtatcttcactagggtaaaccatggacaataatcctaatattaacgagtgcattccatacaactgcctgagcaatcctgaggtggaggttctcggcggggagaggattgagacgggctacacgccgatcgacatctccctgagcctcacccagttcctcctgtccgagttcgtgccgggcgccggcttcgtgctgggcctcgtcgacatcatctggggcatcttcggccccagccagtgggacgccttcctggtccagatcgagcagctcatcaaccagaggatcgaggagttcgcgcgcaaccaggccatctccaggctggagggcctcagcaacctgtaccagatctacgccgagtccttccgcgagtgggaggcggacccgaccaacccggctctgagggaggagatgcgcatccagttcaacgacatgaactcggccctgaccaccgctatccccctcttcgccgtgcagaactaccaggtgccgctcctgtcggtctacgtgcaggctgccaacctgcacctctcggtgctgagggacgtgagcgtcttcggccagcgctggggcttcgacgcggccacgatcaactcccgctacaacgacctcacgaggctgatcggcaactacaccgactacgccgtccgctggtacaacaccggcctcgagagggtgtggggcccggacagcagggactgggtcaggtacaaccagttccgcagggagctgaccctcacggtgctggacatcgtcgccctcttctccaactacgactcgaggaggtaccccatcaggaccgtgtcccagctgacgagggagatctacaccaaccccgtcctcgagaacttcgacggcagcttccgcggcatggcccagaggatcgagcagaacatccgccagccgcacctgatggacatcctcaactccatcaccatctacacggacgtgcacaggggcttcaactactggtcgggccaccagatcacggcttccccagtgggcttcagcggcccggagttcgccttcccactgttcggcaacgcgggcaacgcggccccgccagtgctcgtctccctcaccggcctcggcatcttccgcaccctgtccagcccgctctacaggaggatcatcctcggcagcggccccaataatcaggagctgttcgtgctcgacggcacggagttctccttcgcgagcctgaccacgaacctcccgtccacgatctacaggcagaggggcacggtggactccctggacgtcatcccgccccaggacaactcggtcccgccccgcgccggcttctcccacaggctgagccacgtgaccatgctcagccaggcggccggcgccgtctacaccctcagggccccgaccttctcctggcagcacaggagcgcggagttcaacaacatcatcgcctccgacagcatcacgcagatcccggcggtgaagggcaacttcctcttcaacggctccgtcatcagcggccccggcttcaccggcggcgacctggtgcgcctcaactccagcggcaacaacatccagaacaggggctacatcgaggtcccgatccacttcccgtccaccagcaccaggtacagggtgcgcgtcaggtacgcttcggtgaccccgatccacctgaacgtcaactggggcaactccagcatcttctccaacacggtgccggctacggccaccagcctggacaacctccagtccagcgacttcggctacttcgagtccgccaacgcgttcacgtccagcctcggcaacatcgtgggcgtccgcaacttctcggggacggctggcgtgatcatcgacaggttcgagttcatccccgtcacggccaccctggaggctgagtacaacctcgagcgcgcccagaaggccgtgaacgccctgttcacctccacgaaccagctgggcctcaagaccaacgtcacggactaccacatcgaccaggtgtccaacctggtcacctgcctcagcgacgagttctgcctggacgagaagagggagctgagcgagaaggtgaagcacgccaagcgcctctccgacgagaggaacctcctgcaggacagcaacttcaaggacattaataggcagcccgagaggggctggggcggctccacgggcatcaccatccagggcggcgacgacgtcttcaaggagaactacgtcaccctgagcggcacgttcgacgagtgctaccccacgtacctctaccagaagatcgacgagtccaagctgaaggcgttcacccgctaccagctcaggggctacatcgaggacagccaggacctggaggtgtacctcatccgctacaacgccaagcacgagacgctgaacgtgcccggcacgggctccctgtggcccctcgcggtcaagagccccatcggcaggtgcggcgagcccaacaggtgcgcccccaggatcgagtggaagccggacgtggactgcagctgcagggacggcgagaagtgcgctcaccactcccaccacttcagcctcgacatcgacgtcggctgcacggacctgaacgaggacctcggcgtgtgggtcatcttcaagattaaaacccaggacggccacgcgaagatcggcaacctggagttcctcgaggagaagctgctgctgggcgaggctctggccagggtgaagaaggccgagaagaagtggcgcgacaagagggagaagctcgagtgggagacgaacatcgtctacaaggaggcgaaggagtccgtggacgccctgttcgtcgacagccagtacaaccgcctccagaccgacacgaacatcgcgatgatccacgccgcggacaagagggtgcaccgcatcagggaggcctacctgcccgagctgagcgtgatcccgggcgtcaacgctgcgatcttcgaggagctggagggcctcatcttcaccgcgttctccctgtacgacgcccgcaacgtgatcaagaacggcgacttcaactacggcctcagctgctggaacgtcaagggccacgtggacgtcgaggagcagaacaaccacaggtcggtgctggtcatccccgagtgggaggccgaggtgagccaggaggtgcgcgtctgcccgggcaggggctacatcctccgcgtcaccgcctacaaggagggctacggcgagggctgcgtcacgatccacgagatcgaggacaacaccgacgagctgaagttctccaactgcgtggaggaggaggtctacccgaataataccgtgacgtgcaacgactacaccgccacgcaggaggagtacgagggcacctacacctcgaggaacaggggctacgacggcgcctacgagtccaacagctcggtccccgcggactacgctagcgcgtacgaggagaaggcctacaccgacggcaggagggacaacccgtgcgagtccaaccgcggctaccgggactacacgccgctgccggcgggctacgtgaccaaggagctggagtacttcccggagacggacaaggtctggatcgagctgggggagaccgaggggactttcctggttgattcagtggagctgctgctgatggaggagtgagagctctacggggaaagaaggagaagaagaagaagaagcccaggccggagaaccatcgcctgcatttcgatctgtttcaccgcaattcgcattgttagtcgtgtattggagttatgtgtacttggtttccaagaactttggttccttctcgtttttttttccttgcttgtttgagcgtttttgggcagcgctggcctggttcctagtatggtgggaattggctgcaccttttgcttcgaataaaaatgcctgctcgttcacctgtcttccagagtgcaatgcgatgttctgttgcccaggtcgtgtggttctgactgatggcgatgttgtgttcttctgttaatcgcctgttttaacgtggtaggctgttgcttgttcttgttgagaaagcttgctgtgccagacatggctgcttgaatacaagtgaaggaaaaaaaaagccatgccaagtaaagttgcacaaaatttcaactgctcagtggaccactggaccatgttcttggttattgcagttgcagggcttcacatggcgtttggacagcagtcttggattgatccataaagaggtggtggttaatgaggacgcaaggccgttccctcagagtcagtcacaaggttgcagaggtcacggttctcttccctttccgcttcctgtcacatcggaattgttgtttacgccatctgcccatcacccaccaagtctatgtttctgtactggatctttcaatggcggaacgcgcttagttcttcgtcacagtcgaatcacatgatctaatcgatgtctttaatctcgctgtaaaaagggtgggacggtgggtgcagggtagggaccagggaaggcctgcctaaacgtatccataaacatgcacagcaaccctaagatattatactgcctacttcctaagatatagttatttctagtctattttttttccgtccacatccaaataagtgataatatatagacatacatatatatactatattcatcatagattaatgaacgaatgtatacttagtttaaacctaattatattttaggaaggatggagtatgaaacatgacaatacaacaaaaaaaaatcatgtaattgcatatcgtcaaagttatctgaagtaaccaatccagggggaaatcccgttagcaaacatacaagagcaccgccccactacatcccagaaaataaaacaaaaccagaactcagatggataaataatactac